SEQUENCE LISTING (107 sequences)

SEQ ID NO: 1 - protein, 527 aa, synthetic construct
  REGION 1..527: note = human gut metagenome
MEKNRAVLYLRLSKEDVDKVNKGDDSSSIKSQRLLLTDFALERGFKIVGVYSDDDESGLYDDRPDFERMMTDAKLDEFDIIIAKTQSRFSRNMEHIEKYLHHDLPNLGIRFIGAVDGVDTESDENKKSRQINGLVNEWYCEDLSKNIRSAFKAKMKDGQFLGSSCPYGYKKDPQNHNHLVVDDYAAKVVQKIFNLYLEGYGKAKIGSILSSEGILIPTLYKKDILKQNYHNSKALDTTQNWSYQTIHTILNNEVYLGHLIQNKVNTMSYKDKNKRILPKEKWIIVRNTHEPIITEEMFQDVQKLQKNRTRSVENIEPNGLFSGLIFCADCKHAMSRKYARRGEKGFVGYVCKTYKTQGKNFCESHSIDYDELEEAVLFSIKNEARSILQQEEIDELRKVQAYDETKSYYEMQLENIKSRMEKIEKYKKKTYDNYMDDLISRDDYKKYVTEYDKEIGGLKQQQELINSKTDLEKEISTQYDEWVEAFINYVDIDKLTREIVIELIEKIEVNKDGSINIYYKFKNPYIS

SEQ ID NO: 2 - protein, 510 aa, synthetic construct
  REGION 1..510: note = human gut metagenome
MNTVIYARYSAGPRQTDQSIDGQLRVCTEFCKQRGLTVVDTYCDRHISGRTDERPEFQRLIADAKAHKFEAVVVYKTDRFARNKYDSAIYKRELRRNGIQIFYAAEAIPEGPEGIILESLMEGLAEYYSAELAQKIKRGLNESALKCQSLGSGRPLGYTVDEQKHFQIDPESSQAVKTIFEMYIKGESNAAICDYLNARGLRTSQGNLFNKNSINRIIKNRKYIGEYRYNDIVVEGGMPAIISKETFCMAQAEMERRRTHRAPVSPKAEYLLAGKLFCGHCKGPMQGVSGTGKSGNKWYYYYCANTRGKERTCDKKQVSRDRLEKAVVDFTVRYILQENVLEELSKKVYAAQERQNNTASEIAFYEKKLAENKKAIANILRAIESGAMTQALPARLQELENEQTVIQGELSYLKGARLAFTEDQILFALLQHLDPRPGESERDYHRRIITDFVSEVYLYDDRMLIYFNISSADGKLKHADLSAIESGVFDAGLISSSSRASSFSTRCALI

SEQ ID NO: 3 - protein, 482 aa, synthetic construct
  REGION 1..482: note = human gut metagenome
MNEKNLEIGAAYIRVSTDDQTELSPDAQLRVILEAAKKDGIIIPQEFVFMEDRGRSGRRADNRPEFQRMISTARQNPSPFRYLYLWKFSRFARNQEESAFYKGILRKKCGVTIKSVSEPIMEGMFGRLVEMIIEWSDEFYSVNLSGEVLRGMTQKALEHGYQLTPCLGYDAVGHGRPYVINEEQYQIVEFIHRSFFDGKDMTWIAREANRRGYHTRRGNPFDTRAVRIILTNSFYVGLVKWNDVTFQGTHECRESVTSVFSANQERLNRIHRPRGRRQASSCKHWLSGLLKCSICGASLGYNQTKDLTKRGHAFQCWKYTKGIHPGSCSVSSLKAEAAVLESLQMILETGEVEYTYEQREKHLDDNKLTLIQKSLERLDTKELRIREAYESGIDTLDEFKTNKARLQRERDQLMEELEELHSQEEPEDVPGKEILIERIQNVYDLLQSPDVDNDDKGNAVRSIIKKIVYIKESKTFCFYYYV

SEQ ID NO: 4 - protein, 406 aa, synthetic construct
  REGION 1..406: note = human gut metagenome
MERTIKVIQPGTVKIPTKKRVAAYARVSSGKDAMLHSLSAQVSYYSNMIQQKNEWSYVGIYADEAITGTKDRRVEFNRLIQDCTDGKIDMIITKSISRFARNTLTMLEVVRKLKNINVDVYFEKENIHSISGDGELMLTILASFAQEESRSVSENCKWRIRKGFEQGELINLRFLYGYRINKGKIEIYEKEAEIVRMIFDDYLNGEGCTRIGNKLRKMKVNKLRGGMWNSERVVDIIKNEKYTGNALLQKKYVKDHLSKKLVRNKGILTQYYAEGTHPAIIDIKTFEIAQKIMEANRTKFQGKCGSNRYLFTSKIECGICGKNYRHKDREGKSTWVCANHLKYGNSRCIAKPLNEEKLKKLINEALELKYFDEEIFIRNIKRIKVTGNQTIEFILKDGKVIEEGMI

SEQ ID NO: 5 - protein, 401 aa, synthetic construct
  REGION 1..401: note = human gut metagenome
MKKIKIDRAIQERPATRKQTRNEKIRQSLTEHVDVQVIPAITDREGYEKPKLRVCAYCRVSTDMDTQALSYELQVQNYTDYIRGNDEWRFAGIYADRGISGTSLKHRDEFNRMIEDCKAGKIDLIITKAVTRFARNVLDCISTIRMLKQLEHPVAVYFETERINTLDTTSETYLGLISLFAQGESESKSESLKWSYIRRWKRGTGIYPAWSLLGYEMGEDGKWQIVEAEAELVRIIYDMYLNGYSSPQIAEILTRSGVPTATNQTVWSSGGVLGILRNEKYCGNVLCQKTMTVDVFSHKAIKNTGQKTQYFIEGHHDPIILRSDWDRVQQMIDEKYYRKRRGRRTKPRIVLKGCLAGFTQIDLDWDEDDIARIFYSTTPAAEVATPAMADHIEIIKVKGEN

SEQ ID NO: 6 - protein, 476 aa, synthetic construct
  REGION 1..476: note = human gut metagenome
MKTAAAYIRVSTDDQVEYSPDSQIKLIRDYAKRNDYILPDEFIFRDDGISGKSAKHRPEFTKMIALAKSPEHPFDAILVWKFSRFARNQEESIVFKNILRKIGVEVRSVSEPISEDPFGSLVERIIEWTDEYYIINLSGEVKRGMLEKISRGQPVVPPPVGYKMENGQYIPDENAHFIKEIFEAYAAGEGARHIAQRLAAQGCLTKRGNPIDNRFVDYVLHNPVYIGKLRWSVNSHAASSRHYDSADIIVFDGTHEPLISSELWESVQKRLHEVKTLYPKYQRREQPVSFMLKGLVRCSSCGSTLCYCRTSEPSLQCHSYARGSCRQSHSINIATANEAVIKGLQLAVDKLDFAIAPAKPHYSADAPGTNKLLAAEYKKMERIKAAYANGTDTLEEYAANKKKISAEIARLEAELQQESNVKPINKKAFAKRVSEIIKYISDPHNSEAAKNQALRTVISYIIFDRAATTFNIIFHF

SEQ ID NO: 7 - protein, 550 aa, synthetic construct
  REGION 1..550: note = human gut metagenome
MKIAIYARKSKYSPTGESVENQIQLCKEYLQAKYKSETLEIDEYKDEGYSGGNTNRPDFKKLIAQIEDYDMLICYRLDRISRNVADFSSTLTLLQNNKCDFVSIKEQFDTTSPMGRAMIYISSVFAQLERETIAERIRDNMMELAKMGRWLGGTIPMGFDSEPITFIDENMKERSMTKLIPNVEELKVIELIYEKYLQLGSMGKVVTYLLQNNIKTKKGKDFTLGSIKVILTNPIYVKANQEVVNHLKTQGITICGDVDGKKALLTYNKTTGISNDVGTKTIVKDKSEWIAAVANHKGIIPADKWLQAQNIKDKNKDSFPALGRSNTTIASRVLRCDKCESTMGVTHGHINPVTGKKHYYYNCTLKKRSKGVRCDNKPAKAAEVDEAILITLENMFKAKSSIIDNLKAKNKARRIEMISSNRVDVINKIIEDKTKQIDNLVNKLSLDDDLTDILFKKIKGLKAEIKELEDELLTLTSDNIKLNEDEVVLDFTEKLLEKCSIIRTLDILEQQQIVDALIPLVTWNGDTEVLNIYPLGSPELELKEAESKKK

SEQ ID NO: 8 - protein, 493 aa, synthetic construct
  REGION 1..493: note = human gut metagenome
MKEKVSERKTGAIYIRVSTDKQEELSPDAQLRLLLDYAKKDSIDVPKEYIFQDNGISGRKANKRPAFQNMIALAKSKEHPIDTIIVWKFSRFARNQEESIVYKSLLKKNNVDVVSVSEPLIDGPFGSLIERIIEWMDEYYSIRLSGEVMRGMTQNAMRGHYQSDAPIGYTSPGDKKPPVINPDTVQIPLMIKDMFLSGSTQLQIARKLNDSGYRTKRGNLWDARGVRYVLENPFYIGKSRWNYTERGRRLKPADEVIYADGNWEALWDEDTFKEIQKRLALNMRKSKSRDISAAKHWLSGLLICSSCGGTLAFGGAHNMRGFQCWKYSKGFCSESHYISTGPIEKMVLEYLEAVMHSPALSYTVISSSSVDASSKLSDLERQLQKIDAKEKRIKAAYLNEIDTLEEYKANKTALEEERRTVEKEIEELTLSDVKYSKEDLDKKMKQNISDLLRVLRDESADYIQKGNMMRNVVDHIVFNRKNTSLDVFLKLVV

SEQ ID NO: 9 - protein, 404 aa, synthetic construct
  REGION 1..404: note = human gut metagenome
MKITKKQPLRPRGRSEDKRQSTKNVIRDAYINGPQKEVQIIPAKRDMEAETEKKKLRVCAYCRVSTDEDTQASSYELQVQNYTRMIRENPEWEFAGIFADEGISGTSVLHREHFLEMIEKCKAGEIDLIITKQVSRFARNVLDSLNYIFMLRKLDPPVGVYFETEKLNTLDKSSDMVITVLSLVAQSESEQKSNSLKWSFKRRRAQGLGIYPSWALLGYRLDDEKNWEIVEDEADIVRTIYSLYLDGYSSTQIAELLTKSGIPTVKGLSVWSSGSVLGILKNEKFCGDALCQKTVTIDFFTHKSVKNNGIEPQYFVEGHHIPIIEKNDWLLAQQIRKERRYRKRRSTHRKPRIVVKGALSGFMIVDTSWDEEYVDSLLISATQKPEPAPVIAEEDENFIVIEKE

SEQ ID NO: 10 - protein, 498 aa, synthetic construct
  REGION 1..498: note = human gut metagenome
MADIQPVKNGALYIRVSTHLQEELSPDAQKRLLMEYAEAHNIIVLKEHIYIDSGISGRSARQRPQFNNMIAEAKSKEHPFDVILVWKYSRFARNQEESIVYKSMLKRENVDVISVSEPISDDPFGSLIERIIEWMDEYYSIRLSGEVSRGMAENAMRGNYQARPPLGYRIPGYRQTPVIVPEEAELIQLIFDLYTEKKMGIFEIVRYLNEHGYQTGHKKPFQRRSVTYILKNPTYIGKTIWNQHDQDHKLRDKSEWIIADGKHEPIISKEQFDKAQKRIESTYKPAYRKPTSVCHHWLSSLLKCSSCGRTLVVKRTASKKKDRMYVNFQCYGYQKGICNTNQSISAIKLEPVIMHALEDAMTSGKIHFDVLNPTTLDSSQKQQFLTRLNEIEKKEERIKRAYRDGIDTLEEYKENKSIIQTEKEMLLKKIEHIEEPALSPEEAKPIMMDRIKNVYEIITNPDIGMEEKNKAARSIIEKIVFDRATGSVNIFFYLAHCP

SEQ ID NO: 11 - protein, 500 aa, synthetic construct [sequence omitted]

SEQ ID NO: 12 - protein, 485 aa, synthetic construct
  REGION 1..485: note = Mycobacterium phage Bxb2
MTKKVAIYTRVSTTNQAEEGFSIDEQIDRLTKYAEAMGWQVSDTYTDAGFSGAKLERPAMQRLINDIENKAFDTVLVYKLDRLSRSVRDTLYLVKDVFTKNKIDFISLNESIDTSSAMGSLFLTILSAINEFERENIKERMTMGKLGRAKSGKSMMWTKTAFGYYHNRKTGILEIVPLQATIVEQIFTDYLSGISLTKLRDKLNESGHIGKDIPWSYRTLRQTLDNPVYCGYIKFKDSLFEGMHKPIIPYETYLKVQKELEERQQQTYERNNNPRPFQAKYMLSGMARCGYCGAPLKIVLGHKRKDGSRTMKYHCANRFPRKTKGITVYNDNKKCDSGTYDLSNLENTVIDNLIGFQENNDSLLKIINGNNQPILDTSSFKKQISQIDKKIQKNSDLYLNDFITMDELKDRTDSLQAEKKLLKAKISENKFNDSTDVFELVKTQLGSIPINELSYDNKKKIVNNLVSKVDVTADNVDIIFKFQLA

SEQ ID NO: 13 - protein, 594 aa, synthetic construct
  REGION 1..594: note = Streptomyces virus phiBT1
MSPFIAPDVPEHLLDTVRVFLYARQSKGRSDGSDVSTEAQLAAGRALVASRNAQGGARWVVAGEFVDVGRSGWDPNVTRADFERMMGEVRAGEGDVVVVNELSRLTRKGAHDALEIDNELKKHGVRFMSVLEPFLDTSTPIGVAIFALIAALAKQDSDLKAERLKGAKDEIAALGGVHSSSAPFGMRAVRKKVDNLVISVLEPDEDNPDHVELVERMAKMSFEGVSDNAIATTFEKEKIPSPGMAERRATEKRLASIKARRLNGAEKPIMWRAQTVRWILNHPAIGGFAFERVKHGKAHINVIRRDPGGKPLTPHTGILSGSKWLELQEKRSGKNLSDRKPGAEVEPTLLSGWRFLGCRICGGSMGQSQGGRKRNGDLAEGNYMCANPKGHGGLSVKRSELDEFVASKVWARLRTADMEDEHDQAWIAAAAERFALQHDLAGVADERREQQAHLDNVRRSIKDLQADRKAGLYVGREELETWRSTVLQYRSYEAECTTRLAELDEKMNGSTRVPSEWFSGEDPTAEGGIWASWDVYERREFLSFFLDSVMVDRGRHPETKKYIPLKDRVTLKWAELLKEEDEASEATERELAAL

SEQ ID NO: 14 - protein, 528 aa, synthetic construct
  REGION 1..528: note = Bacillus cereus AH187
MYPYDVPDYAGSYRPESLDVCIYLRKSRKDVEEERRAIEEGSSYNALERHRKRLFAIAKAENHNIIDIFEEVASGESIQERPQMQQLLRKLEGNEIDGVLVIDLDRLGRGDMLDAGMIDRAFRYSSTKIITPTDVYDPDDESWELVFGIKSLISRQELKSITKRLQNGRIDSVKEGKHIGKKPPYGYLKDENLRLYPDPEKAWIVKKIFELMCDGKGRQMIAAELDRLGIDPPVTKRGAWDSSTITSIIKNEVYTGVIVWGKFKHKKRNGKYTRHKNPQEKWIMYENAHEPIISKELFDAANEAHSSRHKPAVITSKKLTNPLAGILKCKLCGYTMLIQTRKDRPHNYLRCNNPACKGKQKQSVFNLVEEKLLYSLQQIVDEYQAQKVEEVEIDDSKLISFKEKAIISKEKELKELQAQKGNLHDLLEQGIYTVEIFLERQKNLVERITSIENDIEVLQKEIETEQIKEHNKTEFIPALKTVIESYHKTTNIELKNQLLKTILSTVTYYRHPDWKTNEFEIQVYFKIS

SEQ ID NO: 15 - protein, 486 aa, synthetic construct
  REGION 1..486: note = Bacillus cytotoxicus NVH 391?98
MYPYDVPDYAGSAVGIYIRVSTQEQASEGHSIESQKKKLASYCEIQGWDDYRFYIEEGISGKNTNRPKLKLLMEHIEKGKINILLVYRLDRLTRSVIDLHKLLNFLQEHGCAFKSATETYDTTTANGRMSMGIVSLLAQWETENMSERIKLNLEHKVLVEGERVGAIPYGFDLSDDEKLVKNEKSAILLDMVERVENGWSVNRIVNYLNLTNNDRNWSPNGVLRLLRNPALYGATRWNDKIAENTHEGIISKERFNRLQQILADRSIHHRRDVKGTYIFQGVLRCPVCDQTLSVNRFIKKRKDGTEYCGVLYRCQPCIKQNKYNLAIGEARFLKALNEYMSTVEFQTVEDEVIPKKSEREMLESQLQQIARKREKYQKAWASDLMSDDEFEKLMVETRETYDECKQKLESCEDPIKIDETYLKEIVYMFHQTFNDLESEKQKEFISKFIRTIRYTVKEQQPIRPDKSKTGKGKQKVIITEVEFYQS

SEQ ID NO: 16 - protein, 472 aa, synthetic construct
  REGION 1..472: note = Staphylococcus lugdunensis N920143
MYPYDVPDYAGSKVAIYTRVSSAEQANEGYSIHEQKKKLISYCEIHDWNEYKVFTDAGISGGSMKRPALQKLMKHLSSFDLVLVYKLDRLTRNVRDLLDMLEEFEQYNVSFKSATEVFDTTSAIGKLFITMVGAMAEWERETIRERSLFGSRAAVREGNYIREAPFCYDNIEGKLHPNEYAKVIDLIVSMFKKGISANEIARRLNSSKVHVPNKKSWNRNSLIRLMRSPVLRGHTKYGDMLIENTHEPVLSEHDYNAINNAISSKTHKSKVKHHAIFRGALVCPQCNRRLHLYAGTVKDRKGYKYDVRRYKCETCSKNKDVKNVSFNESEVENKFVNLLKSYELNKFHIRKVEPVKKIEYDIDKINKQKINYTRSWSLGYIEDDEYFELMEEINATKKMIEEQTTENKQSVSKEQIQSINNFILKGWEELTIKDKEELILSTVDKIEFNFIPKDKKHKTNTLDINNIHFKFS

SEQ ID NO: 17 - DNA, 49 bp, synthetic construct
  misc_feature 1..49: note = AttB nucleic acid sequence
  source 1..49: mol_type = other DNA
tgtctactatgtctttatgccacatgtgtcgcatatacagatagtagac

SEQ ID NO: 18 - DNA, 50 bp, synthetic construct
  misc_feature 1..50: note = AttP nucleic acid sequence
  source 1..50: mol_type = other DNA
tccattgcgagtgctaatgatgcttgtgtcgccatggcagagcacattgc

SEQ ID NO: 19 - DNA, 50 bp, synthetic construct
  misc_feature 1..50: note = AttB nucleic acid sequence
  source 1..50: mol_type = other DNA
ggccgcgaggtcgtgttcgtcgtcatgttgaggttcacgaccatcacgcc

SEQ ID NO: 20 - DNA, 50 bp, synthetic construct
  misc_feature 1..50: note = AttP nucleic acid sequence
  source 1..50: mol_type = other DNA
atggagccgttctccgcggacgtcatggactacggcgtgatcggtttgaa

SEQ ID NO: 21 - DNA, 50 bp, synthetic construct
  misc_feature 1..50: note = AttB nucleic acid sequence
  source 1..50: mol_type = other DNA
cattatatgtttttacaatccgggccgccatactgtaagaacatataatg

SEQ ID NO: 22 - DNA, 50 bp, synthetic construct
  misc_feature 1..50: note = AttP nucleic acid sequence
  source 1..50: mol_type = other DNA
gctgccgctgcctcaccatctgggccgccatactggcttataataaactg

SEQ ID NO: 23 - DNA, 50 bp, synthetic construct
  misc_feature 1..50: note = AttB nucleic acid sequence
  source 1..50: mol_type = other DNA
tataaactgatataattcaaagttataacttgatatattcaagatgtaga

SEQ ID NO: 24 - DNA, 50 bp, synthetic construct
  misc_feature 1..50: note = AttP nucleic acid sequence
  source 1..50: mol_type = other DNA
atttgaacctgtagttggtgctttataaatgcgtaactaataattcatat

SEQ ID NO: 25 - DNA, 50 bp, synthetic construct
  misc_feature 1..50: note = AttB nucleic acid sequence
  source 1..50: mol_type = other DNA
aatgaggtcagacgcatggagcgccgcctccgcatgcgtcagggtcgatg

SEQ ID NO: 26 - DNA, 50 bp, synthetic construct
  misc_feature 1..50: note = AttP nucleic acid sequence
  source 1..50: mol_type = other DNA
tacagcgctataataaagtagcgccgcgacgccattgccgcagagcttgc

SEQ ID NO: 27 - DNA, 50 bp, synthetic construct
  misc_feature 1..50: note = AttB nucleic acid sequence
  source 1..50: mol_type = other DNA
tattatatctaaaagcagtatggcggagcttagtgcttttagatataatt

SEQ ID NO: 28 - DNA, 50 bp, synthetic construct
  misc_feature 1..50: note = AttP nucleic acid sequence
  source 1..50: mol_type = other DNA
aagctcattataagtcagtacggcggccccgacggcgagctcggcgcttc

SEQ ID NO: 29 - DNA, 50 bp, synthetic construct
  misc_feature 1..50: note = AttB nucleic acid sequence
  source 1..50: mol_type = other DNA
tgtatcattttcatatagtgtgcaggtgctaactatatgaaaatgataca

SEQ ID NO: 30 - DNA, 50 bp, synthetic construct
  misc_feature 1..50: note = AttP nucleic acid sequence
  source 1..50: mol_type = other DNA
aactacgaagcattgcttgatgcaggtgctaattttgcatcttcccccag

SEQ ID NO: 31 - DNA, 50 bp, synthetic construct
  misc_feature 1..50: note = AttB nucleic acid sequence
  source 1..50: mol_type = other DNA
cgttatagggtattgcagtaccgaccgccatactgtaataccctataacg

SEQ ID NO: 32 - DNA, 50 bp, synthetic construct
  misc_feature 1..50: note = AttP nucleic acid sequence
  source 1..50: mol_type = other DNA
gatgcactgagctcaccgtccggaccgccatactgacttatgatataaga

SEQ ID NO: 33 - DNA, 50 bp, synthetic construct
  misc_feature 1..50: note = AttB nucleic acid sequence
  source 1..50: mol_type = other DNA
gtttataaaaccgatgccgctttgacagaagcggaacgggttttaataag

SEQ ID NO: 34 - DNA, 50 bp, synthetic construct
  misc_feature 1..50: note = AttP nucleic acid sequence
  source 1..50: mol_type = other DNA
acgatattgcctgcaaaagtgcagacagaaacgaggaacagaaaaatggt

SEQ ID NO: 35 - DNA, 50 bp, synthetic construct
  misc_feature 1..50: note = AttB nucleic acid sequence
  source 1..50: mol_type = other DNA
gttagtacccaaatgataaaaggatgaccttttgtcatttgggtactaac

SEQ ID NO: 36 - DNA, 50 bp, synthetic construct
  misc_feature 1..50: note = AttP nucleic acid sequence
  source 1..50: mol_type = other DNA
acagggtctcttgcccgaactggatgacacaatggggatcaaagtactta

SEQ ID NO: 37 - DNA, 46 bp, synthetic construct
  misc_feature 1..46: note = attB site sequence
  source 1..46: mol_type = other DNA
ggccggcttgtcgacgacggcggtctccgtcgtcaggatcatccgg

SEQ ID NO: 38 - DNA, 52 bp, synthetic construct
  misc_feature 1..52: note = AttP nucleic acid sequence
  source 1..52: mol_type = other DNA
gtggtttgtctggtcaaccaccgcggtctcagtggtgtacggtacaaaccca

SEQ ID NO: 39 - DNA, 31 bp, synthetic construct
  misc_feature 1..31: note = AttB nucleic acid sequence
  source 1..31: mol_type = other DNA
cacaattaacatctcaatcaaggtaaatgct

SEQ ID NO: 40 - DNA, 50 bp, synthetic construct
  misc_feature 1..50: note = AttP nucleic acid sequence
  source 1..50: mol_type = other DNA
gcgagtttttatttcgtttatttcaattaaggtaactaaaaaactccttt

SEQ ID NO: 41 - DNA, 34 bp, synthetic construct
  misc_feature 1..34: note = AttB nucleic acid sequence
  source 1..34: mol_type = other DNA
caggtttttgacgaaagtgatccagatgatccag

SEQ ID NO: 42 - DNA, 57 bp, synthetic construct
  misc_feature 1..57: note = AttP nucleic acid sequence
  source 1..57: mol_type = other DNA
ttcgggtgctgggttgttgtctctggacagtgatccatgggaaactactcagcacca

SEQ ID NO: 43 - DNA, 62 bp, synthetic construct
  misc_feature 1..62: note = AttB nucleic acid sequence
  source 1..62: mol_type = other DNA
gtaatatgtttggatatggggaagtgaatcagtacaaccgccacagtaccctcatgtcagcc

SEQ ID NO: 44 - DNA, 62 bp, synthetic construct
  misc_feature 1..62: note = AttP nucleic acid sequence
  source 1..62: mol_type = other DNA
ataatagtgtatatggtagagaattaaaccagtttaatactccaccatgtacacgcagtgag

SEQ ID NO: 45 - DNA, 65 bp, synthetic construct
  misc_feature 1..65: note = AttB nucleic acid sequence
  source 1..65: mol_type = other DNA
cgcatacattgttgttgtttttccagatccagttggtcctgtaaatataagcaatccatgtgagt

SEQ ID NO: 46 - DNA, 64 bp, synthetic construct
  misc_feature 1..64: note = AttP nucleic acid sequence
  source 1..64: mol_type = other DNA
caataacggttgtatttgtagaacttgaccagttgttttagtaacataaatacaactccgaata

SEQ ID NO: 47 - DNA, 64 bp, synthetic construct; identical to SEQ ID NO: 46
  misc_feature 1..64: note = AttB nucleic acid sequence
  source 1..64: mol_type = other DNA

SEQ ID NO: 48 - DNA, 65 bp, synthetic construct
  misc_feature 1..65: note = AttP nucleic acid sequence
  source 1..65: mol_type = other DNA
tttttgtatgttagttgtgtcactgggtagacctaaatagtgacacaactgctattaaaatttaa

SEQ ID NO: 49 - DNA, 1446 bp, synthetic construct
  misc_feature 1..1446: note = SscINT
  source 1..1446: mol_type = other DNA
atgaatgagaagaaccttgagataggggctgcatacattcgggtcagcaccgacgaccagactgaactgtctcccgatgctcagctgcgggtaatccttgaggcggccaagaaagacgggattataattcctcaggagttcgtgttcatggaggacagaggccgttccggccgccgggctgataacagacctgagttccagaggatgatttccaccgctcgacagaatccttctccattcaggtatttatacctttggaagttcagtcggttcgcaagaaatcaggaggaatcagctttctacaagggaattctgcggaaaaagtgcggcgtgacgatcaaatctgttagtgagcccattatggagggcatgttcgggcgcttggtagaaatgatcatcgaatggtctgatgaattctacagcgttaacctcagcggtgaagtcctcaggggaatgacgcaaaaggcattagagcatggataccagttaaccccctgcctgggctacgatgctgtgggacatggaagaccgtacgtcatcaacgaggagcagtatcagattgttgaatttatccaccgcagctttttcgatggtaaggatatgacgtggattgctagggaagctaacagaaggggatatcacactcgcagggggaatccattcgataccagggcagtgagaatcatcctgaccaattctttctatgtgggactcgtgaaatggaacgacgtaacatttcaaggcacacatgagtgccgggaaagcgtgacttctgtattctccgcgaatcaggaaaggctgaatcgtattcaccgaccaagggggcggcgacaggcctcttcctgtaaacactggctgagcggcctcctgaagtgctcaatatgcggagctagtctgggctacaaccagaccaaagacctgacaaagcgaggtcatgctttccagtgctggaagtacaccaaaggaattcatcctggctcttgcagcgtatcctctctcaaagcagaggcggccgttctggagtccctgcaaatgatattggaaactggagaggtcgagtatacctacgaacagcgcgagaagcacctggatgataacaaactcaccctcatccagaagtccttggaacgacttgacaccaaagagctgcgaattcgagaggcttacgagtctggaatagataccttggatgagttcaagacaaataaggcacgactgcagcgagagcgtgatcaactcatggaagagcttgaagaattgcactctcaagaggagccagaggatgtccccggcaaggagatcttaatcgaacgtattcaaaatgtatacgatttgctgcaatccccagatgtcgataatgatgataaaggcaacgccgtgcggtcaattatcaagaagatagtgtatattaaggaatctaaaactttctgtttttattattatgtg

SEQ ID NO: 50 - DNA, 1479 bp, synthetic construct
  misc_feature 1..1479: note = SacINT
  source 1..1479: mol_type = other DNA
atgaaggagaaggtgagtgagagaaaaacaggcgccatttacataagagtttctacggacaaacaggaagagctttcaccagacgcacagctgaggctcctcctggactacgctaagaaagattctatcgatgttcctaaggagtacatcttccaagataacggcattagtgggcgaaaagcgaacaagcgccccgcgttccagaatatgatcgcactcgcgaagtccaaagagcacccaatcgacacaatcattgtgtggaagttctctcgctttgcccggaatcaggaggaatcaattgtgtacaagagtttactcaaaaaaaacaacgtcgatgtggtgagtgtgtccgagcctctgattgatgggccatttggaagcctgattgagagaattattgagtggatggacgagtattattccattcgattgtctggcgaggtgatgcgtggtatgactcaaaatgccatgcgggggcattaccagagcgatgcaccgattgggtacacatccccaggggacaaaaagcccccggttataaacccggataccgttcagattcctctgatgatcaaagatatgttcttaagcggctcaacccagctgcaaattgccagaaagctcaacgacagtggctataggacaaagcgcggtaacctgtgggacgcgagaggcgtccggtacgtcctggagaacccgttttacatcgggaaaagccgctggaattacacggagagagggcgacggctgaagccggcagatgaggtgatatacgctgacgggaactgggaggcactgtgggatgaggacaccttcaaggagatccaaaaaagattggcactgaatatgcgcaagtccaagtctagggacatctcagctgcaaaacactggctgagcggtctcttaatctgttcttcctgcggcggaaccctggccttcgggggagcacacaatatgagggggtttcaatgctggaaatactcaaaggggttctgcagcgaatcccattatatcagcaccggtccaattgagaaaatggttctggagtacttagaggccgtcatgcactcccctgcgctgagttacacggttatcagtagttcatccgtcgatgccagctccaaactgtcagacctggagcgccaattgcagaaaatagacgccaaggagaaacgcatcaaggcagcatacctcaacgaaatagatacactggaggagtacaaagctaataaaacagccttggaggaagaacgccgtaccgtcgagaaggaaatcgaggagctcaccctcagcgacgtgaaatattctaaggaggaccttgacaagaaaatgaagcagaatatatcagacctgctgcgggtgctgagagacgaatctgccgattacatccagaaaggtaacatgatgagaaacgtggtcgatcatatcgtctttaacaggaagaatactagcctggacgtttttctgaaattagtagtg

SEQ ID NO: 51 - DNA, 1582 bp, synthetic construct
  misc_feature 1..1582: note = BceINT
  source 1..1582: mol_type = other DNA
tacccttatgacgtacctgattacgccggtagctacaggccagaatccctcgacgtatgcatttaccttcgcaaatccaggaaggacgttgaagaagaacgccgcgcaatcgaagaaggcagctcctacaacgcactggaacggcatcggaagcgattgtttgccattgccaaggcagaaaatcacaacatcatcgatatttttgaagaagttgccagtggagagagcatacaggaaagaccccaaatgcagcagctgctcaggaagttggaaggcaatgaaattgatggcgtgctggtgattgatctcgatagactcgggcggggcgatatgctggatgcgggaatgatcgatcgtgcattcagatactcatctaccaaaattatcaccccaacagatgtctacgatcctgatgacgaaagttgggagctggtgttcgggattaagagtttaatcagccgacaggagctcaagtccatcaccaaacgactgcagaatggccggatcgattcagtgaaggaggggaagcacattggcaagaagccaccttatggctacttgaaggatgagaatctgaggctgtatccagatccagaaaaggcctggattgtgaagaagatttttgaactgatgtgtgacggaaagggacggcagatgattgcggctgagttggacagactgggtattgacccccctgtgacgaaaaggggagcatgggactctagtaccatcaccagtattataaagaacgaagtttatacaggcgtcattgtctgggggaaatttaagcataaaaagaggaatggtaagtatacgcggcataagaacccacaggagaagtggattatgtacgagaacgcccatgaacccattatatccaaagagcttttcgatgcggcaaacgaagcccatagctccagacacaagcccgctgtcataacgagtaaaaagctgactaacccactggctggcatcttgaagtgcaagttgtgtggctacacaatgctcatacagactcggaaggacaggcctcataactacttacgatgtaacaatccagcctgtaagggcaagcaaaaacagtcagttttcaatttagtggaggagaagttgctctattcactgcagcaaatcgtggacgagtaccaggcccagaaagttgaagaggtcgaaattgatgattctaaactcatctcttttaaggaaaaggcaataatctccaaagagaaggagcttaaggagttacaagctcagaaaggcaacctgcatgacctgctcgaacaaggtatttacacggtcgaaatcttcctggaacggcagaagaatttggtggaaagaataaccagcatcgagaacgacatcgaggtgctgcagaaggagattgaaactgagcagatcaaagaacacaataagaccgagttcatccccgccttaaaaacggtgatcgaatcatatcacaaaacaaccaatattgaactcaaaaaccagctgctgaagaccattctgagcaccgtgacatactataggcatcccgactggaaaaccaatgaatttgaaatccaggtgtacttcaaaatctcct

SEQ ID NO: 52 - protein, 1367 aa, synthetic construct
  REGION 1..1367: note = SpCas9
DKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 53 - protein, 575 aa, synthetic construct
  REGION 1..575: note = RT(1-478)-Sto7d
LNIEDEYRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGPPPSHQWYTVLDLKDAFFCLRLHPTSQPLFAFEWRDPEMGISGQLTWTRLPQGFKNSPTLFNEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGKAGFCRLFIPGFAEMAAPLYPLTKPGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDGTGGGGVTVKFKYKGEELEVDISKIKKVWRVGKMISFTYDDNGKTGRGAVSEKDAPKELLQMLEKSGKKSGGSKRTADG

SEQ ID NO: 54 - RNA, 19 nt, synthetic construct
  misc_feature 1..19: note = MS2 Stem-Loop
  source 1..19: mol_type = other RNA
acatgaggatcacccatgt

SEQ ID NO: 55 - DNA, 38 bp, synthetic construct
  misc_feature 1..38: note = Polynucleotide
  source 1..38: mol_type = other DNA
ccccaactggggtaacctttgagttctctcagttgggg

SEQ ID NO: 56 - DNA, 38 bp, synthetic construct
  misc_feature 1..38: note = attB site sequence
  source 1..38: mol_type = other DNA
ggcttgtcgacgacggcgttctccgtcgtcaggatcat

SEQ ID NO: 57 - DNA, 38 bp, synthetic construct
  misc_feature 1..38: note = attB site sequence
  source 1..38: mol_type = other DNA
ggcttgtcgacgacggcgaactccgtcgtcaggatcat

SEQ ID NO: 58 - DNA, 38 bp, synthetic construct
  misc_feature 1..38: note = attB site sequence
  source 1..38: mol_type = other DNA
ggcttgtcgacgacggcgccctccgtcgtcaggatcat

SEQ ID NO: 59 - DNA, 38 bp, synthetic construct
  misc_feature 1..38: note = attB site sequence
  source 1..38: mol_type = other DNA
ggcttgtcgacgacggcgggctccgtcgtcaggatcat

SEQ ID NO: 60 - DNA, 38 bp, synthetic construct
  misc_feature 1..38: note = attB site sequence
  source 1..38: mol_type = other DNA
ggcttgtcgacgacggcgtgctccgtcgtcaggatcat

SEQ ID NO: 61 - DNA, 38 bp, synthetic construct
  misc_feature 1..38: note = attB site sequence
  source 1..38: mol_type = other DNA
ggcttgtcgacgacggcggtctccgtcgtcaggatcat

SEQ ID NO: 62 - DNA, 38 bp, synthetic construct
  misc_feature 1..38: note = attB site sequence
  source 1..38: mol_type = other DNA
ggcttgtcgacgacggcgctctccgtcgtcaggatcat

SEQ ID NO: 63 - DNA, 38 bp, synthetic construct
  misc_feature 1..38: note = attB site sequence
  source 1..38: mol_type = other DNA
ggcttgtcgacgacggcgcactccgtcgtcaggatcat

SEQ ID NO: 64 - DNA, 38 bp, synthetic construct
  misc_feature 1..38: note = attB site sequence
  source 1..38: mol_type = other DNA
ggcttgtcgacgacggcgtcctccgtcgtcaggatcat

SEQ ID NO: 65 - DNA, 38 bp, synthetic construct
  misc_feature 1..38: note = attB site sequence
  source 1..38: mol_type = other DNA
ggcttgtcgacgacggcggactccgtcgtcaggatcat

SEQ ID NO: 66 - DNA, 38 bp, synthetic construct
  misc_feature 1..38: note = attB site sequence
  source 1..38: mol_type = other DNA
ggcttgtcgacgacggcgagctccgtcgtcaggatcat

SEQ ID NO: 67 - DNA, 38 bp, synthetic construct
  misc_feature 1..38: note = attB site sequence
  source 1..38: mol_type = other DNA
ggcttgtcgacgacggcgacctccgtcgtcaggatcat

SEQ ID NO: 68 - DNA, 38 bp, synthetic construct
  misc_feature 1..38: note = attB site sequence
  source 1..38: mol_type = other DNA
ggcttgtcgacgacggcgatctccgtcgtcaggatcat

SEQ ID NO: 69 - DNA, 38 bp, synthetic construct
  misc_feature 1..38: note = attB site sequence
  source 1..38: mol_type = other DNA
ggcttgtcgacgacggcggcctccgtcgtcaggatcat

SEQ ID NO: 70 - DNA, 38 bp, synthetic construct
  misc_feature 1..38: note = attB site sequence
  source 1..38: mol_type = other DNA
ggcttgtcgacgacggcgcgctccgtcgtcaggatcat

SEQ ID NO: 71 - DNA, 38 bp, synthetic construct
  misc_feature 1..38: note = attB site sequence
  source 1..38: mol_type = other DNA
ggcttgtcgacgacggcgtactccgtcgtcaggatcat

SEQ ID NO: 72 - DNA, 52 bp, synthetic construct
  misc_feature 1..52: note = attP site sequence
  source 1..52: mol_type = other DNA
gtggtttgtctggtcaaccaccgcgttctcagtggtgtacggtacaaaccca

SEQ ID NO: 73 - DNA, 52 bp, synthetic construct
  misc_feature 1..52: note = attP site sequence
  source 1..52: mol_type = other DNA
gtggtttgtctggtcaaccaccgcgaactcagtggtgtacggtacaaaccca

SEQ ID NO: 74 - DNA, 52 bp, synthetic construct
  misc_feature 1..52: note = attP site sequence
  source 1..52: mol_type = other DNA
gtggtttgtctggtcaaccaccgcgccctcagtggtgtacggtacaaaccca

SEQ ID NO: 75 - DNA, 52 bp, synthetic construct
  misc_feature 1..52: note = attP site sequence
  source 1..52: mol_type = other DNA
gtggtttgtctggtcaaccaccgcgggctcagtggtgtacggtacaaaccca

SEQ ID NO: 76 - DNA, 52 bp, synthetic construct
  misc_feature 1..52: note = attP site sequence
  source 1..52: mol_type = other DNA
gtggtttgtctggtcaaccaccgcgtgctcagtggtgtacggtacaaaccca

SEQ ID NO: 77 - DNA, 52 bp, synthetic construct
  misc_feature 1..52: note = attP site sequence
  source 1..52: mol_type = other DNA
gtggtttgtctggtcaaccaccgcgctctcagtggtgtacggtacaaaccca

SEQ ID NO: 78 - DNA, 52 bp, synthetic construct
  misc_feature 1..52: note = attP site sequence
  source 1..52: mol_type = other DNA
gtggtttgtctggtcaaccaccgcgcactcagtggtgtacggtacaaaccca

SEQ ID NO: 79 - DNA, 52 bp, synthetic construct
  misc_feature 1..52: note = attP site sequence
  source 1..52: mol_type = other DNA
gtggtttgtctggtcaaccaccgcgtcctcagtggtgtacggtacaaaccca

SEQ ID NO: 80 - DNA, 52 bp, synthetic construct
  misc_feature 1..52: note = attP site sequence
  source 1..52: mol_type = other DNA
gtggtttgtctggtcaaccaccgcggactcagtggtgtacggtacaaaccca

SEQ ID NO: 81 - DNA, 52 bp, synthetic construct
  misc_feature 1..52: note = attP site sequence
  source 1..52: mol_type = other DNA
gtggtttgtctggtcaaccaccgcgagctcagtggtgtacggtacaaaccca

SEQ ID NO: 82 - DNA, 52 bp, synthetic construct
  misc_feature 1..52: note = attP site sequence
  source 1..52: mol_type = other DNA
gtggtttgtctggtcaaccaccgcgacctcagtggtgtacggtacaaaccca

SEQ ID NO: 83 - DNA, 52 bp, synthetic construct
  misc_feature 1..52: note = attP site sequence
  source 1..52: mol_type = other DNA
gtggtttgtctggtcaaccaccgcgatctcagtggtgtacggtacaaaccca

SEQ ID NO: 84 - DNA, 52 bp, synthetic construct
  misc_feature 1..52: note = attP site sequence
  source 1..52: mol_type = other DNA
gtggtttgtctggtcaaccaccgcggcctcagtggtgtacggtacaaaccca

SEQ ID NO: 85 - DNA, 52 bp, synthetic construct
  misc_feature 1..52: note = attP site sequence
  source 1..52: mol_type = other DNA
gtggtttgtctggtcaaccaccgcgcgctcagtggtgtacggtacaaaccca

SEQ ID NO: 86 - DNA, 52 bp, synthetic construct
  misc_feature 1..52: note = attP site sequence
  source 1..52: mol_type = other DNA
gtggtttgtctggtcaaccaccgcgtactcagtggtgtacggtacaaaccca

SEQ ID NO: 87 - DNA, 66 bp, synthetic construct
  misc_feature 1..66: note = Polynucleotide
  source 1..66: mol_type = other DNA
ggaagcggagctactaacttcagcctgctgaagcaggctggcgacgtggaggagaaccctggacct

SEQ ID NO: 88 - DNA, 45 bp, synthetic construct
  misc_feature 1..45: note = Polynucleotide
  source 1..45: mol_type = other DNA
gggggaggaggttctggaggcggaggctccggaggcggagggtca

SEQ ID NO: 89 - DNA, 15 bp, synthetic construct
  misc_feature 1..15: note = Polynucleotide
  source 1..15: mol_type = other DNA
ggaggtggcgggagc

SEQ ID NO: 90 - DNA, 15 bp, synthetic construct
  misc_feature 1..15: note = Polynucleotide
  source 1..15: mol_type = other DNA
cccgcaccagcgcct

SEQ ID NO: 91 - DNA, 45 bp, synthetic construct
  misc_feature 1..45: note = Polynucleotide
  source 1..45: mol_type = other DNA
gaggcagctgccaaggaagccgctgccaaggaggcggccgcaaag

SEQ ID NO: 92 - DNA, 48 bp, synthetic construct
  misc_feature 1..48: note = Polynucleotide
  source 1..48: mol_type = other DNA
agtgggagcgagacccctgggactagcgagtcagctacacccgaaagc

SEQ ID NO: 93 - DNA, 54 bp, synthetic construct
  misc_feature 1..54: note = Polynucleotide
  source 1..54: mol_type = other DNA
ggggggtcaggtggatccggcggaagtggcggatccggtggatctggcggcagt

SEQ ID NO: 94 - DNA, 15 bp, synthetic construct
  misc_feature 1..15: note = Polynucleotide
  source 1..15: mol_type = other DNA
gaagctgctgctaag

SEQ ID NO: 95 - DNA, 78 bp, synthetic construct
  misc_feature 1..78: note = Polynucleotide
  source 1..78: mol_type = other DNA
gctggcagcgagacaccaggaacaagcgagtcagcaacaccagagagcagtggcggcagcagcggcggcagcagcacc

SEQ ID NO: 96 - protein, 22 aa, synthetic construct
  REGION 1..22: note = Polypeptide
GSGATNFSLLKQAGDVEENPGP

SEQ ID NO: 97 - protein, 15 aa, synthetic construct
  REGION 1..15: note = Polypeptide
GGGGSGGGGSGGGGS

SEQ ID NO: 98 - protein, 5 aa, synthetic construct
  REGION 1..5: note = Polypeptide
GGGGS

SEQ ID NO: 99 - protein, 5 aa, synthetic construct
  REGION 1..5: note = Polypeptide
PAPAP

SEQ ID NO: 100 - protein, 15 aa, synthetic construct
  REGION 1..15: note = Polypeptide
EAAAKEAAAKEAAAK

SEQ ID NO: 101 - protein, 16 aa, synthetic construct
  REGION 1..16: note = Polypeptide
SGSETPGTSESATPES

SEQ ID NO: 102 - protein, 18 aa, synthetic construct
  REGION 1..18: note = Polypeptide
GGSGGSGGSGGSGGSGGS

SEQ ID NO: 103 - protein, 5 aa, synthetic construct
  REGION 1..5: note = Polypeptide
EAAAK

SEQ ID NO: 104 - protein, 26 aa, synthetic construct
  REGION 1..26: note = Polypeptide
AGSETPGTSESATPESSGGSSGGSST

SEQ ID NO: 105 - protein, 132 aa, synthetic construct
  REGION 1..132: note = MS2 Coat Protein (MCP)
MASNFTQFVLVDNGGTGDVTVAPSNFANGVAEWISSNSRSQAYKVTCSVRQSSAQKRKYTIKVEVPKVATQTVGGVELPVAAWRSYLNMELTIPIFATNSDCELIVKAMQGLLKDGNPIPSAIAANSGIYSA

SEQ ID NO: 106 - DNA, 396 bp, synthetic construct
  misc_feature 1..396: note = MS2 Coat Protein (MCP)
  source 1..396: mol_type = other DNA
atggcttcaaactttactcagttcgtgctcgtggacaatggtgggacaggggatgtgacagtggctccttctaatttcgctaatggggtggcagagtggatcagctccaactcacggagccaggcctacaaggtgacatgcagcgtcaggcagtctagtgcccagaagagaaagtataccatcaaggtggaggtccccaaagtggctacccagacagtgggcggagtcgaactgcctgtcgccgcttggaggtcctacctgaacatggagctcactatcccaattttcgctaccaattctgactgtgaactcatcgtgaaggcaatgcaggggctcctcaaagacggtaatcctatcccttccgccatcgccgctaactcaggtatctacagcgct

SEQ ID NO: 107 - DNA, 46 bp, synthetic construct
  misc_feature 1..46: note = attP site sequence
  source 1..46: mol_type = other DNA
ccggatgatcctgacgacggagaccgccgtcgtcgacaagccggcc

(19) United States
(12) Patent Application Publication
(10) Pub. No.: US 2023/0287441 A1
(43) Pub. Date: Sep. 14, 2023

(54) PROGRAMMABLE INSERTION APPROACHES VIA REVERSE TRANSCRIPTASE RECRUITMENT

(71) Applicant: Massachusetts Institute of Technology, Cambridge, MA (US)

(72) Inventors: Omar Abudayyeh, Cambridge, MA (US); Jonathan Gootenberg, Cambridge, MA (US); Lukas Villiger, Cambridge, MA (US); Kaiyi Jiang, Cambridge, MA (US)

(21) Appl. No.: 18/067,214

(22) Filed: Dec. 16, 2022

Related U.S. Application Data
(60) Provisional application No. 63/265,661, filed on Dec. 17, 2021.

(51) Int. Cl. C12N 15/82; C07K 14/005; C12N 9/22; C12N 9/12

(52) U.S. Cl. CPC C12N 15/8213; C07K 14/005; C12N 9/22; C12N 9/1276; C12Y 207/07049; C12N 2770/32322

(57) ABSTRACT

This disclosure provides complexes for prime editing comprising an RNA-guided nuclease, a fusion protein comprising a reverse transcriptase domain linked to a nucleic acid binding protein, and a guide RNA (gRNA) comprising at least one protein-recruiting stem-loop nucleic acid sequence, wherein the protein-recruiting stem-loop nucleic acid sequence binds to the nucleic acid binding protein. Also provided are systems, methods, and compositions for site-specific genetic engineering using Programmable Addition via Site-Specific Targeting Elements (PASTE) with integration enzymes paired with the prime editing complex.

### STATEMENT REGARDING FEDERALLY SPONSORED RESEARCH OR DEVELOPMENT

This invention was made with Government support under Grant No. R21 AI149694 awarded by the National Institutes of Health. The Government has certain rights in the invention.

### BACKGROUND

Editing genomes using the RNA-guided DNA targeting principle of CRISPR-Cas (Clustered Regularly Interspaced Short Palindromic Repeats-CRISPR associated proteins) has been widely exploited and has become a powerful genome editing means for a wide variety of applications. A wide range of applications using the CRISPR system have been developed, including the use of additional proteins that confer extra functional properties. However, there exists a need for strategies to recruit these additional proteins to the CRISPR system in the genome.

### SUMMARY

In one aspect, the disclosure provides a complex for genome editing comprising: (i) an RNA-guided nuclease; (ii) a fusion protein comprising a reverse transcriptase domain linked to a nucleic acid binding protein; and (iii) a guide RNA (gRNA) comprising a 5′ end and a 3′ end and comprising at least one protein-recruiting stem-loop nucleic acid sequence, wherein the protein-recruiting stem-loop nucleic acid sequence binds to the nucleic acid binding protein.

In certain embodiments, the nucleic acid binding protein is MS2 coat protein (MCP) or PP7 coat protein.

In certain embodiments, the protein-recruiting stem-loop nucleic acid sequence is a MS2 sequence or PP7 stem loop sequence. In certain embodiments, the MS2 sequence comprises a nucleic acid sequence of ACAUGAGGAUCACCCAUGU. (SEQ ID NO:54)

In certain embodiments, the gRNA comprises a primer binding site (PBS), a reverse transcriptase (RT) template sequence, and an integration site sequence.

In certain embodiments, the gRNA comprises 1, 2, 3, 4, 5, or 6 protein-recruiting stem-loop nucleic acid sequences.

In certain embodiments, the gRNA comprises 2 or more distinct protein-recruiting stem-loop nucleic acid sequences.

In certain embodiments, the protein-recruiting stem-loop nucleic acid sequences are identical.

In certain embodiments, the protein-recruiting stem-loop nucleic acid sequence is present at the 5′ end of the gRNA, the 3′ end of the gRNA, or both. In certain embodiments, the gRNA comprises two protein-recruiting stem-loop nucleic acid sequences present at the 5′ end of the gRNA, the 3′ end of the gRNA, or both.

In certain embodiments, the complex comprises one or more additional gRNAs.

In certain embodiments, the one or more additional gRNAs comprise at least one protein-recruiting stem-loop nucleic acid sequence.

In certain embodiments, the complex comprises two or more gRNAs, each gRNA comprising a different target at desired locations in a cell genome.

In certain embodiments, the RNA-guided nuclease comprises a CRISPR nuclease. In certain embodiments, the CRISPR nuclease is Cas9 or Cas12. In certain embodiments, the CRISPR nuclease comprises nickase activity. In certain embodiments, the CRISPR nuclease is selected from Cas9-D10A, Cas9-H840A, and Cas12a/b nickase.

In certain embodiments, the reverse transcriptase domain is selected from the group consisting of Moloney Murine Leukemia Virus (M-MLV) reverse transcriptase domain, transcription xenopolymerase (RTX), avian myeloblastosis virus reverse transcriptase (AMV-RT), and Eubacterium rectale maturase RT (MarathonRT).

In certain embodiments, the reverse transcriptase domain comprises a mutation relative to the wild-type sequence or contains a stabilization domain like the DNA-binding Sto7d protein from Sulfolobus tokodaii.

In certain embodiments, the M-MLV reverse transcriptase domain comprises one or more mutations selected from the group consisting of D200N, T306K, W313F, T330P, L603W, and L139P.

In certain embodiments, the reverse transcriptase domain is linked to the nucleic acid binding protein via a linker. In certain embodiments, the linker is cleavable. In certain embodiments, the linker is non-cleavable. In certain embodiments, the complex comprises any one or more of the linker sequences recited in Table 4.

In certain embodiments, the one or both of the RNA-guided nuclease and fusion protein are linked to an integration enzyme or fragment thereof (e.g., an integrase or fragment thereof).

In certain embodiments, the RNA-guided nuclease is linked to an integration enzyme or fragment thereof (e.g., an integrase or fragment thereof).

In certain embodiments, the fusion protein is linked to an integration enzyme or fragment thereof (e.g., an integrase or fragment thereof).

In certain embodiments, the integration enzyme is selected from the group consisting of Cre, Dre, Vika, Bxb1, BceINT φC31, RDF, FLP, φBT1, R1, R2, R3, R4, R5, TP901-1, A118, φFC1, φC1, MR11, TG1, φ370.1, Wβ, BL3, SPBc, K38, Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, retrotransposases encoded by R2, L1, Tol2 Tc1, Tc3, Mariner (Himar 1), Mariner (mos 1), and Minos, and any mutants thereof.

In certain embodiments, the integration enzyme is Bxb1 or a mutant thereof.

In certain embodiments, the integration enzyme is BceINT or a mutant thereof.

In certain embodiments, the integration enzyme comprises an amino acid sequence that is at least 90% identical to an amino acid sequence set forth in any one of SEQ ID NOs: 1-16.

In certain embodiments, the integration enzyme recognizes an integration site.

In certain embodiments, the integration site is an attB site, an attP site, an attL site, an attR site, a lox71 site a Vox site, or a FRT site.

In certain embodiments, the integration enzyme recognizes nucleic acid attachment sites attB and attP, other recognition site pairs, or any pseudosites in a human genome.

In certain embodiments, the attB and/or attP nucleic acid sequence is between 12 and 60 nucleotides in length or between 18 and 50 nucleotides in length.

In certain embodiments, the attB and/or attP nucleic acid sequence comprises one or more truncations. In certain embodiments, the attB and/or attP nucleic acid sequence is truncated by 1 to 32 nucleotides from one or both of the 5′ end and 3′ end.

In certain embodiments, the integration enzyme binds to any one of the attB nucleic acid sequences selected from the group consisting of SEQ ID NOs: 17, 19, 21, 23, 25, 27, 29, 31, 33, 35, 37, 39, 41, 43, 45, and 47. In certain embodiments, the integration enzyme binds to any one of the attP nucleic acid sequences selected from the group consisting of SEQ ID NOs: 18, 20, 22, 24, 26, 28, 30, 32, 34, 36, 38, 40, 42, 44, 46, and 48.

In certain embodiments: a) the integrase or fragment thereof comprises an amino acid sequence that is at least 90% identical to an amino acid sequence set forth in SEQ ID NO: 1, wherein the integrase binds to the attB nucleic acid set forth in SEQ ID NO: 17 and the attP nucleic acid set forth in SEQ ID NO: 18; b) the integrase or fragment thereof comprises an amino acid sequence that is at least 90% identical to an amino acid sequence set forth in SEQ ID NO: 2, wherein the integrase binds to the attB nucleic acid set forth in SEQ ID NO: 19 and the attP nucleic acid set forth in SEQ ID NO: 20; c) the integrase or fragment thereof comprises an amino acid sequence that is at least 90% identical to an amino acid sequence set forth in SEQ ID NO: 3, wherein the integrase binds to the attB nucleic acid set forth in SEQ ID NO: 21 and the attP nucleic acid set forth in SEQ ID NO: 22; d) the integrase or fragment thereof comprises an amino acid sequence that is at least 90% identical to an amino acid sequence set forth in SEQ ID NO: 4, wherein the integrase binds to the attB nucleic acid set forth in SEQ ID NO: 23 and the attP nucleic acid set forth in SEQ ID NO: 24; e) the integrase or fragment thereof comprises an amino acid sequence that is at least 90% identical to an amino acid sequence set forth in SEQ ID NO: 5, wherein the integrase binds to the attB nucleic acid set forth in SEQ ID NO: 25 and the attP nucleic acid set forth in SEQ ID NO: 26; f) the integrase or fragment thereof comprises an amino acid sequence that is at least 90% identical to an amino acid sequence set forth in SEQ ID NO: 6, wherein the integrase binds to the attB nucleic acid set forth in SEQ ID NO: 27 and the attP nucleic acid set forth in SEQ ID NO: 28; g) the integrase or fragment thereof comprises an amino acid sequence that is at least 90% identical to an amino acid sequence set forth in SEQ ID NO: 7, wherein the integrase binds to the attB nucleic acid set forth in SEQ ID NO: 29 and the attP nucleic acid set forth in SEQ ID NO: 30; h) the integrase or fragment thereof comprises an amino acid sequence that is at least 90% identical to an amino acid sequence set forth in SEQ ID NO: 8, wherein the integrase binds to the attB nucleic acid set forth in SEQ ID NO: 31 and the attP nucleic acid set forth in SEQ ID NO: 32; i) the integrase or fragment thereof comprises an amino acid sequence that is at least 90% identical to an amino acid sequence set forth in SEQ ID NO: 9, wherein the integrase binds to the attB nucleic acid set forth in SEQ ID NO: 33 and the attP nucleic acid set forth in SEQ ID NO: 34; j) the integrase or fragment thereof comprises an amino acid sequence that is at least 90% identical to an amino acid sequence set forth in SEQ ID NO: 10, wherein the integrase binds to the attB nucleic acid set forth in SEQ ID NO: 35 and the attP nucleic acid set forth in SEQ ID NO: 36; k) the integrase or fragment thereof comprises an amino acid sequence that is at least 90% identical to an amino acid sequence set forth in SEQ ID NO: 11, wherein the integrase binds to the attB nucleic acid set forth in SEQ ID NO: 37 and the attP nucleic acid set forth in SEQ ID NO: 38; l) the integrase or fragment thereof comprises an amino acid sequence that is at least 90% identical to an amino acid sequence set forth in SEQ ID NO: 12, wherein the integrase binds to the attB nucleic acid set forth in SEQ ID NO: 39 and the attP nucleic acid set forth in SEQ ID NO: 40; m) the integrase or fragment thereof comprises an amino acid sequence that is at least 90% identical to an amino acid sequence set forth in SEQ ID NO: 13, wherein the integrase binds to the attB nucleic acid set forth in SEQ ID NO: 41 and the attP nucleic acid set forth in SEQ ID NO: 42; n) the integrase or fragment thereof comprises an amino acid sequence that is at least 90% identical to an amino acid sequence set forth in SEQ ID NO: 14, wherein the integrase binds to the attB nucleic acid set forth in SEQ ID NO: 43 and the attP nucleic acid set forth in SEQ ID NO: 44; o) the integrase or fragment thereof comprises an amino acid sequence that is at least 90% identical to an amino acid sequence set forth in SEQ ID NO: 15, wherein the integrase binds to the attB nucleic acid set forth in SEQ ID NO: 45 and the attP nucleic acid set forth in SEQ ID NO: 46; or p) the integrase or fragment thereof comprises an amino acid sequence that is at least 90% identical to an amino acid sequence set forth in SEQ ID NO: 16, wherein the integrase binds to the attB nucleic acid set forth in SEQ ID NO: 47 and the attP nucleic acid set forth in SEQ ID NO: 48.

In certain embodiments, any one of the attB nucleic acid sequences selected from the group consisting of SEQ ID NOs: 17, 19, 21, 23, 25, 27, 29, 31, 33, 35, 37, 39, 41, 43, 45, and 47 is truncated by 1 to 32 nucleotides from one or both of the 5′ end and 3′ end.

In certain embodiments, any one of the attP nucleic acid sequences selected from the group consisting of SEQ ID NOs: 18, 20, 22, 24, 26, 28, 30, 32, 34, 36, 38, 40, 42, 44, 46, and 48 is truncated by 1 to 32 nucleotides from one or both of the 5′ end and 3′ end.

In certain embodiments, the RNA-guided nuclease interacts with a gRNA comprising a primer binding sequence linked to an integration sequence.

In certain embodiments, the gRNA interacts with the RNA-guided nuclease and targets a desired location in a cell genome.

In certain embodiments, the RNA-guided nuclease nicks a strand of the cell genome and the reverse transcriptase domain incorporates the integration sequence of the gRNA into the nicked site, thereby providing the integration site at the desired location of the cell genome.

In certain embodiments, the integrase is capable of binding the integration sequence.

In one aspect, the disclosure provides a polynucleotide comprising a nucleic acid sequence encoding the RNA-guided nuclease described above.

In one aspect, the disclosure provides a polynucleotide comprising a nucleic acid sequence encoding the gRNA described above.

In one aspect, the disclosure provides a polynucleotide comprising a nucleic acid sequence encoding the fusion protein described above.

In one aspect, the disclosure provides a vector comprising any of the polynucleotides described above.

In one aspect, the disclosure provides a host cell comprising the vector described above.

In one aspect, the disclosure provides a method of site-specific integration of a nucleic acid into a cell genome, the method comprising:
(a) incorporating an integration site at a desired location in the cell genome by introducing into the cell:
   i. an RNA-guided nuclease comprising a nickase activity;
   ii. a fusion protein comprising a reverse transcriptase domain linked to a nucleic acid binding protein; and
   iii. a guide RNA (gRNA) comprising a 5′ end and a 3′ end and comprising a primer binding sequence linked to an integration sequence and at least one protein-recruiting stem-loop nucleic acid sequence, wherein the protein-recruiting stem-loop nucleic acid sequence binds to the nucleic acid binding protein, wherein the gRNA interacts with the RNA-guided nuclease and targets the desired location in the cell genome, wherein the RNA-guided nuclease nicks a strand of the cell genome and the reverse transcriptase domain incorporates the integration sequence of the gRNA into the nicked site, thereby providing the integration site at the desired location of the cell genome; and
(b) integrating the nucleic acid into the cell genome by introducing into the cell:
   i. a DNA or RNA strand comprising the nucleic acid linked to a sequence that is complementary or associated to the integration site; and
   ii. an integration enzyme or fragment thereof, wherein the integration enzyme or fragment thereof incorporates the nucleic acid into the cell genome at the integration site by integration, recombination, or reverse transcription of the sequence that is complementary or associated to the integration site, thereby introducing the nucleic acid into the desired location of the cell genome of the cell.

In certain embodiments, the nucleic acid binding protein is MS2 coat protein (MCP) or PP7 coat protein.

In certain embodiments, the protein-recruiting stem-loop nucleic acid sequence is a MS2 sequence or PP7 stem loop sequence.

In certain embodiments, the MS2 sequence comprises a nucleic acid sequence of ACAUGAGGAUCACCCAUGU. (SEQ ID NO:54)

In certain embodiments, the gRNA comprises 1, 2, 3, 4, 5, or 6 protein-recruiting stem-loop nucleic acid sequences.

In certain embodiments, the gRNA comprises 2 or more distinct protein-recruiting stem-loop nucleic acid sequences.

In certain embodiments, the protein-recruiting stem-loop nucleic acid sequences are identical.

In certain embodiments, the protein-recruiting stem-loop nucleic acid sequence is present at the 5′ end of the gRNA, the 3′ end of the gRNA, or both. In certain embodiments, the gRNA comprises two protein-recruiting stem-loop nucleic acid sequences present at the 5′ end of the gRNA, the 3′ end of the gRNA, or both.

In certain embodiments, the method comprises one or more additional gRNAs. In certain embodiments, the one or more additional gRNAs comprise at least one protein-recruiting stem-loop nucleic acid sequence,

In certain embodiments, the RNA-guided nuclease comprises a CRISPR nuclease. In certain embodiments, the CRISPR nuclease is Cas9 or Cas12. In certain embodiments, the CRISPR nuclease comprises nickase activity. In certain embodiments, the CRISPR nuclease is selected from Cas9-D10A, Cas9-H840A, and Cas12a/b nickase.

In certain embodiments, the reverse transcriptase domain is selected from the group consisting of Moloney Murine Leukemia Virus (M-MLV) reverse transcriptase domain, transcription xenopolymerase (RTX), avian myeloblastosis virus reverse transcriptase (AMV-RT), and Eubacterium rectale maturase RT (MarathonRT).

In certain embodiments, the reverse transcriptase domain comprises a mutation relative to the wild-type sequence or contains a stabilization domain like the DNA-binding Sto7d protein from Sulfolobus tokodaii.

In certain embodiments, the M-MLV reverse transcriptase domain comprises one or more mutations selected from the group consisting of D200N, T306K, W313F, T330P, L603W, and L139P.

In certain embodiments, the reverse transcriptase domain is linked to the nucleic acid binding protein via a linker. In certain embodiments, the linker is cleavable. In certain embodiments, the linker is non-cleavable. In certain embodiments, the linker comprises any one or more of the linker sequences recited in Table 4.

In certain embodiments, the one or both of the RNA-guided nuclease and fusion protein are linked to an integration enzyme or fragment thereof (e.g., an integrase or fragment thereof).

In certain embodiments, the integration enzyme is selected from the group consisting of Cre, Dre, Vika, Bxb1, BceINT φC31, RDF, FLP, φBT1, R1, R2, R3, R4, R5, TP901-1, A118, φFC1, φC1, MR11, TG1, φ370.1, Wβ, BL3, SPBc, K38, Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, retrotransposases encoded by R2, L1, Tol2 Tc1, Tc3, Mariner (Himar 1), Mariner (mos 1), and Minos, and any mutants thereof.

In certain embodiments, the integration enzyme is Bxb1 or a mutant thereof.

In certain embodiments, the integration enzyme is BceINT or a mutant thereof.

In certain embodiments, the integration enzyme comprises an amino acid sequence that is at least 90% identical to an amino acid sequence set forth in any one of SEQ ID NOs: 1-16.

In certain embodiments, the integration enzyme recognizes an integration site.

In certain embodiments, the integration site is an attB site, an attP site, an attL site, an attR site, a lox71 site a Vox site, or a FRT site.

In certain embodiments, the integration enzyme recognizes nucleic acid attachment sites attB and attP, other recognition site pairs, or any pseudosites in a human genome.

In certain embodiments, the attB and/or attP nucleic acid sequence is between 12 and 60 nucleotides in length or between 18 and 50 nucleotides in length.

In certain embodiments, the attB and/or attP nucleic acid sequence comprises one or more truncations. In certain embodiments, the attB and/or attP nucleic acid sequence is truncated by 1 to 32 nucleotides from one or both of the 5′ end and 3′ end.

In certain embodiments, the integration enzyme binds to any one of the attB nucleic acid sequences selected from the group consisting of SEQ ID NOs: 17, 19, 21, 23, 25, 27, 29, 31, 33, 35, 37, 39, 41, 43, 45, and 47.

In certain embodiments, the integration enzyme binds to any one of the attP nucleic acid sequences selected from the group consisting of SEQ ID NOs: 18, 20, 22, 24, 26, 28, 30, 32, 34, 36, 38, 40, 42, 44, 46, and 48.

In certain embodiments, the: a) the integrase or fragment thereof comprises an amino acid sequence that is at least 90% identical to an amino acid sequence set forth in SEQ ID NO: 1, wherein the integrase binds to the attB nucleic acid set forth in SEQ ID NO: 17 and the attP nucleic acid set forth in SEQ ID NO: 18; b) the integrase or fragment thereof comprises an amino acid sequence that is at least 90% identical to an amino acid sequence set forth in SEQ ID NO: 2, wherein the integrase binds to the attB nucleic acid set forth in SEQ ID NO: 19 and the attP nucleic acid set forth in SEQ ID NO: 20; c) the integrase or fragment thereof comprises an amino acid sequence that is at least 90% identical to an amino acid sequence set forth in SEQ ID NO: 3, wherein the integrase binds to the attB nucleic acid set forth in SEQ ID NO: 21 and the attP nucleic acid set forth in SEQ ID NO: 22; d) the integrase or fragment thereof comprises an amino acid sequence that is at least 90% identical to an amino acid sequence set forth in SEQ ID NO: 4, wherein the integrase binds to the attB nucleic acid set forth in SEQ ID NO: 23 and the attP nucleic acid set forth in SEQ ID NO: 24; e) the integrase or fragment thereof comprises an amino acid sequence that is at least 90% identical to an amino acid sequence set forth in SEQ ID NO: 5, wherein the integrase binds to the attB nucleic acid set forth in SEQ ID NO: 25 and the attP nucleic acid set forth in SEQ ID NO: 26; f) the integrase or fragment thereof comprises an amino acid sequence that is at least 90% identical to an amino acid sequence set forth in SEQ ID NO: 6, wherein the integrase binds to the attB nucleic acid set forth in SEQ ID NO: 27 and the attP nucleic acid set forth in SEQ ID NO: 28; g) the integrase or fragment thereof comprises an amino acid sequence that is at least 90% identical to an amino acid sequence set forth in SEQ ID NO: 7, wherein the integrase binds to the attB nucleic acid set forth in SEQ ID NO: 29 and the attP nucleic acid set forth in SEQ ID NO: 30; h) the integrase or fragment thereof comprises an amino acid sequence that is at least 90% identical to an amino acid sequence set forth in SEQ ID NO: 8, wherein the integrase binds to the attB nucleic acid set forth in SEQ ID NO: 31 and the attP nucleic acid set forth in SEQ ID NO: 32; i) the integrase or fragment thereof comprises an amino acid sequence that is at least 90% identical to an amino acid sequence set forth in SEQ ID NO: 9, wherein the integrase binds to the attB nucleic acid set forth in SEQ ID NO: 33 and the attP nucleic acid set forth in SEQ ID NO: 34; j) the integrase or fragment thereof comprises an amino acid sequence that is at least 90% identical to an amino acid sequence set forth in SEQ ID NO: 10, wherein the integrase binds to the attB nucleic acid set forth in SEQ ID NO: 35 and the attP nucleic acid set forth in SEQ ID NO: 36; k) the integrase or fragment thereof comprises an amino acid sequence that is at least 90% identical to an amino acid sequence set forth in SEQ ID NO: 11, wherein the integrase binds to the attB nucleic acid set forth in SEQ ID NO: 37 and the attP nucleic acid set forth in SEQ ID NO: 38; 1) the integrase or fragment thereof comprises an amino acid sequence that is at least 90% identical to an amino acid sequence set forth in SEQ ID NO: 12, wherein the integrase binds to the attB nucleic acid set forth in SEQ ID NO: 39 and the attP nucleic acid set forth in SEQ ID NO: 40; m) the integrase or fragment thereof comprises an amino acid sequence that is at least 90% identical to an amino acid sequence set forth in SEQ ID NO: 13, wherein the integrase binds to the attB nucleic acid set forth in SEQ ID NO: 41 and the attP nucleic acid set forth in SEQ ID NO: 42; n) the integrase or fragment thereof comprises an amino acid sequence that is at least 90% identical to an amino acid sequence set forth in SEQ ID NO: 14, wherein the integrase binds to the attB nucleic acid set forth in SEQ ID NO: 43 and the attP nucleic acid set forth in SEQ ID NO: 44; o) the integrase or fragment thereof comprises an amino acid sequence that is at least 90% identical to an amino acid sequence set forth in SEQ ID NO: 15, wherein the integrase binds to the attB nucleic acid set forth in SEQ ID NO: 45 and the attP nucleic acid set forth in SEQ ID NO: 46; or p) the integrase or fragment thereof comprises an amino acid sequence that is at least 90% identical to an amino acid sequence set forth in SEQ ID NO: 16, wherein the integrase binds to the attB nucleic acid set forth in SEQ ID NO: 47 and the attP nucleic acid set forth in SEQ ID NO: 48.

In certain embodiments, any one of the attB nucleic acid sequences selected from the group consisting of SEQ ID NOs: 17, 19, 21, 23, 25, 27, 29, 31, 33, 35, 37, 39, 41, 43, 45, and 47 is truncated by 1 to 32 nucleotides from one or both of the 5′ end and 3′ end.

In certain embodiments, any one of the attP nucleic acid sequences selected from the group consisting of SEQ ID NOs: 18, 20, 22, 24, 26, 28, 30, 32, 34, 36, 38, 40, 42, 44, 46, and 48 is truncated by 1 to 32 nucleotides from one or both of the 5′ end and 3′ end.

### BRIEF DESCRIPTION OF THE DRAWINGS

Aspects, features, benefits and advantages of the embodiments described herein will be apparent with regard to the following description, appended claims, and accompanying drawings.

FIG. 1 shows a schematic diagram of a concept of Programmable Addition via Site-Specific Targeting Elements (PASTE) according to embodiments of the present teachings.

FIG. 2 shows a schematic representation of using Bxb1 to integrate a nucleic acid into the genome according to embodiments of the present teachings.

FIG. 3 shows the percent integration of GFP or Gluc into the attB locus using Bxb1 Programmable Addition via Site-Specific Targeting Elements (PASTE) according to embodiments of the present teachings.

FIG. 4 shows the percent editing of various HEK3 targeting pegRNA Programmable Addition via Site-Specific Targeting Elements (PASTE) according to embodiments of the present teachings.

FIG. 5A-FIG. 5C shows a schematic of the integrase discovery pipeline from bacterial and metagenomic sequences (FIG. 5A) and the phylogenetic tree of discovered integrases showing distinct subfamilies (FIG. 5B and FIG. 5C).

FIG. 6A-FIG. 6I show the activity of several integrases. FIG. 6A shows an Integrase integration activity screen using reporters in HEK293FT cells compared to BxbINT and phiC31a. FIG. 6B shows PASTE integration activity with the most active integrases compared to BxbINT. FIG. 6C shows a characterization of integrase integration activity with truncated attachment sites using reporters in HEK293FT cells. FIG. 6D shows PASTE integration activity with BceINT and BcyINT with truncated attachment sites compared to BxbINT. FIG. 6E shows PASTE integration activity with SscINT and SacINT with truncated attachment sites compared to BxbINT. FIG. 6F shows optimization BceINT and SacINT PASTE constructs via protein fusions for different sized attachment sites compared to BxbINT-based PASTE for EGFP integration at the ACTB locus. FIG. 6G shows BceINT and INT2 PASTE protein constructs compared to BxbINT for EGFP integration at the ACTB locus. FIG. 6H shows integration of EGFP at different endogenous genes for PASTE with either BceINT or BxbINT. FIG. 6I shows PASTE integration activity with various integrases of EGFP at the ACTB locus.

FIG. 7A-FIG. 7F show indirect recruitment of reverse transcriptases via RNA-based recruitment. FIG. 7A shows a schematic diagram of pegRNA modified with MS2 hairpins interacting with MS2-coat protein (MCP) fused to Murine Leukemia Virus (MLV) reverse transcriptase (RT). FIG. 7B and FIG. 7C show comparisons of physically separate nucleases and reverse transcriptases with physically fused PE2 prime editors. FIG. 7D further shows comparisons of editing efficiency at endogenous loci of Cas9-RT fusions and MS2-MCP RNA-based recruitment of reverse transcriptase. FIG. 7E and FIG. 7F show integration efficiency of different iterations of PASTE with RNA-based recruited reverse transcriptases.

### DETAILED DESCRIPTION

It will be appreciated that for clarity, the following discussion will describe various aspects of embodiments of the applicant's teachings. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present disclosure. Thus, appearances of the phrases “in one embodiment,” “in an embodiment,” or “an example embodiment” in various places throughout this specification are not necessarily all referring to the same embodiment, but may. Furthermore, the particular features, structures, or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the disclosure. For example, in the appended claims, any of the claimed embodiments can be used in any combination.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

As used herein, the singular forms “a”, “an,” and “the” include both singular and plural forms unless the context clearly dictates otherwise. Thus, for example, reference to “a cell” includes a plurality of such cells.

As used herein, the term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, the term “about” or “approximately” refers to a measurable value such as a parameter, an amount, a temporal duration, and the like, are meant to encompass variations of and from the specified value, such as variations of +/−10% or less, +/−5% or less, +/−1% or less, +/−0.5% or less, and +/−0.1% or less of and from the specified value, insofar such variations are appropriate to perform in the disclosure. It is to be understood that the value to which the modifier “about” or “approximately” refers is itself also specifically disclosed.

It is noted that all publications and references cited herein are expressly incorporated herein by reference in their entirety. The publications discussed herein are provided solely for their disclosure prior to the filing date of the present application. Nothing herein is to be construed as an admission that the present disclosure is not entitled to antedate such publication. Further, the dates of publication provided may be different from the actual publication dates, which may need to be independently confirmed.

### Overview

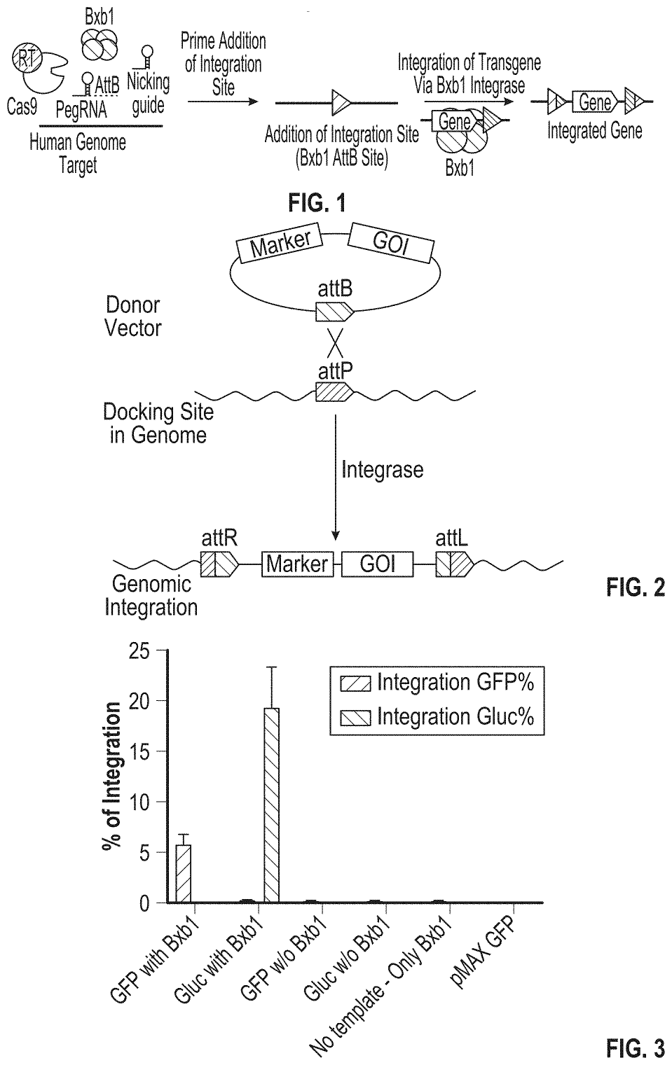

The embodiments disclosed herein provide non-naturally occurring or engineered systems, methods, and compositions for site-specific genetic engineering using Programmable Addition via Site-Specific Targeting Elements (PASTE). A schematic diagram illustrating the concept of PASTE is shown in FIG. 1. As discussed in more details below, the PASTE comprises the addition of an integration site into a target genome followed by the insertion of one or more genes of interest or one or more nucleic acid sequences of interest at the site. This process can be done as one or more reactions into a cell. The addition of the integration site into the target genome is done using gene editing technologies that include for example, without limitation, prime editing, recombinant adeno-associated virus (rAAV)-mediated nucleic acid integration, transcription activator-like effector nucleases (TALENS), and zinc finger nucleases (ZFNs). The integration of the transgene at the integration site is done using integrase technologies that include for example, without limitation, integrases, recombinases and reverse transcriptases. The necessary components for the site-specific genetic engineering disclosed herein comprise at least one or more nucleases, one or more guide RNA (gRNA), one or more integration enzymes, and one or more sequences that are complementary or associated to the integration site and linked to the one or more genes of interest or one or more nucleic acid sequences of interest to be inserted into the cell genome.

An advantage of the non-naturally occurring or engineered systems, methods, and compositions for site-specific genetic engineering disclosed herein is programmable insertion of large elements without reliance on DNA damage responses.

Another advantage of the non-naturally occurring or engineered systems, methods, and compositions for site-specific genetic engineering disclosed herein is facile multiplexing, enabling programmable insertion at multiple sites.

Yet another advantage of the non-naturally occurring or engineered systems, methods, and compositions for site-specific genetic engineering disclosed herein is scalable production and delivery through minicircle templates.

### Prime Editing

The present disclosure provides non-naturally occurring or engineered systems, methods, and compositions for site-specific genetic engineering using gene editing technologies such as prime editing to add an integration site into a target genome. Prime editing will be discussed in more detail below.

Prime editing is a versatile and precise genome editing method that directly writes new genetic information into a specified DNA site. Such method is explained fully in the literature. See, e.g., Anzalone, A. V., et al. “Search-and-replace genome editing without double-strand breaks or donor DNA,” Nature 576, 149-157 (2019). Prime editing uses a catalytically-impaired Cas9 endonuclease that is fused to an engineered reverse transcriptase (RT) (e.g., RNA-dependent DNA polymerase) and programmed with a prime-editing guide RNA (pegRNA). The skilled person in the art would appreciate that the pegRNA both specifies the target site and encodes the desired edit. The catalytically-impaired Cas9 endonuclease also comprises a Cas9 nickase that is fused to the reverse transcriptase. During genetic editing, the Cas9 nickase part of the protein is guided to the DNA target site by the pegRNA. The reverse transcriptase domain then uses the pegRNA to template reverse transcription of the desired edit, directly polymerizing DNA onto the nicked target DNA strand. The edited DNA strand replaces the original DNA strand, creating a heteroduplex containing one edited strand and one unedited strand. Afterward, the prime editor (PE) guides resolution of the heteroduplex to favor copying the edit onto the unedited strand, completing the process.

The prime editors refer to a Moloney Murine Leukemia Virus (M-MLV) reverse transcriptase (RT) fused to a Cas9 H840A nickase. Fusing the RT to the C-terminus of the Cas9 nickase may result in higher editing efficiency. Such a complex is called PE1. The Cas9(H840A) can also be linked to a non-M-MLV reverse transcriptase such as a AMV-RT or XRT (Cas9(H840A)-AMV-RT or XRT). In some embodiments, Cas 9(H840A) can be replaced with Cas12a/b or Cas9(D10A). A Cas9 (wild type), Cas9(H840A), Cas9(D10A) or Cas 12a/b nickase fused to a pentamutant of M-MLV RT (D200N/L603W/T330P/T306K/W313F), having up to about 45-fold higher efficiency is called PE2. In some embodiments, the M-MLV RT comprise one or more of the mutations Y8H, P51L, S56A, S67R, E69K, V129P, L139P, T197A, H204R, V223H, T246E, N249D, E286R, Q2911, E302K, E302R, F309N, M320L, P330E, L435G, L435R, N454K, D524A, D524G, D524N, E562Q, D583N, H594Q, E607K, D653N, and L671P. In some embodiments, the reverse transcriptase can also be a wild-type or modified transcription xenopolymerase (RTX), avian myeloblastosis virus reverse transcriptase (AMV-RT), Feline Immunodeficiency Virus reverse transcriptase (FIV-RT), FeLV-RT (Feline leukemia virus reverse transcriptase), HIV-RT (Human Immunodeficiency Virus reverse transcriptase), or Eubacterium rectale maturase RT (MarathonRT). PE3 involves nicking the non-edited strand, potentially causing the cell to remake that strand using the edited strand as the template to induce HR. The nicking of the non-edited strand can involve the use of a nicking guide RNA (ngRNA).

In certain embodiments, the reverse transcriptase contains a stabilization domain. In certain embodiments, the stabilization domain comprises the DNA-binding Sto7d protein from Sulfolobus tokodaii or the DNA-binding Sso7d protein. The DNA-binding proteins improves processivity and resistance to inhibitors of M-MuLV reverse transcriptase. The DNA-binding Sto7d protein from Sulfolobus tokodaii or the DNA-binding Sso7d protein are described in further detail in Oscorbin et al. (FEBS Letters. 594(24): 4338-4356. 2020), incorporated herein by reference.

Nicking the non-edited strand can increase editing efficiency. For example, nicking the non-edited strand can increase editing efficiency by about 1.1 fold, about 1.3 fold, about 1.5 fold, about 1.7 fold, about 1.9 fold, about 2.1 fold, about 2.3 fold, about 2.5 fold, about 2.7 fold, about 2.9 fold, about 3.1 fold, about 3.3 fold, about 3.5 fold, about 3.7 fold, about 3.9 fold, 4.1 fold, about 4.3 fold, about 4.5 fold, about 4.7 fold, about 4.9 fold, or any range that is formed from any two of those values as endpoints.

Although the optimal nicking position varies depending on the genomic site, nicks positioned 3′ of the edit about 40-90 bp from the pegRNA-induced nick can generally increase editing efficiency without excess indel formation. The prime editing practice allows starting with non-edited strand nicks about 50 bp from the pegRNA-mediated nick, and testing alternative nick locations if indel frequencies exceed acceptable levels.

As used herein, the term “guide RNA” (gRNA) and the like refer to an RNA that guides the insertion or deletion of one or more genes of interest or one or more nucleic acid sequences of interest into a target genome. The gRNA can also refer to a prime editing guide RNA (pegRNA), a nicking guide RNA (ngRNA), and a single guide RNA (sgRNA). In some embodiments, the term “gRNA molecule” refers to a nucleic acid encoding a gRNA. In some embodiments, the gRNA molecule is naturally occurring. In some embodiments, a gRNA molecule is non-naturally occurring. In some embodiments, a gRNA molecule is a synthetic gRNA molecule. A gRNA can target a nuclease or a nickase such as Cas9, Cas 12a/b Cas9(H840A) or Cas9 (D10A) molecule to a target nucleic acid or sequence in a genome. In some embodiments, the gRNA can bind to a DNA nickase bound to a reverse transcriptase domain. A “modified gRNA,” as used herein, refers to a gRNA molecule that has an improved half-life after being introduced into a cell as compared to a non-modified gRNA molecule after being introduced into a cell. In some embodiments, the guide RNA can facilitate the addition of the insertion site sequence for recognition by integrases, transposases, or recombinases.

As used herein, the term “prime-editing guide RNA” (pegRNA) and the like refer to an extended single guide RNA (sgRNA) comprising a primer binding site (PBS), a reverse transcriptase (RT) template sequence, and an integration site sequence that can be recognized by recombinases, integrases, or transposases. For example, the PBS can have a length of at least about 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, or more nt. For example, the PBS can have a length of about 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, or any range that is formed from any two of those values as endpoints. For example, the RT template sequence can have a length of at least about 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt, 36 nt, 37 nt, 38 nt, 39 nt, 40 nt, 41 nt, 42 nt, 43 nt, 44 nt, 45 nt, 46 nt, 47 nt, 48 nt, 49 nt, 50 nt, or more nt. For example, the RT template sequence can have a length of about 4 nt, 5 nt, 6 nt, 7 nt, 8 nt, 9 nt, 10 nt, 11 nt, 12 nt, 13 nt, 14 nt, 15 nt, 16 nt, 17 nt, 18 nt, 19 nt, 20 nt, 21 nt, 22 nt, 23 nt, 24 nt, 25 nt, 26 nt, 27 nt, 28 nt, 29 nt, 30 nt, 31 nt, 32 nt, 33 nt, 34 nt, 35 nt, 36 nt, 37 nt, 38 nt, 39 nt, 40 nt, 41 nt, 42 nt, 43 nt, 44 nt, 45 nt, 46 nt, 47 nt, 48 nt, 49 nt, 50 nt, or any range that is formed from any two of those values as endpoints.

During genome editing, the primer binding site allows the 3′ end of the nicked DNA strand to hybridize to the pegRNA, while the RT template serves as a template for the synthesis of edited genetic information. The pegRNA is capable for instance, without limitation, of (i) identifying the target nucleotide sequence to be edited and (ii) encoding new genetic information that replaces the targeted sequence. In some embodiments, the pegRNA is capable of (i) identifying the target nucleotide sequence to be edited and (ii) encoding an integration site that replaces the targeted sequence.

As used herein, the term “nicking guide RNA” (ngRNA) and the like refer to an RNA sequence that can nick a strand such as an edited strand and a non-edited strand. The ngRNA can induce nicks at about 1 or more nt away from the site of the gRNA-induced nick. For example, the ngRNA can nick at least at about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, or more nt away from the site of the gRNA induced nick. As used herein, the terms “reverse transcriptase” and “reverse transcriptase domain” refer to an enzyme or an enzymatically active domain that can reverse a RNA transcribe into a complementary DNA. The reverse transcriptase or reverse transcriptase domain is a RNA dependent DNA polymerase. Such reverse transcriptase domains encompass, but are not limited, to a M-MLV reverse transcriptase, or a modified reverse transcriptase such as, without limitation, Superscript® reverse transcriptase (Invitrogen; Carlsbad, Calif.), Superscript® VILO™ cDNA synthesis (Invitrogen; Carlsbad, Calif.), RTX, AMV-RT, and Quantiscript Reverse Transcriptase (Qiagen, Hilden, Germany).

The pegRNA-PE complex disclosed herein recognizes the target site in the genome and the Cas9 for example nicks a protospacer adjacent motif (PAM) strand. The primer binding site (PBS) in the pegRNA hybridizes to the PAM strand. The RT template operably linked to the PBS, containing the edit sequence, directs the reverse transcription of the RT template to DNA into the target site. Equilibration between the edited 3′ flap and the unedited 5′ flap, cellular 5′ flap cleavage and ligation, and DNA repair results in stably edited DNA. To optimize base editing, a Cas9 nickase can be used to nick the non-edited strand, thereby directing DNA repair to that strand, using the edited strand as a template.

Prime editing is described in more detail in WO2020191234 and WO2020191248, each of which is incorporated herein by reference.

Integrase Technologies

The present disclosure provides non-naturally occurring or engineered systems, methods, and compositions for site-specific genetic engineering using integrase technologies. Integrase technologies will be discussed in more detail below.

The integrase technologies used herein comprise proteins or nucleic acids encoding the proteins that direct integration of a gene of interest or nucleic acid sequence of interest into an integration site via a nuclease such as a prime editing nuclease. In certain embodiments, the protein directing the integration can be an enzyme such as an integration enzyme. In certain embodiments, the integration enzyme can be an integrase that incorporates the genome or nucleic acid of interest into the cell genome at the integration site by integration. The integration enzyme can be a recombinase that incorporates the genome or nucleic acid of interest into the cell genome at the integration site by recombination. The integration enzyme can be a reverse transcriptase that incorporates the genome or nucleic acid of interest into the cell genome at the integration site by reverse transcription. The integration enzyme can be a retrotransposase that incorporates the genome or nucleic acid of interest into the cell genome at the integration site by retrotransposition.

As used herein, the term “integration enzyme” refers to an enzyme or protein used to integrate a gene of interest or nucleic acid sequence of interest into a desired location or at the integration site, in the genome of a cell, in a single reaction or multiple reactions. Non-limiting examples of integration enzymes include for example, without limitation, Cre, Dre, Vika, Bxb1, φC31, RDF, FLP, φBT1, R1, R2, R3, R4, R5, TP901-1, A118, φFC1, φC1, MR11, TG1, φ370.1, Wβ, BL3, SPBc, K38, Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, and retrotransposases encoded by R2, L1, Tol2 Tc1, Tc3, Mariner (Himar 1), Mariner (mos 1), and Minos. In some embodiments, the term “integration enzyme” refers to a nucleic acid (DNA or RNA) encoding the above-mentioned enzymes. In certain embodiments, the integration enzyme comprises an amino acid sequence that is at least 90% identical to an amino acid sequence set forth in any one of SEQ ID NOs: 1-16. In certain embodiments, the integration enzyme comprises an amino acid sequence that is about 90% identical, about 91% identical, about 92% identical, about 93% identical, about 94% identical, about 95% identical, about 96% identical, about 97% identical, about 98% identical, about 99% identical, or 100% identical to an amino acid sequence set forth in any one of SEQ ID NOs: 1-16.

Integration enzyme fragments are also envisioned. Integration enzyme fragments comprise (e.g., retain) integrase activity.

In certain embodiments, the integration enzyme further comprises one or more mutations. Mutations include, but are not limited to, amino acid substitutions, amino acid deletions, and amino acid insertions.

In some embodiments, the serine integrase φC31 from φC31 phage is used as an integration enzyme. The integrase φC31 in combination with a pegRNA can be used to insert the pseudo attP integration site (CCCCAACTGGGGTAACCTTTGAGTTCTCTCAGTTGGGG) (SEQ ID NO:55). A DNA minicircle containing a gene or nucleic acid of interest and attB (GGCCGGCTTGTCGACGACGGCGGTCTCCGTCGTCAGGATCATCCGG)(SEQ ID NO:37) site can be used to integrate the gene or nucleic acid of interest into the genome of a cell. This integration can be aided by a co-transfection of an expression vector having the φC31 integrase.

As used herein, the term “integrase” refers to a bacteriophage derived integrase, including wild-type integrase and any of a variety of mutant or modified integrases. As used herein, the term “integrase complex” may refer to a complex comprising integrase and integration host factor (IF). As used herein, the term “integrase complex” and the like may also refer to a complex comprising an integrase, an integration host factor, and a bacteriophage X-derived excisionase.

As used herein, the term “recombinase” and the like refer to a site-specific enzyme that mediates the recombination of DNA between recombinase recognition sequences, which results in the excision, integration, inversion, or exchange (e.g., translocation) of DNA fragments between the recombinase recognition sequences. Recombinases can be classified into two distinct families: serine recombinases (e.g., resolvases and invertases) and tyrosine recombinases (e.g., integrases). Examples of serine recombinases include, without limitation, Hin, Gin, Tn3, β-six, CinH, ParA, γ6, Bxb1, φC31, TP901, TG1, φBT1, R1, R2, R3, R4, R5, φRV1, φFC1, MR11, A118, U153, and gp29. Examples of serine recombinases also include, without limitation, recombinases Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, and BxZ2 from Mycobacterial phages. Examples of tyrosine recombinases include, without limitation, Cre, FLP, R, Lambda, HK101, HK022, and pSAM2. The serine and tyrosine recombinase names stem from the conserved nucleophilic amino acid residue that the recombinase uses to attack the DNA and which becomes covalently linked to the DNA during strand exchange.

Recombinases have numerous applications, including the creation of gene knockouts/knock-ins and gene therapy applications. See, e.g., Brown et al., “Serine recombinases as tools for genome engineering.” Methods, 2011; 53(4):372-9; Hirano et al., “Site-specific recombinases as tools for heterologous gene integration.” Appl. Microbiol. Biotechnol. 2011; 92(2):227-39; Chavez and Calos, “Therapeutic applications of the ΦC31 integrase system.” Curr. Gene Ther. 2011; 11(5):375-81; Turan and Bode, “Site-specific recombinases: from tag-and-target-to tag-and-exchange-based genomic modifications.” FASEB J. 2011; 25(12):4088-107; Venken and Bellen, “Genome-wide manipulations of Drosophila melanogaster with transposons, Flp recombinase, and ΦC31 integrase.” Methods Mol. Biol. 2012; 859:203-28; Murphy, “Phage recombinases and their applications.” Adv. Virus Res. 2012; 83:367-414; Zhang et al., “Conditional gene manipulation: Creating a new biological era.” J. Zhejiang Univ. Sci. B. 2012; 13(7):511-24; Karpenshif and Bernstein, “From yeast to mammals: recent advances in genetic control of homologous recombination.” DNA Repair (Amst). 2012; 1; 11(10):781-8; the entire contents of each are hereby incorporated by reference in their entirety.

The recombinases provided herein are not meant to be exclusive examples of recombinases that can be used in embodiments of the disclosure. The methods and compositions of the disclosure can be expanded by mining databases for new orthogonal recombinases or designing synthetic recombinases with defined DNA specificities (See, e.g., Groth et al., “Phage integrases: biology and applications.” J. Mol. Biol. 2004; 335, 667-678; Gordley et al., “Synthesis of programmable integrases.” Proc. Natl. Acad. Sci. USA. 2009; 106, 5053-5058; the entire contents of each are hereby incorporated by reference in their entirety).

Other examples of recombinases that are useful in the systems, methods, and compositions described herein are known to those of skill in the art, and any new recombinase that is discovered or generated is expected to be able to be used in the different embodiments of the disclosure.

As used herein, the term “retrotransposase” and the like refer to an enzyme, or combination of one or more enzymes, wherein at least one enzyme has a reverse transcriptase domain. Retrotransposases are capable of inserting long sequences (e.g., over 3000 nucleotides) of heterologous nucleic acid into a genome. Examples of retrotransposases include for example, without limitation, retrotransposases encoded by elements such as R2, L1, Tol2 Tc1, Tc3, Mariner (Himar 1), Mariner (mos 1), Minos, and any mutants thereof.

As used here, the terms “retrotransposons,” “jumping genes,” “jumping nucleic acids,” and the like refer to cellular movable genetic elements dependent on reverse transcription. The retrotransposons are of non-replication competent cellular origin, and are capable of carrying a foreign nucleic acid sequence. The retrotransposons can act as parasites of retroviruses, retaining certain classical hallmarks, such as long terminal repeats (LTR), retroviral primer binding sites, and the like. However, the naturally occurring retrotransposons usually do not contain functional retroviral structure genes, which would normally be capable of recombining to yield replication competent viruses. Some retrotransposons are examples of so-called “selfish DNA”, or genetic information, which encodes nothing except the ability to replicate itself. The retrotransposon may do so by utilizing the occasional presence of a retrovirus or a retrotransposase within the host cell, efficiently packaging itself within the viral particle, which transports it to the new host genome, where it is expressed again as RNA. The information encoded within that RNA is potentially transported with the jumping gene. A retrotransposon can be a DNA transposon or a retrotransposon, including a LTR retrotransposon or a non-LTR retrotransposon.

Non-long terminal repeat (LTR) retrotransposons are a type of mobile genetic elements that are widespread in eukaryotic genomes. They include two classes: the apurinic/apyrimidinic endonuclease (APE)-type and the restriction enzyme-like endonuclease (RLE)-type. The APE class retrotransposons are comprised of two functional domains: an endonuclease/DNA binding domain, and a reverse transcriptase domain. The RLE class are comprised of three functional domains: a DNA binding domain, a reverse transcription domain, and an endonuclease domain. The reverse transcriptase domain of non-LTR retrotransposon functions by binding an RNA sequence template and reverse transcribing it into the host genome's target DNA. The RNA sequence template has a 3′ untranslated region which is specifically bound to the transposase, and a variable 5′ region generally having Open Reading Frame(s) (“ORF”) encoding transposase proteins. The RNA sequence template may also comprise a 5′ untranslated region which specifically binds the retrotransposase. In some embodiments, a non-LTR transposons can include a LINE retrotransposon, such as L1, and a SINE retrotransposon, such as an Alu sequence. Other examples include for example, without limitation, R1, R2, R3, R4, and R5 retro-transposons (Moss, W. N. et al., RNA Biol. 2011, 8(5), 714-718; and Burke, W. D. et al., Molecular Biology and Evolution 2003, 20(8), 1260-1270). The transposon can be autonomous or non-autonomous.

LTR retrotransposons, which include retroviruses, make up a significant fraction of the typical mammalian genome, comprising about 8% of the human genome and 10% of the mouse genome. Lander et al., 2001, Nature 409, 860-921; Waterson et al., 2002, Nature 420, 520-562. LTR elements include retrotransposons, endogenous retroviruses (ERVs), and repeat elements with HERV origins, such as SINE-R. LTR retrotransposons include two LTR sequences that flank a region encoding two enzymes: integrase and retrotransposase.

ERVs include human endogenous retroviruses (HERVs), the remnants of ancient germ-cell infections. While most HERV proviruses have undergone extensive deletions and mutations, some have retained ORFS coding for functional proteins, including the glycosylated env protein. The env gene confers the potential for LTR elements to spread between cells and individuals. Indeed, all three open reading frames (pol, gag, and env) have been identified in humans, and evidence suggests that ERVs are active in the germline. See, e.g., Wang et al., 2010, Genome Res. 20, 19-27. Moreover, a few families, including the HERV-K (HML-2) group, have been shown to form viral particles, and an apparently intact provirus has recently been discovered in a small fraction of the human population. See, e.g., Bannert and Kurth, 2006, Proc. Natl. Acad. USA 101, 14572-14579.

LTR retrotransposons insert into new sites in the genome using the same steps of DNA cleavage and DNA strand-transfer observed in DNA transposons. In contrast to DNA transposons, however, recombination of LTR retrotransposons involves an RNA intermediate. LTR retrotransposons make up about 8% of the human genome. See, e.g., Lander et al., 2001, Nature 409, 860-921; Hua-Van et al., 2011, Biol. Dir. 6, 19.

### Integration Site

The present disclosure provides non-naturally occurring or engineered systems, methods, and compositions for site-specific genetic engineering via the addition of an integration site into a target genome. The integration site will be discussed in more details below.

As used herein, the term “integration site” refers to the site within the target genome where one or more genes of interest or one or more nucleic acid sequences of interest are inserted.

The integration site can be inserted into the genome or a fragment thereof of a cell using a nuclease, a gRNA, and/or an integration enzyme. The integration site can be inserted into the genome of a cell using a prime editor such as, without limitation, PE1, PE2, and PE3, wherein the integration site is carried on a pegRNA. The pegRNA can target any site that is known in the art. Examples of cites targeted by the pegRNA include, without limitation, ACTB, SUPT16H, SRRM2, NOLC1, DEPDC4, NES, LMNB1, AAVS1 locus, CC10, CFTR, SERPINA1, ABCA4, and any derivatives thereof. The complementary integration site may be operably linked to a gene of interest or nucleic acid sequence of interest in an exogenous DNA or RNA. In some embodiments, one integration site is added to a target genome. In some embodiments, more than one integration sites are added to a target genome.

To insert multiple genes or nucleic acids of interest, two or more integration sites are added to a desired location. Multiple DNA comprising nucleic acid sequences of interest are flanked orthogonal to the integration sequences such as, without limitation, attB, attP, other recognition site pairs, or any pseudosites in the human genome. As used herein, a “pseudosite” is a nucleic acid sequence in the target genome (e.g., a human genome) that is similar to a wild type attB or attP sequences. The sequence similarity is sufficient to allow integration of a nucleic acid sequence with an integrase enzyme. An integration site is “orthogonal” when it does not significantly recognize the recognition site or nucleotide sequence of a recombinase. Thus, one attB site of a recombinase can be orthogonal to an attB site of a different recombinase. In addition, one pair of attB and attP sites of a recombinase can be orthogonal to another pair of attB and attP sites recognized by the same recombinase. A pair of recombinases are considered orthogonal to each other, as defined herein, when there is recognition of each other's attB or attP site sequences. In certain embodiments, the attB nucleic acid sequences selected from the group consisting of SEQ ID NOs: 17, 19, 21, 23, 25, 27, 29, 31, 33, 35, 37, 39, 41, 43, 45, and 47. In certain embodiments, the attP nucleic acid sequences selected from the group consisting of SEQ ID NOs: 18, 20, 22, 24, 26, 28, 30, 32, 34, 36, 38, 40, 42, 44, 46, and 48. In certain embodiments, the attB/attP nucleic acid pair is selected from the group consisting of: SEQ ID NO: 17/SEQ ID NO: 18, SEQ ID NO: 19/SEQ ID NO: 20, SEQ ID NO: 21/SEQ ID NO: 22, SEQ ID NO: 23/SEQ ID NO: 24, SEQ ID NO: 25/SEQ ID NO: 26, SEQ ID NO: 27/SEQ ID NO: 28, SEQ ID NO: 29/SEQ ID NO: 30, SEQ ID NO: 31/SEQ ID NO: 32, SEQ ID NO: 33/SEQ ID NO: 34, SEQ ID NO: 35/SEQ ID NO: 36, SEQ ID NO: 37/SEQ ID NO: 38, SEQ ID NO: 39/SEQ ID NO: 40, SEQ ID NO: 41/SEQ ID NO: 42, SEQ ID NO: 43/SEQ ID NO: 44, SEQ ID NO: 45/SEQ ID NO: 46, and SEQ ID NO: 47/SEQ ID NO: 48.

In certain embodiments, the attB nucleic acid sequence is between 12 and 60 nucleotides in length or between 18 and 50 nucleotides in length. In certain embodiments, the attB nucleic acid sequence is 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides in length.

In certain embodiments, the attP nucleic acid sequence is between 12 and 60 nucleotides in length or between 18 and 50 nucleotides in length. In certain embodiments, the attP nucleic acid sequence is 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides in length.

In certain embodiments, the attB and/or attP nucleic acid sequence comprises one or more truncations. The truncation may be at the 5′ end, 3′end, or both. The truncations to the attB and/or attP nucleic acids sequences may be made while still retaining the ability to bind an integrase.

In certain embodiments, the attB and/or attP nucleic acid sequence is truncated by 1 to 32 nucleotides from one or both of the 5′ end and 3′ end. In certain embodiments, the attB nucleic acid sequence is truncated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 nucleotides from one or both of the 5′ end and 3′ end. In certain embodiments, the attP nucleic acid sequence is truncated by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 nucleotides from one or both of the 5′ end and 3′ end.

In certain embodiments, any one of the attB nucleic acid sequences selected from the group consisting of SEQ ID NOs: 17, 19, 21, 23, 25, 27, 29, 31, 33, 35, 37, 39, 41, 43, 45, and 47 is truncated by 1 to 32 nucleotides from one or both of the 5′ end and 3′ end. In certain embodiments, any one of the attP nucleic acid sequences selected from the group consisting of SEQ ID NOs: 18, 20, 22, 24, 26, 28, 30, 32, 34, 36, 38, 40, 42, 44, 46, and 48 is truncated by 1 to 32 nucleotides from one or both of the 5′ end and 3′ end.

The lack of recognition of integration sites can be less than about 30%. In some embodiments, the lack of recognition of integration sites or pairs of sites can be less than about 30%, less than about 28%, less than about 26%, less than about 24%, less than about 22%, less than about 20%, less than about 18%, less than about 16%, less than about 14%, less than about 12%, less than about 10%, less than about 8%, less than about 6%, less than about 4%, less than about 2%, about 1%, or any range that is formed from any two of those values as endpoints. The crosstalk can be less than about 30%. In some embodiments, the crosstalk is less than about 30%, less than about 28%, less than about 26%, less than about 24%, less than about 22%, less than about 20%, less than about 18%, less than about 16%, less than about 14%, less than about 12%, less than about 10%, less than about 8%, less than about 6%, less than about 4%, less than about 2%, less than about 1%, or any range that is formed from any two of those values as endpoints.

In some embodiments, the attB and/or attP site sequences comprise a central dinucleotide sequence. It has been shown that, for example, the central dinucleotide can be changed to GA from GT and that only GA containing attB/attP sites interact and will not cross react with GT containing sequences. In some embodiments, the central dinucleotide is selected from the group consisting of AG, AC, TG, TC, CA, CT, GA, AA, TT, CC, GG, AT, TA, GC, CG and GT.

As used herein, the term “pair of an attB and attP site sequences” and the like refer to attB and attP site sequences that share the same central dinucleotide and can recombine. This means that in the presence of one serine integrase as many as six pairs of these orthogonal att sites can recombine (attPTT will specifically recombine with attBTT, attPTC will specifically recombine with attBTC, and so on).

In some embodiments, the central dinucleotide is nonpalindromic. In some embodiments, the central dinucleotide is palindromic. In some embodiments, a pair of an attB site sequence and an attP site sequence are used in different DNA encoding genes of interest or nucleic acid sequences of interest for inducing directional integration of two or more different nucleic acids. In some embodiments, two integrases can be used for orthogonal insertion.

The Table 1 below shows examples of pairs of attB site sequence and attP site sequence with different central dinucleotide (CD).

TABLE 1

| Pair | attB | attP | CD |
|---|---|---|---|
| 1 | GGCTTGTCGACGACGGCGTTCTCCG | GTGGTTTGTCTGGTCA | TT |
| | TCGTCAGGATCAT | ACCACCGCGTTCTCA | |
| | (SEQ ID NO: 56) | GTGGTGTACGGTACA | |
| | | AACCCA | |
| | | (SEQ ID NO: 72) | |
| 2 | GGCTTGTCGACGACGGCGAACTCC | GTGGTTTGTCTGGTCA | AA |
| | GTCGTCAGGATCAT | ACCACCGCGAACTCA | |
| | (SEQ ID NO: 57) | GTGGTGTACGGTACA | |
| | | AACCCA | |
| | | (SEQ ID NO: 73) | |
| 3 | GGCTTGTCGACGACGGCGCCCTCC | GTGGTTTGTCTGGTCA | CC |
| | GTCGTCAGGATCAT | ACCACCGCGCCCTCA | |
| | (SEQ ID NO:58) | GTGGTGTACGGTACA | |
| | | AACCCA | |
| | | (SEQ ID NO: 74) | |
| 4 | GGCTTGTCGACGACGGCGGGCTCC | GTGGTTTGTCTGGTCA | GG |
| | GTCGTCAGGATCAT | ACCACCGCGGGCTCA | |
| | (SEQ ID NO:59) | GTGGTGTACGGTACA | |
| | | AACCCA | |
| | | (SEQ ID NO: 75) | |
| 5 | GGCTTGTCGACGACGGCGTGCTCC | GTGGTTTGTCTGGTCA | TG |
| | GTCGTCAGGATCAT | ACCACCGCGTGCTCA | |
| | (SEQ ID NO: 60) | GTGGTGTACGGTACA | |
| | | AACCCA | |
| | | (SEQ ID NO: 76) | |
| 6 | GGCTTGTCGACGACGGCGGTCTCC | GTGGTTTGTCTGGTCA | GT |
| | GTCGTCAGGATCAT | ACCACCGCGGTCTCA | |
| | (SEQ ID NO: 61) | GTGGTGTACGGTACA | |
| | | AACCCA | |
| | | (SEQ ID NO: 38) | |
| 7 | GGCTTGTCGACGACGGCGCTCTCC | GTGGTTTGTCTGGTCA | CT |
| | GTCGTCAGGATCAT | ACCACCGCGCTCTCA | |
| | (SEQ ID NO: 62) | GTGGTGTACGGTACA | |
| | | AACCCA | |
| | | (SEQ ID NO: 77) | |
| 8 | GGCTTGTCGACGACGGCGCACTCC | GTGGTTTGTCTGGTCA | CA |
| | GTCGTCAGGATCAT | ACCACCGCGCACTCA | |
| | (SEQ ID NO: 63) | GTGGTGTACGGTACA | |
| | | AACCCA | |
| | | (SEQ ID NO: 78) | |
| 9 | GGCTTGTCGACGACGGCGTCCTCC | GTGGTTTGTCTGGTCA | TC |
| | GTCGTCAGGATCAT | ACCACCGCGTCCTCA | |
| | (SEQ ID NO: 64) | GTGGTGTACGGTACA | |
| | | AACCCA | |
| | | (SEQ ID NO: 79) | |
| 10 | GGCTTGTCGACGACGGCGGACTCC | GTGGTTTGTCTGGTCA | GA |
| | GTCGTCAGGATCAT | ACCACCGCGGACTCA | |
| | (SEQ ID NO: 65) | GTGGTGTACGGTACA | |
| | | AACCCA | |
| | | (SEQ ID NO: 80) | |
| 11 | GGCTTGTCGACGACGGCGAGCTCC | GTGGTTTGTCTGGTCA | AG |
| | GTCGTCAGGATCAT | ACCACCGCGAGCTCA | |
| | (SEQ ID NO: 66) | GTGGTGTACGGTACA | |
| | | AACCCA | |
| | | (SEQ ID NO: 81) | |
| 12 | GGCTTGTCGACGACGGCGACCTCC | GTGGTTTGTCTGGTCA | AC |
| | GTCGTCAGGATCAT | ACCACCGCGACCTCA | |
| | (SEQ ID NO: 67) | GTGGTGTACGGTACA | |
| | | AACCCA | |
| | | (SEQ ID NO: 82) | |
| 13 | GGCTTGTCGACGACGGCGATCTCC | GTGGTTTGTCTGGTCA | AT |
| | GTCGTCAGGATCAT | ACCACCGCGATCTCA | |
| | (SEQ ID NO: 68) | GTGGTGTACGGTACA | |
| | | AACCCA | |
| | | (SEQ ID NO: 83) | |
| 14 | GGCTTGTCGACGACGGCGGCCTCC | GTGGTTTGTCTGGTCA | GC |
| | GTCGTCAGGATCAT | ACCACCGCGGCCTCA | |
| | (SEQ ID NO: 69) | GTGGTGTACGGTACA | |
| | | AACCCA | |
| | | (SEQ ID NO: 84) | |
| 15 | GGCTTGTCGACGACGGCGCGCTCC | GTGGTTTGTCTGGTCA | CG |
| | GTCGTCAGGATCAT | ACCACCGCGCGCTCA | |
| | (SEQ ID NO: 70) | GTGGTGTACGGTACA | |
| | | AACCCA | |
| | | (SEQ ID NO: 85) | |
| 16 | GGCTTGTCGACGACGGCGTACTCC | GTGGTTTGTCTGGTCA | TA |
| | GTCGTCAGGATCAT | ACCACCGCGTACTCA | |
| | (SEQ ID NO: 71) | GTGGTGTACGGTACA | |
| | | AACCCA | |
| | | (SEQ ID NO: 86) | |

In one aspect, the disclosure provides an integrase or fragment thereof, wherein:
a) the integrase or fragment thereof comprises an amino acid sequence that is at least 90% identical to an amino acid sequence set forth in SEQ ID NO: 1, wherein the integrase binds to the attB nucleic acid set forth in SEQ ID NO: 17 and the attP nucleic acid set forth in SEQ ID NO: 18;
b) the integrase or fragment thereof comprises an amino acid sequence that is at least 90% identical to an amino acid sequence set forth in SEQ ID NO: 2, wherein the integrase binds to the attB nucleic acid set forth in SEQ ID NO: 19 and the attP nucleic acid set forth in SEQ ID NO: 20;
c) the integrase or fragment thereof comprises an amino acid sequence that is at least 90% identical to an amino acid sequence set forth in SEQ ID NO: 3, wherein the integrase binds to the attB nucleic acid set forth in SEQ ID NO: 21 and the attP nucleic acid set forth in SEQ ID NO: 22;
d) the integrase or fragment thereof comprises an amino acid sequence that is at least 90% identical to an amino acid sequence set forth in SEQ ID NO: 4, wherein the integrase binds to the attB nucleic acid set forth in SEQ ID NO: 23 and the attP nucleic acid set forth in SEQ ID NO: 24;
e) the integrase or fragment thereof comprises an amino acid sequence that is at least 90% identical to an amino acid sequence set forth in SEQ ID NO: 5, wherein the integrase binds to the attB nucleic acid set forth in SEQ ID NO: 25 and the attP nucleic acid set forth in SEQ ID NO: 26;
f) the integrase or fragment thereof comprises an amino acid sequence that is at least 90% identical to an amino acid sequence set forth in SEQ ID NO: 6, wherein the integrase binds to the attB nucleic acid set forth in SEQ ID NO: 27 and the attP nucleic acid set forth in SEQ ID NO: 28;
g) the integrase or fragment thereof comprises an amino acid sequence that is at least 90% identical to an amino acid sequence set forth in SEQ ID NO: 7, wherein the integrase binds to the attB nucleic acid set forth in SEQ ID NO: 29 and the attP nucleic acid set forth in SEQ ID NO: 30;
h) the integrase or fragment thereof comprises an amino acid sequence that is at least 90% identical to an amino acid sequence set forth in SEQ ID NO: 8, wherein the integrase binds to the attB nucleic acid set forth in SEQ ID NO: 31 and the attP nucleic acid set forth in SEQ ID NO: 32;
i) the integrase or fragment thereof comprises an amino acid sequence that is at least 90% identical to an amino acid sequence set forth in SEQ ID NO: 9, wherein the integrase binds to the attB nucleic acid set forth in SEQ ID NO: 33 and the attP nucleic acid set forth in SEQ ID NO: 34;
j) the integrase or fragment thereof comprises an amino acid sequence that is at least 90% identical to an amino acid sequence set forth in SEQ ID NO: 10, wherein the integrase binds to the attB nucleic acid set forth in SEQ ID NO: 35 and the attP nucleic acid set forth in SEQ ID NO: 36;
k) the integrase or fragment thereof comprises an amino acid sequence that is at least 90% identical to an amino acid sequence set forth in SEQ ID NO: 11, wherein the integrase binds to the attB nucleic acid set forth in SEQ ID NO: 37 and the attP nucleic acid set forth in SEQ ID NO: 38;
l) the integrase or fragment thereof comprises an amino acid sequence that is at least 90% identical to an amino acid sequence set forth in SEQ ID NO: 12, wherein the integrase binds to the attB nucleic acid set forth in SEQ ID NO: 39 and the attP nucleic acid set forth in SEQ ID NO: 40;
m) the integrase or fragment thereof comprises an amino acid sequence that is at least 90% identical to an amino acid sequence set forth in SEQ ID NO: 13, wherein the integrase binds to the attB nucleic acid set forth in SEQ ID NO: 41 and the attP nucleic acid set forth in SEQ ID NO: 42;
n) the integrase or fragment thereof comprises an amino acid sequence that is at least 90% identical to an amino acid sequence set forth in SEQ ID NO: 14, wherein the integrase binds to the attB nucleic acid set forth in SEQ ID NO: 43 and the attP nucleic acid set forth in SEQ ID NO: 44;
o) the integrase or fragment thereof comprises an amino acid sequence that is at least 90% identical to an amino acid sequence set forth in SEQ ID NO: 15, wherein the integrase binds to the attB nucleic acid set forth in SEQ ID NO: 45 and the attP nucleic acid set forth in SEQ ID NO: 46; or
p) the integrase or fragment thereof comprises an amino acid sequence that is at least 90% identical to an amino acid sequence set forth in SEQ ID NO: 16, wherein the integrase binds to the attB nucleic acid set forth in SEQ ID NO: 47 and the attP nucleic acid set forth in SEQ ID NO: 48.

### Paste

The present disclosure provides non-naturally occurring or engineered systems, methods, and compositions for site-specific genetic engineering using PASTE. PASTE will be discussed in more details below. The PASTE system is described in greater detail in U.S. Provisional Patent Application Ser. No. 63/094,803, filed Oct. 21, 2020, U.S. Provisional Patent Application Ser. No. 63/222,550, filed Jul. 16, 2021, and PCT/US21/56006, filed Oct. 21, 2021, each of which is incorporated herein by reference.

The site-specific genetic engineering disclosed herein is for the insertion of one or more genes of interest or one or more nucleic acid sequences of interest into a genome of a cell. In some embodiments, the gene of interest is a mutated gene implicated in a genetic disease such as, without limitation, a metabolic disease, cystic fibrosis, muscular dystrophy, hemochromatosis, Tay-Sachs, Huntington disease, Congenital Deafness, Sickle cell anemia, Familial hypercholesterolemia, adenosine deaminase (ADA) deficiency, X-linked SCID (X-SCID), and Wiskott-Aldrich syndrome (WAS). In some embodiments, the gene of interest or nucleic acid sequence of interest can be a reporter gene upstream or downstream of a gene for genetic analyses such as, without limitation, for determining the expression of a gene. In some embodiments, the reporter gene is a GFP template or a Gaussia Luciferase (G-Luciferase) template. In some embodiments, the gene of interest or nucleic acid sequence of interest can be used in plant genetics to insert genes to enhance drought tolerance, weather hardiness, and increased yield and herbicide resistance in plants. In some embodiments, the gene of interest or nucleic acid sequence of interest can be used for site-specific insertion of a protein (e.g., a lysosomal enzyme), a blood factor (e.g., Factor I, II, V, VII, X, XI, XII or XIII), a membrane protein, an exon, an intracellular protein (e.g., a cytoplasmic protein, a nuclear protein, an organellar protein such as a mitochondrial protein or lysosomal protein), an extracellular protein, a structural protein, a signaling protein, a regulatory protein, a transport protein, a sensory protein, a motor protein, a defense protein, or a storage protein, an anti-inflammatory signaling molecules into cells for treatment of immune diseases, including but not limited to arthritis, psoriasis, lupus, coeliac disease, glomerulonephritis, hepatitis, and inflammatory bowel disease.

The size of the inserted gene or nucleic acid can vary from about 1 bp to about 50,000 bp. In some embodiments, the size of the inserted gene or nucleic acid can be about 1 bp, 10 bp, 50 bp, 100 bp, 150 bp, 200 bp, 250 bp, 300 bp, 350 bp, 400 bp, 600 bp, 800 bp, 1000 bp, 1200 bp, 1400 bp, 1600 bp, 1800 bp, 2000 bp, 2200 bp, 2400 bp, 2600 bp, 2800 bp, 3000 bp, 3200 bp, 3400 bp, 3600 bp, 3800 bp, 4000 bp, 4200 bp, 4400 bp, 4600 bp, 4800 bp, 5000 bp, 5200 bp, 5400 bp, 5600 bp, 5800 bp, 6000 bp, 6200, 6400 bp, 6600 bp, 6800 bp, 7000 bp, 7200 bp, 7400 bp, 7600 bp, 7800 bp, 8000 bp, 8200 bp, 8400 bp, 8600 bp, 8800 bp, 9000 bp, 9200 bp, 9400 bp, 9600 bp, 9800 bp, 10,000 bp, 10,200 bp, 10,400 bp, 10,600 bp, 10,800 bp, 11,000 bp, 11,200 bp, 11,400 bp, 11,600 bp, 11,800 bp, 12,000 bp, 14,000 bp, 16,000 bp, 18,000 bp, 20,000 bp, 30,000 bp, 40,000 bp, 50,000 bp, or any range that is formed from any two of those values as endpoints.

In some embodiments, the site-specific engineering using the gene of interest or nucleic acid sequence of interest disclosed herein is for the engineering of T cells and NKs for tumor targeting or allogeneic generation. These can involve the use of receptor or CAR for tumor specificity, anti-PD1 antibody, cytokines like IFN-gamma, TNF-alpha, IL-15, IL-12, IL-18, IL-21, and IL-10, and immune escape genes.

In the present disclosure, the site-specific insertion of the gene of interest or nucleic acid of interest is performed through Programmable Addition via Site-Specific Targeting Elements (PASTE). Components for inserting a gene of interest or a nucleic acid of interest using PASTE are for example, without limitation, a nuclease, a gRNA adding the integration site, a DNA or RNA strand comprising the gene or nucleic acid linked to a sequence that is complementary or associated to the integration site, and an integration enzyme. Components for inserting a gene of interest or a nucleic acid of interest using PASTE are for example, without limitation, a prime editor expression, pegRNA adding the integration site, nicking guide RNA, integration enzyme (an integrase, such as an integrase of any one of SEQ ID NOs: 1-16), transgene vector comprising the gene of interest or nucleic acid sequence of interest with gene and integration signal. The nuclease and prime editor integrate the integration site into the genome. The integration enzyme integrates the gene of interest into the integration site. In some embodiments, the transgene vector comprising the gene or nucleic acid sequence of interest with gene and integration signal is a DNA minicircle devoid of bacterial DNA sequences. In some embodiments, the transgenic vector is a eukaryotic or prokaryotic vector.

As used herein, the term “vector” or “transgene vector” refers to a recombinant DNA molecule containing a desired coding sequence and appropriate nucleic acid sequences necessary for the expression of the operably linked coding sequence in a host organism. Nucleic acid sequences necessary for expression in prokaryotes usually include for example, without limitation, a promoter, an operator (optional), a ribosome binding site, and/or other sequences. Eukaryotic cells are generally known to utilize promoters (constitutive, inducible or tissue specific), enhancers, and termination and polyadenylation signals, although some elements may be deleted and other elements added without sacrificing the necessary expression. The transgenic vector may encode the PE and the integration enzyme, linked to each other via a linker. The linker can be a cleavable linker. In some embodiments, the linker can be a non-cleavable linker. In some embodiments the nuclease, prime editor, and/or integration enzyme can be encoded in different vectors.

In one aspect, the disclosure provides a method of inserting multiple genes or nucleic acid sequences of interest into a single site. In some embodiments, multiplexing involves inserting multiple genes of interest in multiple loci using unique pegRNA (Merrick, C. A. et al., ACS Synth. Biol. 2018, 7, 299-310). The insertion of multiple genes of interest or nucleic acids of interest into a cell genome, referred herein as “multiplexing,” is facilitated by incorporation of the complementary 5′ integration site to the 5′ end of the DNA or RNA comprising the first nucleic acid and 3′ integration site to the 3′ end of the DNA or RNA comprising the last nucleic acid. In some embodiments, the number of genome of interest or amino acid sequences of interest that are inserted into a cell genome using multiplexing can be about 1, 2, 3, 4, 5, 6, 7, 8, 9 10, 11, 12, 13, 14, 15, 16, 17, 18, 19, 20, 21, 22, 23, 24, 25, or any range that is formed from any two of those values as endpoints.

In some embodiments, multiplexing allows integration of for example, signaling cascade, over-expression of a protein of interest with its cofactor, insertion of multiple genes mutated in a neoplastic condition, or insertion of multiple CARs for treatment of cancer.

In some embodiments, the integration sites may be inserted into the genome using non-prime editing methods such as rAAV mediated nucleic acid integration, TALENS and ZFNs. A number of unique properties make AAV a promising vector for human gene therapy (Muzyczka, CURRENT TOPICS IN MICROBIOLOGY AND IMMUNOLOGY, 158:97-129 (1992)). Unlike other viral vectors, AAVs have not been shown to be associated with any known human disease and are generally not considered pathogenic. Wild type AAV is capable of integrating into host chromosomes in a site-specific manner M. Kotin et al., PROC. NATL. ACAD. SCI, USA, 87:2211-2215 (1990); R. J. Samulski, EMBO 10(12):3941-3950 (1991)). Instead of creating a double-stranded DNA break, AAV stimulates endogenous homologous recombination to achieve the DNA modification. Further, transcription activator-like effector nucleases (TALENs) and Zinc-finger nucleases (ZFNs) for genome editing and introducing targeted DSBs. The specificity of TALENs arises from two polymorphic amino acids, the so-called repeat variable diresidues (RVDs) located at positions 12 and 13 of a repeated unit. TALENS are linked to FokI nucleases, which cleaves the DNA at the desired locations. ZFNs are artificial restriction enzymes for custom site-specific genome editing. Zinc fingers themselves are transcription factors, where each finger recognizes 3-4 bases. By mixing and matching these finger modules, researchers can customize which sequence to target.

As used herein, the terms “administration,” “introducing,” or “delivery” into a cell, a tissue, or an organ of a plasmid, nucleic acids, or proteins for modification of the host genome refers to the transport for such administration, introduction, or delivery that can occur in vivo, in vitro, or ex vivo. Plasmids, DNA, or RNA for genetic modification can be introduced into cells by transfection, which is typically accomplished by chemical means (e.g., calcium phosphate transfection, polyethyleneimine (PEI) or lipofection), physical means (electroporation or microinjection), infection (this typically means the introduction of an infectious agent such as a virus (e.g., a baculovirus expressing the AAV Rep gene)), transduction (in microbiology, this refers to the stable infection of cells by viruses, or the transfer of genetic material from one microorganism to another by viral factors (e.g., bacteriophages)). Vectors for the expression of a recombinant polypeptide, protein or oligonucleotide may be obtained by physical means (e.g., calcium phosphate transfection, electroporation, microinjection, or lipofection) in a cell, a tissue, an organ or a subject. The vector can be delivered by preparing the vector in a pharmaceutically acceptable carrier for the in vitro, ex vivo, or in vivo delivery to the carrier.

As used herein, the term “transfection” refers to the uptake of an exogenous nucleic acid molecule by a cell. A cell is “transfected” when an exogenous nucleic acid has been introduced into the cell membrane. The transfection can be a single transfection, co-transfection, or multiple transfection. Numerous transfection techniques are generally known in the art. See, for example, Graham et al. (1973) Virology, 52: 456. Such techniques can be used to introduce one or more exogenous nucleic acid molecules into a suitable host cell.

In some embodiments, the exogenous nucleic acid molecule and/or other components for gene editing are combined and delivered in a single transfection. In other embodiments, the exogenous nucleic acid molecule and/or other components for gene editing are not combined and delivered in a single transfection. In some embodiments, exogenous nucleic acid molecule and/or other components for gene editing are combined and delivered in a single transfection to comprise for example, without limitation, a prime editing vector, a landing site such as a landing site containing pegRNA, a nicking guide such as a nicking guide for stimulating prime editing, an expression vector such as an expression vector for a corresponding integrase or recombinase, a minicircle DNA cargo such as a minicircle DNA cargo encoding for green fluorescent protein (GFP), any derivatives thereof, and any combinations thereof. In some embodiments, the gene of interest or amino acid sequence of interest can be introduced using liposomes. In some embodiments, the gene of interest or amino acid sequence of interest can be delivered using suitable vectors for instance, without limitation, plasmids and viral vectors. Examples of viral vectors include, without limitation, adeno-associated viruses (AAV), lentiviruses, adenoviruses, other viral vectors, derivatives thereof, or combinations thereof. The proteins and one or more guide RNAs can be packaged into one or more vectors, e.g., plasmids or viral vectors. In some embodiments, the delivery is via nanoparticles or exosomes. For example, exosomes can be particularly useful in delivery RNA.

In some embodiments, the prime editing inserts the landing site with efficiencies of at least about 1%, at least about 5%, at least about 10%, at least about 15%, at least about, at least about 20%, at least about 25%, at least about 30%, at least about 35%, at least about 40%, at least about 45%, or at least about 50%. In some embodiments, the prime editing inserts the landing site(s) with efficiencies of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, or any range that is formed from any two of those values as endpoints.

### Sequences

Sequences of enzymes, guides, integration sites, and plasmids can be found in the Tables below.

TABLE 2
Integrase enzyme amino acid sequences and the AttB/AttP
nucleic acid sequences recognized by said integrase enzymes.

| Description | Sequence | AttB | AttP |
|---|---|---|---|
| Internal ID: | MEKNRAVLYLRLSKEDVDKVNKGDDS | TGTCTACTAT | TCCATTGCGAG |
| N189929_49_54 | SSIKSQRLLLTDFALERGFKIVGVYSDD | GTCTTTATGC | TGCTAATGATG |
| Name: SsuINT | DESGLYDDRPDFERMMTDAKLDEFDIII | CACATGTGTC | CTTGTGTCGCC |
| Organism/Source: | AKTQSRFSRNMEHIEKYLHHDLPNLGIR | GCATATACAG | ATGGCAGAGCA |
| human gut | FIGAVDGVDTESDENKKSRQINGLVNE | ATAGTAGACA | CATTGC |
| metagenome | WYCEDLSKNIRSAFKAKMKDGQFLGSS | (SEQ ID | (SEQ ID |
| | CPYGYKKDPQNHNHLVVDDYAAKVVQ | NO: 17) | NO: 18) |
| | KIFNLYLEGYGKAKIGSILSSEGILIPTLY | | |
| | KKDILKQNYHNSKALDTTQNWSYQTIH | | |
| | TILNNEVYLGHLIQNKVNTMSYKDKNK | | |
| | RILPKEKWIIVRNTHEPIITEEMFQDVQK | | |
| | LQKNRTRSVENIEPNGLFSGLIFCADCK | | |
| | HAMSRKYARRGEKGFVGYVCKTYKTQ | | |
| | GKNFCESHSIDYDELEEAVLFSIKNEARS | | |
| | ILQQEEIDELRKVQAYDETKSYYEMQLE | | |
| | NIKSRMEKIEKYKKKTYDNYMDDLISR | | |
| | DDYKKYVTEYDKEIGGLKQQQELINSK | | |
| | TDLEKEISTQYDEWVEAFINYVDIDKLT | | |
| | REIVIELIEKIEVNKDGSINIYYKFKNPYI | | |
| | S | | |
| | (SEQ ID NO: 1) | | |
| Internal ID: | MNTVIYARYSAGPRQTDQSIDGQLRVC | GGCCGCGAG | ATGGAGCCGTT |
| N190156_234_12 | TEFCKQRGLTVVDTYCDRHISGRTDERP | GTCGTGTTCG | CTCCGCGGACG |
| Name: SssINT | EFQRLIADAKAHKFEAVVVYKTDRFAR | TCGTCATGTT | TCATGGACTAC |
| Organism/Source: | NKYDSAIYKRELRRNGIQIFYAAEAIPEG | GAGGTTCACG | GGCGTGATCGG |
| human gut | PEGIILESLMEGLAEYYSAELAQKIKRGL | ACCATCACGC | TTTGAA |
| metagenome | NESALKCQSLGSGRPLGYTVDEQKHFQI | C | (SEQ ID |
| | DPESSQAVKTIFEMYIKGESNAAICDYL | (SEQ ID | NO: 20) |
| | NARGLRTSQGNLFNKNSINRIIKNRKYI | NO: 19) | |
| | GEYRYNDIVVEGGMPAIISKETFCMAQ | | |
| | AEMERRRTHRAPVSPKAEYLLAGKLFC | | |
| | GHCKGPMQGVSGTGKSGNKWYYYYC | | |
| | ANTRGKERTCDKKQVSRDRLEKAVVD | | |
| | FTVRYILQENVLEELSKKVYAAQERQN | | |
| | NTASEIAFYEKKLAENKKAIANILRAIES | | |
| | GAMTQALPARLQELENEQTVIQGELSY | | |
| | LKGARLAFTEDQILFALLQHLDPRPGES | | |
| | ERDYHRRIITDFVSEVYLYDDRMLIYFNI | | |
| | SSADGKLKHADLSAIESGVFDAGLISSSS | | |
| | RASSFSTRCALI | | |
| | (SEQ ID NO: 2) | | |
| Internal ID: | MNEKNLEIGAAYIRVSTDDQTELSPDAQ | CATTATATGT | GCTGCCGCTGC |
| N191352_143_72 | LRVILEAAKKDGIIIPQEFVFMEDRGRSG | TTTTACAATC | CTCACCATCTG |
| Name: SscINT | RRADNRPEFQRMISTARQNPSPFRYLYL | CGGGCCGCCA | GGCCGCCATAC |
| Organism/Source: | WKFSRFARNQEESAFYKGILRKKCGVTI | TACTGTAAGA | TGGCTTATAAT |
| human gut | KSVSEPIMEGMFGRLVEMIIEWSDEFYS | ACATATAATG | AAACTG |
| metagenome | VNLSGEVLRGMTQKALEHGYQLTPCLG | (SEQ ID | (SEQ ID |
| | YDAVGHGRPYVINEEQYQIVEFIHRSFF | NO: 21) | NO: 22) |
| | DGKDMTWIAREANRRGYHTRRGNPFD | | |
| | TRAVRIILTNSFYVGLVKWNDVTFQGT | | |
| | HECRESVTSVFSANQERLNRIHRPRGRR | | |
| | QASSCKHWLSGLLKCSICGASLGYNQT | | |
| | KDLTKRGHAFQCWKYTKGIHPGSCSVS | | |
| | SLKAEAAVLESLQMILETGEVEYTYEQR | | |
| | EKHLDDNKLTLIQKSLERLDTKELRIRE | | |
| | AYESGIDTLDEFKTNKARLQRERDQLM | | |
| | EELEELHSQEEPEDVPGKEILIERIQNVY | | |
| | DLLQSPDVDNDDKGNAVRSIIKKIVYIK | | |
| | ESKTFCFYYYV | | |
| | (SEQ ID NO: 3) | | |
| Internal ID: | MERTIKVIQPGTVKIPTKKRVAAYARVS | TATAAACTGA | ATTTGAACCTG |
| N191533_224_76 | SGKDAMLHSLSAQVSYYSNMIQQKNE | TATAATTCAA | TAGTTGGTGCT |
| Name: Ssc2INT | WSYVGIYADEAITGTKDRRVEFNRLIQD | AGTTATAACT | TTATAAATGCG |
| Organism/Source: | CTDGKIDMIITKSISRFARNTLTMLEVVR | TGATATATTC | TAACTAATAAT |
| human gut | KLKNINVDVYFEKENIHSISGDGELMLTI | AAGATGTAG | TCATAT |
| metagenome | LASFAQEESRSVSENCKWRIRKGFEQGE | A | (SEQ ID |
| | LINLRFLYGYRINKGKIEIYEKEAEIVRM | (SEQ ID | NO: 24) |
| | IFDDYLNGEGCTRIGNKLRKMKVNKLR | NO: 23) | |
| | GGMWNSERVVDIIKNEKYTGNALLQK | | |
| | KYVKDHLSKKLVRNKGILTQYYAEGTH | | |
| | PAIIDIKTFEIAQKIMEANRTKFQGKCGS | | |
| | NRYLFTSKIECGICGKNYRHKDREGKST | | |
| | WVCANHLKYGNSRCIAKPLNEEKLKKL | | |
| | INEALELKYFDEEIFIRNIKRIKVTGNQTI | | |
| | EFILKDGKVIEEGMI | | |
| | (SEQ ID NO: 4) | | |
| Internal ID: | MKKIKIDRAIQERPATRKQTRNEKIRQS | AATGAGGTCA | TACAGCGCTAT |
| N203911_45186_ | LTEHVDVQVIPAITDREGYEKPKLRVCA | GACGCATGG | AATAAAGTAGC |
| 6 | YCRVSTDMDTQALSYELQVQNYTDYIR | AGCGCCGCCT | GCCGCGACGCC |
| Name: SsdINT | GNDEWRFAGIYADRGISGTSLKHRDEF | CCGCATGCGT | ATTGCCGCAGA |
| Organism/Source: | NRMIEDCKAGKIDLIITKAVTRFARNVL | CAGGGTCGAT | GCTTGC |
| human gut | DCISTIRMLKQLEHPVAVYFETERINTLD | G | (SEQ ID |
| metagenome | TTSETYLGLISLFAQGESESKSESLKWSY | (SEQ ID | NO: 26) |
| | IRRWKRGTGIYPAWSLLGYEMGEDGK | NO: 25) | |
| | WQIVEAEAELVRIIYDMYLNGYSSPQIA | | |
| | EILTRSGVPTATNQTVWSSGGVLGILRN | | |
| | EKYCGNVLCQKTMTVDVFSHKAIKNTG | | |
| | QKTQYFIEGHHDPIILRSDWDRVQQMID | | |
| | EKYYRKRRGRRTKPRIVLKGCLAGFTQI | | |
| | DLDWDEDDIARIFYSTTPAAEVATPAM | | |
| | ADHIEIIKVKGEN | | |
| | (SEQ ID NO: 5) | | |
| Internal ID: | MKTAAAYIRVSTDDQVEYSPDSQIKLIR | TATTATATCT | AAGCTCATTAT |
| N208621_9_15 | DYAKRNDYILPDEFIFRDDGISGKSAKH | AAAAGCAGT | AAGTCAGTACG |
| Name: SmcINT | RPEFTKMIALAKSPEHPFDAILVWKFSR | ATGGCGGAG | GCGGCCCCGAC |
| Organism/Source: | FARNQEESIVFKNILRKIGVEVRSVSEPIS | CTTAGTGCTT | GGCGAGCTCGG |
| human gut | EDPFGSLVERIIEWTDEYYIINLSGEVKR | TTAGATATAA | CGCTTC |
| metagenome | GMLEKISRGQPVVPPPVGYKMENGQYI | TT | (SEQ ID |
| | PDENAHFIKEIFEAYAAGEGARHIAQRL | (SEQ ID | NO: 28) |
| | AAQGCLTKRGNPIDNRFVDYVLHNPVY | NO: 27) | |
| | IGKLRWSVNSHAASSRHYDSADIIVFDG | | |
| | THEPLISSELWESVQKRLHEVKTLYPKY | | |
| | QRREQPVSFMLKGLVRCSSCGSTLCYC | | |
| | RTSEPSLQCHSYARGSCRQSHSINIATAN | | |
| | EAVIKGLQLAVDKLDFAIAPAKPHYSA | | |
| | DAPGTNKLLAAEYKKMERIKAAYANG | | |
| | TDTLEEYAANKKKISAEIARLEAELQQE | | |
| | SNVKPINKKAFAKRVSEIIKYISDPHNSE | | |
| | AAKNQALRTVISYIIFDRAATTFNIIFHF | | |
| | (SEQ ID NO: 6) | | |
| Internal ID: | MKIAIYARKSKYSPTGESVENQIQLCKE | TGTATCATTT | AACTACGAAGC |
| N675015_95_5 | YLQAKYKSETLEIDEYKDEGYSGGNTN | TCATATAGTG | ATTGCTTGATG |
| Name: UhmINT | RPDFKKLIAQIEDYDMLICYRLDRISRN | TGCAGGTGCT | CAGGTGCTAAT |
| Organism/Source: | VADFSSTLTLLQNNKCDFVSIKEQFDTT | AACTATATGA | TTTGCATCTTCC |
| urban human | SPMGRAMIYISSVFAQLERETIAERIRDN | AAATGATACA | CCCAG |
| microbiome | MMELAKMGRWLGGTIPMGFDSEPITFI | (SEQ ID | (SEQ ID |
| | DENMKERSMTKLIPNVEELKVIELIYEK | NO: 29) | NO: 30) |
| | YLQLGSMGKVVTYLLQNNIKTKKGKDF | | |
| | TLGSIKVILTNPIYVKANQEVVNHLKTQ | | |
| | GITICGDVDGKKALLTYNKTTGISNDVG | | |
| | TKTIVKDKSEWIAAVANHKGIIPADKW | | |
| | LQAQNIKDKNKDSFPALGRSNTTIASRV | | |
| | LRCDKCESTMGVTHGHINPVTGKKHYY | | |
| | YNCTLKKRSKGVRCDNKPAKAAEVDE | | |
| | AILITLENMFKAKSSIIDNLKAKNKARRI | | |
| | EMISSNRVDVINKIIEDKTKQIDNLVNKL | | |
| | SLDDDLTDILFKKIKGLKAEIKELEDELL | | |
| | TLTSDNIKLNEDEVVLDFTEKLLEKCSII | | |
| | RTLDILEQQQIVDALIPLVTWNGDTEVL | | |
| | NIYPLGSPELELKEAESKKK | | |
| | (SEQ ID NO: 7) | | |
| Internal ID: | MKEKVSERKTGAIYIRVSTDKQEELSPD | CGTTATAGGG | GATGCACTGAG |
| N684346_90_69 | AQLRLLLDYAKKDSIDVPKEYIFQDNGI | TATTGCAGTA | CTCACCGTCCG |
| Name : SacINT | SGRKANKRPAFQNMIALAKSKEHPIDTII | CCGACCGCCA | GACCGCCATAC |
| Organism/Source: | VWKFSRFARNQEESIVYKSLLKKNNVD | TACTGTAATA | TGACTTATGAT |
| human gut | VVSVSEPLIDGPFGSLIERIIEWMDEYYSI | CCCTATAACG | ATAAGA |
| metagenome | RLSGEVMRGMTQNAMRGHYQSDAPIG | (SEQ ID | (SEQ ID |
| | YTSPGDKKPPVINPDTVQIPLMIKDMFL | NO: 31) | NO: 32) |
| | SGSTQLQIARKLNDSGYRTKRGNLWDA | | |
| | RGVRYVLENPFYIGKSRWNYTERGRRL | | |
| | KPADEVIYADGNWEALWDEDTFKEIQK | | |
| | RLALNMRKSKSRDISAAKHWLSGLLICS | | |
| | SCGGTLAFGGAHNMRGFQCWKYSKGF | | |
| | CSESHYISTGPIEKMVLEYLEAVMHSPA | | |
| | LSYTVISSSSVDASSKLSDLERQLQKIDA | | |
| | KEKRIKAAYLNEIDTLEEYKANKTALEE | | |
| | ERRTVEKEIEELTLSDVKYSKEDLDKK | | |
| | MKQNISDLLRVLRDESADYIQKGNMMR | | |
| | NVVDHIVFNRKNTSLDVFLKLVV | | |
| | (SEQ ID NO: 8) | | |
| Internal ID: | MKITKKQPLRPRGRSEDKRQSTKNVIRD | GTTTATAAAA | ACGATATTGCC |
| N687611_90_68 | AYINGPQKEVQIIPAKRDMEAETEKKKL | CCGATGCCGC | TGCAAAAGTGC |
| Name: RsaINT | RVCAYCRVSTDEDTQASSYELQVQNYT | TTTGACAGAA | AGACAGAAACG |
| Organism/Source: | RMIRENPEWEFAGIFADEGISGTSVLHR | GCGGAACGG | AGGAACAGAA |
| human gut | EHFLEMIEKCKAGEIDLIITKQVSRFARN | GTTTTAATAA | AAATGGT |
| metagenome | VLDSLNYIFMLRKLDPPVGVYFETEKLN | G | (SEQ ID |
| | TLDKSSDMVITVLSLVAQSESEQKSNSL | (SEQ ID | NO: 34) |
| | KWSFKRRRAQGLGIYPSWALLGYRLDD | NO: 33) | |
| | EKNWEIVEDEADIVRTIYSLYLDGYSST | | |
| | QIAELLTKSGIPTVKGLSVWSSGSVLGIL | | |
| | KNEKFCGDALCQKTVTIDFFTHKSVKN | | |
| | NGIEPQYFVEGHHIPIIEKNDWLLAQQIR | | |
| | KERRYRKRRSTHRKPRIVVKGALSGFMI | | |
| | VDTSWDEEYVDSLLISATQKPEPAPVIA | | |
| | EEDENFIVIEKE | | |
| | (SEQ ID NO: 9) | | |
| Internal ID: | MADIQPVKNGALYIRVSTHLQEELSPDA | GTTAGTACCC | ACAGGGTCTCT |
| N687663_53_29 | QKRLLMEYAEAHNIIVLKEHIYIDSGISG | AAATGATAA | TGCCCGAACTG |
| Name: Rsa2INT | RSARQRPQFNNMIAEAKSKEHPFDVILV | AAGGATGAC | GATGACACAAT |
| Organism/Source: | WKYSRFARNQEESIVYKSMLKRENVDV | CTTTTGTCAT | GGGGATCAAAG |
| human gut | ISVSEPISDDPFGSLIERIIEWMDEYYSIR | TTGGGTACTA | TACTTA |
| metagenome | LSGEVSRGMAENAMRGNYQARPPLGY | AC | (SEQ ID |
| | RIPGYRQTPVIVPEEAELIQLIFDLYTEK | (SEQ ID | NO: 36) |
| | KMGIFEIVRYLNEHGYQTGHKKPFQRR | NO: 35) | |
| | SVTYILKNPTYIGKTIWNQHDQDHKLR | | |
| | DKSEWIIADGKHEPIISKEQFDKAQKRIE | | |
| | STYKPAYRKPTSVCHHWLSSLLKCSSC | | |
| | GRTLVVKRTASKKKDRMYVNFQCYGY | | |
| | QKGICNTNQSISAIKLEPVIMHALEDAM | | |
| | TSGKIHFDVLNPTTLDSSQKQQFLTRLN | | |
| | EIEKKEERIKRAYRDGIDTLEEYKENKSI | | |
| | IQTEKEMLLKKIEHIEEPALSPEEAKPIM | | |
| | MDRIKNVYEIITNPDIGMEEKNKAARSII | | |
| | EKIVFDRATGSVNIFFYLAHCP | | |
| | (SEQ ID NO: 10) | | |
| Accession #: | MRALVVIRLSRVTDATTSPERQLESCQQ | GGCCGGCTTG | GTGGTTTGTCT |
| NP_075302.1 | LCAQRGWDVVGVAEDLDVSGAVDPFD | TCGACGACGG | GGTCAACCACC |
| Name: BxbINT | RKRRPNLARWLAFEEQPFDVIVAYRVD | CGGTCTCCGT | GCGGTCTCAGT |
| Organism/Source: | RLTRSIRHLQQLVHWAEDHKKLVVSAT | CGTCAGGATC | GGTGTACGGTA |
| Mycobacterium | EAHFDTTTPFAAVVIALMGTVAQMELE | ATCCGG | CAAACCCA |
| phage Bxb1 | AIKERNRSAAHFNIRAGKYRGSLPPWG | (SEQ ID | (SEQ ID |
| | YLPTRVDGEWRLVPDPVQRERILEVYH | NO: 37) | NO: 38) |
| | RVVDNHEPLHLVAHDLNRRGVLSPKDY | | |
| | FAQLQGREPQGREWSATALKRSMISEA | | |
| | MLGYATLNGKTVRDDDGAPLVRAEPIL | | |
| | TREQLEALRAELVKTSRAKPAVSTPSLL | | |
| | LRVLFCAVCGEPAYKFAGGGRKHPRYR | | |
| | CRSMGFPKHCGNGTVAMAEWDAFCEE | | |
| | QVLDLLGDAERLEKVWVAGSDSAVEL | | |
| | AEVNAELVDLTSLIGSPAYRAGSPQREA | | |
| | LDARIAALAARQEELEGLEARPSGWEW | | |
| | RETGQRFGDWWREQDTAAKNTWLRS | | |
| | MNVRLTFDVRGGLTRTIDFGDLQEYEQ | | |
| | HLRLGSVVERLHTGMS | | |
| | (SEQ ID NO: 11) | | |
| Accession #: | MTKKVAIYTRVSTTNQAEEGFSIDEQID | CACAATTAAC | GCGAGTTTTTA |
| NP_112664.1 | RLTKYAEAMGWQVSDTYTDAGFSGAK | ATCTCAATCA | TTTCGTTTATTT |
| Name: Tp9INT | LERPAMQRLINDIENKAFDTVLVYKLD | AGGTAAATGC | CAATTAAGGTA |
| (TP901-1) | RLSRSVRDTLYLVKDVFTKNKIDFISLN | T | ACTAAAAAACT |
| Organism/Source: | ESIDTSSAMGSLFLTILSAINEFERENIKE | (SEQ ID | CCTTT |
| Mycobacterium | RMTMGKLGRAKSGKSMMWTKTAFGY | NO: 39) | (SEQ ID |
| phage Bxb1 | YHNRKTGILEIVPLQATIVEQIFTDYLSGI | | NO: 40) |
| | SLTKLRDKLNESGHIGKDIPWSYRTLRQ | | |
| | TLDNPVYCGYIKFKDSLFEGMHKPIIPY | | |
| | ETYLKVQKELEERQQQTYERNNNPRPF | | |
| | QAKYMLSGMARCGYCGAPLKIVLGHK | | |
| | RKDGSRTMKYHCANRFPRKTKGITVYN | | |
| | DNKKCDSGTYDLSNLENTVIDNLIGFQE | | |
| | NNDSLLKIINGNNQPILDTSSFKKQISQID | | |
| | KKIQKNSDLYLNDFITMDELKDRTDSLQ | | |
| | AEKKLLKAKISENKFNDSTDVFELVKTQ | | |
| | LGSIPINELSYDNKKKIVNNLVSKVDVT | | |
| | ADNVDIIFKFQLA | | |
| | (SEQ ID NO: 12) | | |
| Accession #: | MSPFIAPDVPEHLLDTVRVFLYARQSKG | CAGGTTTTTG | TTCGGGTGCTG |
| NP_813744.2 | RSDGSDVSTEAQLAAGRALVASRNAQG | ACGAAAGTG | GGTTGTTGTCT |
| Name: BtlINT | GARWVVAGEFVDVGRSGWDPNVTRA | ATCCAGATGA | CTGGACAGTGA |
| (PhiBT) | DFERMMGEVRAGEGDVVVVNELSRLT | TCCAG | TCCATGGGAAA |
| Organism/Source: | RKGAHDALEIDNELKKHGVRFMSVLEP | (SEQ ID | CTACTCAGCAC |
| Streptomyces | FLDTSTPIGVAIFALIAALAKQDSDLKAE | NO: 41) | CA |
| virus phiBT1 | RLKGAKDEIAALGGVHSSSAPFGMRAV | | (SEQ ID |
| | RKKVDNLVISVLEPDEDNPDHVELVER | | NO: 42) |
| | MAKMSFEGVSDNAIATTFEKEKIPSPGM | | |
| | AERRATEKRLASIKARRLNGAEKPIMW | | |
| | RAQTVRWILNHPAIGGFAFERVKHGKA | | |
| | HINVIRRDPGGKPLTPHTGILSGSKWLEL | | |
| | QEKRSGKNLSDRKPGAEVEPTLLSGWR | | |
| | FLGCRICGGSMGQSQGGRKRNGDLAEG | | |
| | NYMCANPKGHGGLSVKRSELDEFVASK | | |
| | VWARLRTADMEDEHDQAWIAAAAERF | | |
| | ALQHDLAGVADERREQQAHLDNVRRSI | | |
| | KDLQADRKAGLYVGREELETWRSTVL | | |
| | QYRSYEAECTTRLAELDEKMNGSTRVP | | |
| | SEWFSGEDPTAEGGIWASWDVYERREF | | |
| | LSFFLDSVMVDRGRHPETKKYIPLKDRV | | |
| | TLKWAELLKEEDEASEATERELAAL | | |
| | (SEQ ID NO: 13) | | |
| Accession #: | MYPYDVPDYAGSYRPESLDVCIYLRKS | GTAATATGTT | ATAATAGTGTA |
| WP_000286206.1 | RKDVEEERRAIEEGSSYNALERHRKRLF | TGGATATGGG | TATGGTAGAGA |
| Name: BceINT | AIAKAENHNIIDIFEEVASGESIQERPQM | GAAGTGAATC | ATTAAACCAGT |
| Organism/Source: | QQLLRKLEGNEIDGVLVIDLDRLGRGD | AGTACAACCG | TTAATACTCCA |
| Bacillus cereus | MLDAGMIDRAFRYSSTKIITPTDVYDPD | CCACAGTACC | CCATGTACACG |
| AH187 | DESWELVFGIKSLISRQELKSITKRLQNG | CTCATGTCAG | CAGTGAG |
| | RIDSVKEGKHIGKKPPYGYLKDENLRLY | CC | (SEQ ID |
| | PDPEKAWIVKKIFELMCDGKGRQMIAA | (SEQ ID | NO: 44) |
| | ELDRLGIDPPVTKRGAWDSSTITSIIKNE | NO: 43) | |
| | VYTGVIVWGKFKHKKRNGKYTRHKNP | | |
| | QEKWIMYENAHEPIISKELFDAANEAHS | | |
| | SRHKPAVITSKKLTNPLAGILKCKLCGY | | |
| | TMLIQTRKDRPHNYLRCNNPACKGKQK | | |
| | QSVFNLVEEKLLYSLQQIVDEYQAQKV | | |
| | EEVEIDDSKLISFKEKAIISKEKELKELQ | | |
| | AQKGNLHDLLEQGIYTVEIFLERQKNLV | | |
| | ERITSIENDIEVLQKEIETEQIKEHNKTEF | | |
| | IPALKTVIESYHKTTNIELKNQLLKTILST | | |
| | VTYYRHPDWKTNEFEIQVYFKIS | | |
| | (SEQ ID NO: 14) | | |
| Accession #: | MYPYDVPDYAGSAVGIYIRVSTQEQAS | CGCATACATT | CAATAACGGTT |
| WP_012095429.1 | EGHSIESQKKKLASYCEIQGWDDYRFYI | GTTGTTGTTT | GTATTTGTAGA |
| Name: BcyINT | EEGISGKNTNRPKLKLLMEHIEKGKINIL | TTCCAGATCC | ACTTGACCAGT |
| Organism/Source: | LVYRLDRLTRSVIDLHKLLNFLQEHGCA | AGTTGGTCCT | TGTTTTAGTAA |
| Bacillus | FKSATETYDTTTANGRMSMGIVSLLAQ | GTAAATATAA | CATAAATACAA |
| cytotoxicus | WETENMSERIKLNLEHKVLVEGERVGA | GCAATCCATG | CTCCGAATA |
| NVH 391-98 | IPYGFDLSDDEKLVKNEKSAILLDMVER | TGAGT | (SEQ ID |
| | VENGWSVNRIVNYLNLTNNDRNWSPN | (SEQ ID | NO: 46) |
| | GVLRLLRNPALYGATRWNDKIAENTHE | NO: 45) | |
| | GIISKERFNRLQQILADRSIHHRRDVKGT | | |
| | YIFQGVLRCPVCDQTLSVNRFIKKRKDG | | |
| | TEYCGVLYRCQPCIKQNKYNLAIGEARF | | |
| | LKALNEYMSTVEFQTVEDEVIPKKSERE | | |
| | MLESQLQQIARKREKYQKAWASDLMS | | |
| | DDEFEKLMVETRETYDECKQKLESCED | | |
| | PIKIDETYLKEIVYMFHQTFNDLESEKQ | | |
| | KEFISKFIRTIRYTVKEQQPIRPDKSKTG | | |
| | KGKQKVIITEVEFYQS | | |
| | (SEQ ID NO: 15) | | |
| Accession #: | MYPYDVPDYAGSKVAIYTRVSSAEQAN | GTTCGTGGTA | TTTTTGTATGTT |
| WP_014533238.1 | EGYSIHEQKKKLISYCEIHDWNEYKVFT | ACTATGGGTG | AGTIGTGTCAC |
| Name: SluINT | DAGISGGSMKRPALQKLMKHLSSFDLV | GTACAGGTGC | TGGGTAGACCT |
| Organism/Source: | LVYKLDRLTRNVRDLLDMLEEFEQYNV | CACATTAGTT | AAATAGTGACA |
| Staphylococcus | SFKSATEVFDTTSAIGKLFITMVGAMAE | GTACCATTTA | CAACTGCTATT |
| lugdunensis | WERETIRERSLFGSRAAVREGNYIREAP | TGTTTATGTG | AAAATTTAA |
| N920143 | FCYDNIEGKLHPNEYAKVIDLIVSMFKK | GTTAAC | (SEQ ID |
| | GISANEIARRLNSSKVHVPNKKSWNRNS | (SEQ ID | NO: 48) |
| | LIRLMRSPVLRGHTKYGDMLIENTHEPV | NO: 47) | |
| | LSEHDYNAINNAISSKTHKSKVKHHAIF | | |
| | RGALVCPQCNRRLHLYAGTVKDRKGY | | |
| | KYDVRRYKCETCSKNKDVKNVSFNESE | | |
| | VENKFVNLLKSYELNKFHIRKVEPVKKI | | |
| | EYDIDKINKQKINYTRSWSLGYIEDDEY | | |
| | FELMEEINATKKMIEEQTTENKQSVSKE | | |
| | QIQSINNFILKGWEELTIKDKEELILSTV | | |
| | DKIEFNFIPKDKKHKTNTLDINNIHFKFS | | |
| | (SEQ ID NO: 16) | | |

TABLE 3
Integrase enzyme nucleic acid sequences

| Description | Sequence |
|---|---|
| Name: | ATGAATGAGAAGAACCTTGAGATAGGGGCT |
| SscINT | GCATACATTCGGGTCAGCACCGACGACCAG |
| | ACTGAACTGTCTCCCGATGCTCAGCTGCGG |
| | GTAATCCTTGAGGCGGCCAAGAAAGACGGG |
| | ATTATAATTCCTCAGGAGTTCGTGTTCATG |
| | GAGGACAGAGGCCGTTCCGGCCGCCGGGCT |
| | GATAACAGACCTGAGTTCCAGAGGATGATT |
| | TCCACCGCTCGACAGAATCCTTCTCCATTC |
| | AGGTATTTATACCTTTGGAAGTTCAGTCGG |
| | TTCGCAAGAAATCAGGAGGAATCAGCTTTC |
| | TACAAGGGAATTCTGCGGAAAAAGTGCGGC |
| | GTGACGATCAAATCTGTTAGTGAGCCCATT |
| | ATGGAGGGCATGTTCGGGCGCTTGGTAGAA |
| | ATGATCATCGAATGGTCTGATGAATTCTAC |
| | AGCGTTAACCTCAGCGGTGAAGTCCTCAGG |
| | GGAATGACGCAAAAGGCATTAGAGCATGGA |
| | TACCAGTTAACCCCCTGCCTGGGCTACGAT |
| | GCTGTGGGACATGGAAGACCGTACGTCATC |
| | AACGAGGAGCAGTATCAGATTGTTGAATTT |
| | ATCCACCGCAGCTTTTTCGATGGTAAGGAT |
| | ATGACGTGGATTGCTAGGGAAGCTAACAGA |
| | AGGGGATATCACACTCGCAGGGGGAATCCA |
| | TTCGATACCAGGGCAGTGAGAATCATCCTG |
| | ACCAATTCTTTCTATGTGGGACTCGTGAAA |
| | TGGAACGACGTAACATTTCAAGGCACACAT |
| | GAGTGCCGGGAAAGCGTGACTTCTGTATTC |
| | TCCGCGAATCAGGAAAGGCTGAATCGTATT |
| | CACCGACCAAGGGGGCGGCGACAGGCCTCT |
| | TCCTGTAAACACTGGCTGAGCGGCCTCCTG |
| | AAGTGCTCAATATGCGGAGCTAGTCTGGGC |
| | TACAACCAGACCAAAGACCTGACAAAGCGA |
| | GGTCATGCTTTCCAGTGCTGGAAGTACACC |
| | AAAGGAATTCATCCTGGCTCTTGCAGCGTA |
| | TCCTCTCTCAAAGCAGAGGCGGCCGTTCTG |
| | GAGTCCCTGCAAATGATATTGGAAACTGGA |
| | GAGGTCGAGTATACCTACGAACAGCGCGAG |
| | AAGCACCTGGATGATAACAAACTCACCCTC |
| | ATCCAGAAGTCCTTGGAACGACTTGACACC |
| | AAAGAGCTGCGAATTCGAGAGGCTTACGAG |
| | TCTGGAATAGATACCTTGGATGAGTTCAAG |
| | ACAAATAAGGCACGACTGCAGCGAGAGCGT |
| | GATCAACTCATGGAAGAGCTTGAAGAATTG |
| | CACTCTCAAGAGGAGCCAGAGGATGTCCCC |
| | GGCAAGGAGATCTTAATCGAACGTATTCAA |
| | AATGTATACGATTTGCTGCAATCCCCAGAT |
| | GTCGATAATGATGATAAAGGCAACGCCGTG |
| | CGGTCAATTATCAAGAAGATAGTGTATATT |
| | AAGGAATCTAAAACTTTCTGTTTTTATTAT |
| | TATGTG |
| | (SEQ ID NO: 49) |
| Name: | ATGAAGGAGAAGGTGAGTGAGAGAAAAACA |
| SacINT | GGCGCCATTTACATAAGAGTTTCTACGGAC |
| | AAACAGGAAGAGCTTTCACCAGACGCACAG |
| | CTGAGGCTCCTCCTGGACTACGCTAAGAAA |
| | GATTCTATCGATGTTCCTAAGGAGTACATC |
| | TTCCAAGATAACGGCATTAGTGGGCGAAAA |
| | GCGAACAAGCGCCCCGCGTTCCAGAATATG |
| | ATCGCACTCGCGAAGTCCAAAGAGCACCCA |
| | ATCGACACAATCATTGTGTGGAAGTTCTCT |
| | CGCTTTGCCCGGAATCAGGAGGAATCAATT |
| | GTGTACAAGAGTTTACTCAAAAAAAACAAC |
| | GTCGATGTGGTGAGTGTGTCCGAGCCTCTG |
| | ATTGATGGGCCATTTGGAAGCCTGATTGAG |
| | AGAATTATTGAGTGGATGGACGAGTATTAT |
| | TCCATTCGATTGTCTGGCGAGGTGATGCGT |
| | GGTATGACTCAAAATGCCATGCGGGGGCAT |
| | TACCAGAGCGATGCACCGATTGGGTACACA |
| | TCCCCAGGGGACAAAAAGCCCCCGGTTATA |
| | AACCCGGATACCGTTCAGATTCCTCTGATG |
| | ATCAAAGATATGTTCTTAAGCGGCTCAACC |
| | CAGCTGCAAATTGCCAGAAAGCTCAACGAC |
| | AGTGGCTATAGGACAAAGCGCGGTAACCTG |
| | TGGGACGCGAGAGGCGTCCGGTACGTCCTG |
| | GAGAACCCGTTTTACATCGGGAAAAGCCGC |
| | TGGAATTACACGGAGAGAGGGCGACGGCTG |
| | AAGCCGGCAGATGAGGTGATATACGCTGAC |
| | GGGAACTGGGAGGCACTGTGGGATGAGGAC |
| | ACCTTCAAGGAGATCCAAAAAAGATTGGCA |
| | CTGAATATGCGCAAGTCCAAGTCTAGGGAC |
| | ATCTCAGCTGCAAAACACTGGCTGAGCGGT |
| | CTCTTAATCTGTTCTTCCTGCGGCGGAACC |
| | CTGGCCTTCGGGGGAGCACACAATATGAGG |
| | GGGTTTCAATGCTGGAAATACTCAAAGGGG |
| | TTCTGCAGCGAATCCCATTATATCAGCACC |
| | GGTCCAATTGAGAAAATGGTTCTGGAGTAC |
| | TTAGAGGCCGTCATGCACTCCCCTGCGCTG |
| | AGTTACACGGTTATCAGTAGTTCATCCGTC |
| | GATGCCAGCTCCAAACTGTCAGACCTGGAG |
| | CGCCAATTGCAGAAAATAGACGCCAAGGAG |
| | AAACGCATCAAGGCAGCATACCTCAACGAA |
| | ATAGATACACTGGAGGAGTACAAAGCTAAT |
| | AAAACAGCCTTGGAGGAAGAACGCCGTACC |
| | GTCGAGAAGGAAATCGAGGAGCTCACCCTC |
| | AGCGACGTGAAATATTCTAAGGAGGACCTT |
| | GACAAGAAAATGAAGCAGAATATATCAGAC |
| | CTGCTGCGGGTGCTGAGAGACGAATCTGCC |
| | GATTACATCCAGAAAGGTAACATGATGAGA |
| | AACGTGGTCGATCATATCGTCTTTAACAGG |
| | AAGAATACTAGCCTGGACGTTTTTCTGAAA |
| | TTAGTAGTG |
| | (SEQ ID NO: 50) |
| Name: | TACCCTTATGACGTACCTGATTACGCCGGT |
| BceINT | AGCTACAGGCCAGAATCCCTCGACGTATGC |
| | ATTTACCTTCGCAAATCCAGGAAGGACGTT |
| | GAAGAAGAACGCCGCGCAATCGAAGAAGGC |
| | AGCTCCTACAACGCACTGGAACGGCATCGG |
| | AAGCGATTGTTTGCCATTGCCAAGGCAGAA |
| | AATCACAACATCATCGATATTTTTGAAGAA |
| | GTTGCCAGTGGAGAGAGCATACAGGAAAGA |
| | CCCCAAATGCAGCAGCTGCTCAGGAAGTTG |
| | GAAGGCAATGAAATTGATGGCGTGCTGGTG |
| | ATTGATCTCGATAGACTCGGGCGGGGCGAT |
| | ATGCTGGATGCGGGAATGATCGATCGTGCA |
| | TTCAGATACTCATCTACCAAAATTATCACC |
| | CCAACAGATGTCTACGATCCTGATGACGAA |
| | AGTTGGGAGCTGGTGTTCGGGATTAAGAGT |
| | TTAATCAGCCGACAGGAGCTCAAGTCCATC |
| | ACCAAACGACTGCAGAATGGCCGGATCGAT |
| | TCAGTGAAGGAGGGGAAGCACATTGGCAAG |
| | AAGCCACCTTATGGCTACTTGAAGGATGAG |
| | AATCTGAGGCTGTATCCAGATCCAGAAAAG |
| | GCCTGGATTGTGAAGAAGATTTTTGAACTG |
| | ATGTGTGACGGAAAGGGACGGCAGATGATT |
| | GCGGCTGAGTTGGACAGACTGGGTATTGAC |
| | CCCCCTGTGACGAAAAGGGGAGCATGGGAC |
| | TCTAGTACCATCACCAGTATTATAAAGAAC |
| | GAAGTTTATACAGGCGTCATTGTCTGGGGG |
| | AAATTTAAGCATAAAAAGAGGAATGGTAAG |
| | TATACGCGGCATAAGAACCCACAGGAGAAG |
| | TGGATTATGTACGAGAACGCCCATGAACCC |
| | ATTATATCCAAAGAGCTTTTCGATGCGGCA |
| | AACGAAGCCCATAGCTCCAGACACAAGCCC |
| | GCTGTCATAACGAGTAAAAAGCTGACTAAC |
| | CCACTGGCTGGCATCTTGAAGTGCAAGTTG |
| | TGTGGCTACACAATGCTCATACAGACTCGG |
| | AAGGACAGGCCTCATAACTACTTACGATGT |
| | AACAATCCAGCCTGTAAGGGCAAGCAAAAA |
| | CAGTCAGTTTTCAATTTAGTGGAGGAGAAG |
| | TTGCTCTATTCACTGCAGCAAATCGTGGAC |
| | GAGTACCAGGCCCAGAAAGTTGAAGAGGTC |
| | GAAATTGATGATTCTAAACTCATCTCTTTT |
| | AAGGAAAAGGCAATAATCTCCAAAGAGAAG |
| | GAGCTTAAGGAGTTACAAGCTCAGAAAGGC |
| | AACCTGCATGACCTGCTCGAACAAGGTATT |
| | TACACGGTCGAAATCTTCCTGGAACGGCAG |
| | AAGAATTTGGTGGAAAGAATAACCAGCATC |
| | GAGAACGACATCGAGGTGCTGCAGAAGGAG |
| | ATTGAAACTGAGCAGATCAAAGAACACAAT |
| | AAGACCGAGTTCATCCCCGCCTTAAAAACG |
| | GTGATCGAATCATATCACAAAACAACCAAT |
| | ATTGAACTCAAAAACCAGCTGCTGAAGACC |
| | ATTCTGAGCACCGTGACATACTATAGGCAT |
| | CCCGACTGGAAAACCAATGAATTTGAAATC |
| | CAGGTGTACTTCAAAATCtcct |
| | (SEQ ID NO: 51) |

TABLE 4
Linker Sequences

| Descrip- | | Amino acid |
|---|---|---|
| tion | Sequence (5′-3′) | sequence |
| A-P2A | GGAAGCGGAGCTACTAACTTCAGCCT | GSGATNFSLLK |
| | GCTGAAGCAGGCTGGCGACGTGGAGG | QAGDVEENPGP |
| | AGAACCCTGGACCT | (SEQ ID |
| | (SEQ ID NO: 87) | NO: 96) |
| B- | GGGGGAGGAGGTTCTGGAGGCGGAGG | GGGGSGGGGS |
| (GGGS)3 | CTCCGGAGGCGGAGGGTCA | GGGGS |
| (G-3x) | (SEQ ID NO: 88) | (SEQ ID |
| | | NO: 97) |
| C-GGGGS | GGAGGTGGCGGGAGC | GGGGS |
| | (SEQ ID NO: 89) | (SEQ ID |
| | | NO: 98) |
| D-PAPAP | CCCGCACCAGCGCCT | PAPAP |
| | (SEQ ID NO: 90) | (SEQ ID |
| | | NO: 99) |
| E- | GAGGCAGCTGCCAAGGAAGCCGCT | EAAAKEAAAKE |
| (EAAAK)3 | GCCAAGGAGGCGGCCGCAAAG | AAAK |
| | (SEQ ID NO: 91) | (SEQ ID |
| | | NO: 100) |
| F-XTEN | AGTGGGAGCGAGACCCCTGGGACT | SGSETPGTSES |
| | AGCGAGTCAGCTACACCCGAAAGC | ATPES |
| | (SEQ ID NO: 92) | (SEQ ID |
| | | NO: 101) |
| G-(GGS)6 | GGGGGGTCAGGTGGATCCGGCGG | GGSGGSGGSGG |
| | AAGTGGCGGATCCGGTGGATCTGG | SGGSGGS |
| | CGGCAGT | (SEQ ID |
| | (SEQ ID NO: 93) | NO: 102) |
| H-EAAAK | GAAGCTGCTGCTAAG | EAAAK |
| | (SEQ ID NO: 94) | (SEQ ID |
| | | NO: 103) |
| MCP-MLV | GCTGGCAGCGAGACACCAGGAAC | AGSETPGTSES |
| Linker | AAGCGAGTCAGCAACACCAGAGA | ATPESSGGSSG |
| | GCAGTGGCGGCAGCAGCGGCGGC | GSST |
| | AGCAGCACC | (SEQ ID |
| | (SEQ ID NO: 95) | NO: 104) |

TABLE 5
Exemplary Cas9 nuclease and Reverse
Transcriptase

| Descrip- | |
|---|---|
| tion | Sequence |
| SpCas9 | DKKYSIGLDIGTNSVGWAVITDEYKVPSKKFKV |
| Amino | LGNTDRHSIKKNLIGALLFDSGETAEATRLKRT |
| acid | ARRRYTRRKNRICYLQEIFSNEMAKVDDSFFHR |
| SEQ ID | LEESFLVEEDKKHERHPIFGNIVDEVAYHEKYP |
| NO: 52 | TIYHLRKKLVDSTDKADLRLIYLALAHMIKFRG |
| | HFLIEGDLNPDNSDVDKLFIQLVQTYNQLFEEN |
| | PINASGVDAKAILSARLSKSRRLENLIAQLPGE |
| | KKNGLFGNLIALSLGLTPNFKSNFDLAEDAKLQ |
| | LSKDTYDDDLDNLLAQIGDQYADLFLAAKNLSD |
| | AILLSDILRVNTEITKAPLSASMIKRYDEHHQD |
| | LTLLKALVRQQLPEKYKEIFFDQSKNGYAGYID |
| | GGASQEEFYKFIKPILEKMDGTEELLVKLNRED |
| | LLRKQRTFDNGSIPHQIHLGELHAILRRQEDFY |
| | PFLKDNREKIEKILTFRIPYYVGPLARGNSRFA |
| | WMTRKSEETITPWNFEEVVDKGASAQSFIERMT |
| | NFDKNLPNEKVLPKHSLLYEYFTVYNELTKVKY |
| | VTEGMRKPAFLSGEQKKAIVDLLFKTNRKVTVK |
| | QLKEDYFKKIECFDSVEISGVEDRFNASLGTYH |
| | DLLKIIKDKDFLDNEENEDILEDIVLTLTLFED |
| | REMIEERLKTYAHLFDDKVMKQLKRRRYTGWGR |
| | LSRKLINGIRDKQSGKTILDFLKSDGFANRNFM |
| | QLIHDDSLTFKEDIQKAQVSGQGDSLHEHIANL |
| | AGSPAIKKGILQTVKVVDELVKVMGRHKPENIV |
| | IEMARENQTTQKGQKNSRERMKRIEEGIKELGS |
| | QILKEHPVENTQLQNEKLYLYYLQNGRDMYVDQ |
| | ELDINRLSDYDVDAIVPQSFLKDDSIDNKVLTR |
| | SDKNRGKSDNVPSEEVVKKMKNYWRQLLNAKLI |
| | TQRKFDNLTKAERGGLSELDKAGFIKRQLVETR |
| | QITKHVAQILDSRMNTKYDENDKLIREVKVITL |
| | KSKLVSDFRKDFQFYKVREINNYHHAHDAYLNA |
| | VVGTALIKKYPKLESEFVYGDYKVYDVRKMIAK |
| | SEQEIGKATAKYFFYSNIMNFFKTEITLANGEI |
| | RKRPLIETNGETGEIVWDKGRDFATVRKVLSMP |
| | QVNIVKKTEVQTGGFSKESILPKRNSDKLIARK |
| | KDWDPKKYGGFDSPTVAYSVLVVAKVEKGKSKK |
| | LKSVKELLGITIMERSSFEKNPIDFLEAKGYKE |
| | VKKDLIIKLPKYSLFELENGRKRMLASAGELQK |
| | GNELALPSKYVNFLYLASHYEKLKGSPEDNEQK |
| | QLFVEQHKHYLDEIIEQISEFSKRVILADANLD |
| | KVLSAYNKHRDKPIREQAENIIHLFTLTNLGAP |
| | AAFKYFDTTIDRKRYTSTKEVLDATLIHQSITG |
| | LYETRIDLSQLGGD |
| RT(1-478)- | LNIEDEYRLHETSKEPDVSLGSTWLSDFPQAWA |
| Sto7d | ETGGMGLAVRQAPLIIPLKATSTPVSIKQYPMS |
| Amino | QEARLGIKPHIQRLLDQGILVPCQSPWNTPLLP |
| acid | VKKPGTNDYRPVQDLREVNKRVEDIHPTVPNPY |
| SEQ ID | NLLSGPPPSHQWYTVLDLKDAFFCLRLHPTSQP |
| NO: 53 | LFAFEWRDPEMGISGQLTWTRLPQGFKNSPTLF |
| | NEALHRDLADFRIQHPDLILLQYVDDLLLAATS |
| | ELDCQQGTRALLQTLGNLGYRASAKKAQICQKQ |
| | VKYLGYLLKEGQRWLTEARKETVMGQPTPKTPR |
| | QLREFLGKAGFCRLFIPGFAEMAAPLYPLTKPG |
| | TLFNWGPDQQKAYQEIKQALLTAPALGLPDLTK |
| | PFELFVDEKQGYAKGVLTQKLGPWRRPVAYLSK |
| | KLDPVAAGWPPCLRMVAAIAVLTKDAGKLTMGQ |
| | PLVILAPHAVEALVKQPPDRWLSNARMTHYQAL |
| | LLDTDRVQFGPVVALNPATLLPLPEEGLQHNCL |
| | DGTGGGGVTVKFKYKGEELEVDISKIKKVWRVG |
| | KMISFTYDDNGKTGRGAVSEKDAPKELLQMLEK |
| | SGKKSGGSKRTADGS |

TABLE 7
Exemplary Nucleic Acid Binding Proteins and
Protein-Recruiting Stem-Loop Nucleic Acid
Sequences

| Descrip- | |
|---|---|
| tion | Sequence |
| MS2 Coat | MASNFTQFVLVDNGGTGDVTVAPSNFANGVAEWIS |
| Protein | SNSRSQAYKVTCSVRQSSAQKRKYTIKVEVPKVAT |
| (MCP) | QTVGGVELPVAAWRSYLNMELTIPIFATNSDCELI |
| Amino | VKAMQGLLKDGNPIPSAIAANSGIYSA |
| Acid | (SEQ ID NO: 105) |
| MS2 Coat | ATGGCTTCAAACTTTACTCAGTTCGTGCTCGTGGA |
| Protein | CAATGGTGGGACAGGGGATGTGACAGTGGCTCCTT |
| (MCP) | CTAATTTCGCTAATGGGGTGGCAGAGTGGATCAGC |
| Nucleic | TCCAACTCACGGAGCCAGGCCTACAAGGTGACATG |
| Acid | CAGCGTCAGGCAGTCTAGTGCCCAGAAGAGAAAGT |
| | ATACCATCAAGGTGGAGGTCCCCAAAGTGGCTACC |
| | CAGACAGTGGGCGGAGTCGAACTGCCTGTCGCCGC |
| | TTGGAGGTCCTACCTGAACATGGAGCTCACTATCC |
| | CAATTTTCGCTACCAATTCTGACTGTGAACTCATC |
| | GTGAAGGCAATGCAGGGGCTCCTCAAAGACGGTAA |
| | TCCTATCCCTTCCGCCATCGCCGCTAACTCAGGTA |
| | TCTACAGCGCT |
| | (SEQ ID NO: 106) |
| MS2 | ACAUGAGGAUCACCCAUGU |
| Stem- | (SEQ ID NO:54) |
| Loop | |

### EXAMPLES

While several experimental Examples are contemplated, these Examples are intended to be non-limiting.

### Example 1

### Bxb1 Integration Data Lenti Reporter

The PASTE system, including the description in Example 1 and Example 2, are described in greater detail in U.S. Provisional Patent Application Ser. No. 63/094,803, filed Oct. 21, 2020, and U.S. Provisional Patent Application Ser. No. 63/222,550, filed Jul. 16, 2021, each of which is incorporated herein by reference.

Serine integrase Bxb1 has been shown to be more active than Cre recombinase and highly efficient in bacteria and mammalian cells for irreversible integration of target genes. FIG. 1 and FIG. 2 show schematics of PASTE methodology using Bxb1 (Merrick, C. A. et al., ACS Synth. Biol. 2018, 7, 299-310).

To probe the efficiency of the Bxb1 integration system, a clonal HEK293FT cell line with attB Bxb1 site

(SEQ ID NO: 37)
(GGCCGGCTTGTCGACGACGGCGGTCTCCGTCGTCAGGATCATCCGG)

integrated using lentivirus was developed. The modified HEK293FT cell line was then transferred with the following plasmids: (1) plus/minus Bxb1 expression plasmid and (2) plus/minus GFP or G-Luc minicircle template with attP Bxb1 site. After 72 hours, the integration of GFP or Gluc into the attB site in the HEK293FT genome was probed. The percent integrations of GFP or Gluc into the attB locus are shown in FIG. 3. It was observed that GFP and Gluc showed efficient integration into the attB site in HEK293FT cells.

### Example 2

### Addition of Bxb1 Site to Human Genome Using PRIME

The maximum length of attB that can be integrated into a HEK293FT cell line with the best efficiency was probed. To probe the best length of attB

(SEQ ID NO: 37)
(GGCCGGCTTGTCGACGACGGCGGTCTCCGTCGTCAGGATCATCCGG)

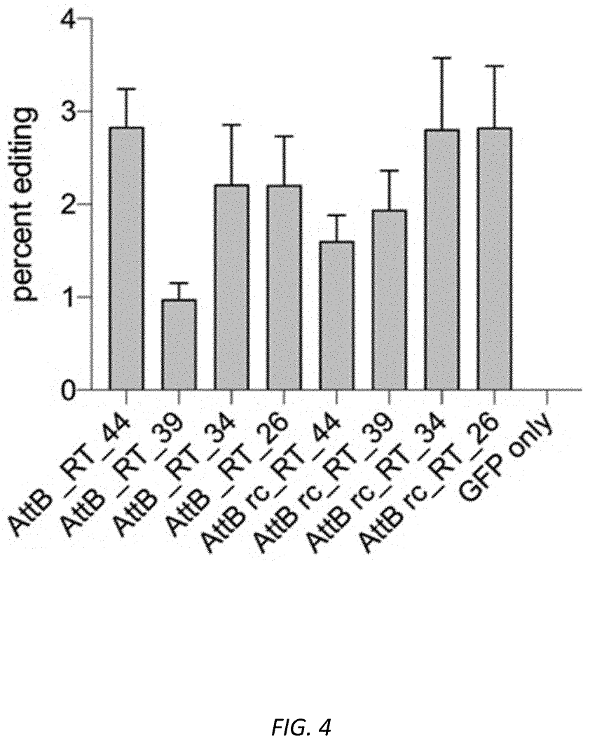

or its reverse complement attP (CCGGATGATCCTGACGACGGAGACCGCCGTCGTCGACAAGCCGGCC)(SEQ ID NO:107) for prime editing, pegRNAs having PBS length of 13 nt with varying RT homology length were used. The following plasmids were transfected in HEK293FT: (1) prime expression plasmid; (2) HEK3 targeting pegRNA design; and (3) HEK3+90 nicking guide. After 72 hours, the percent integration of each of the attB construct was probed. FIG. 4 shows the percent editing in each HEK3 targeting pegRNA. It was observed that attB with 44, 34 and 26 base pairs and attB reverse complement with 34 and 26 base pairs showed the highest percent editing.

### Example 3

### Integrase Discovery Platform & Use in PASTE System

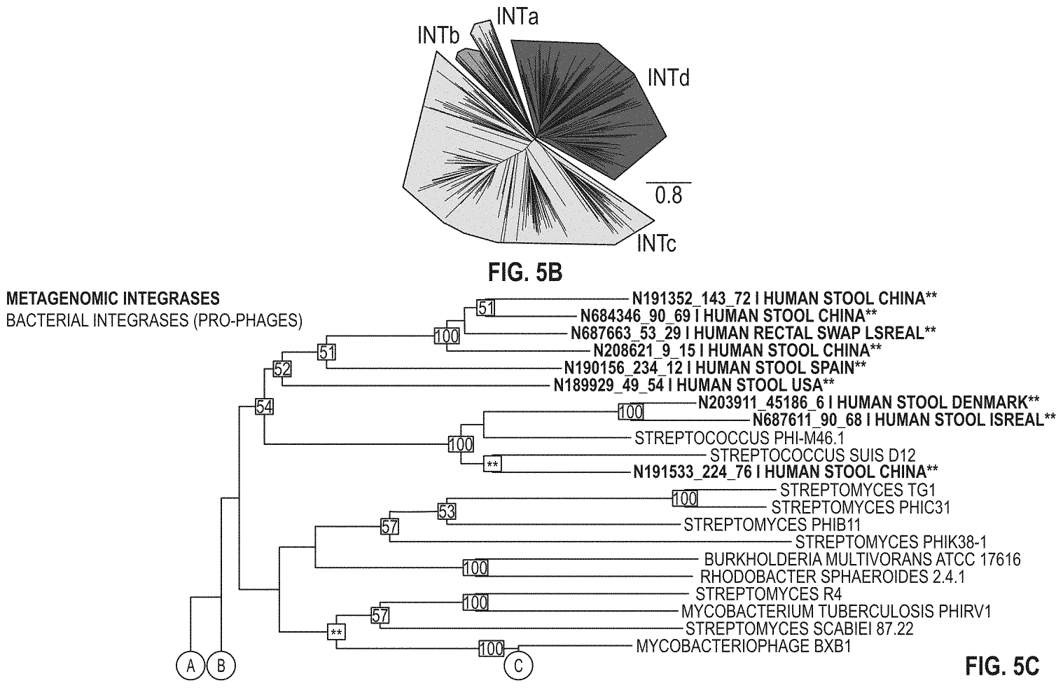

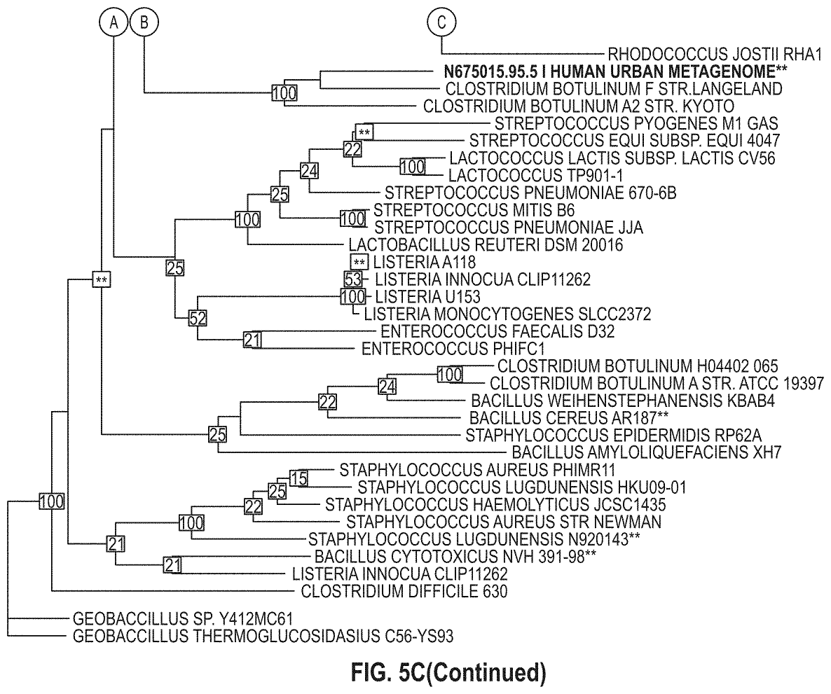

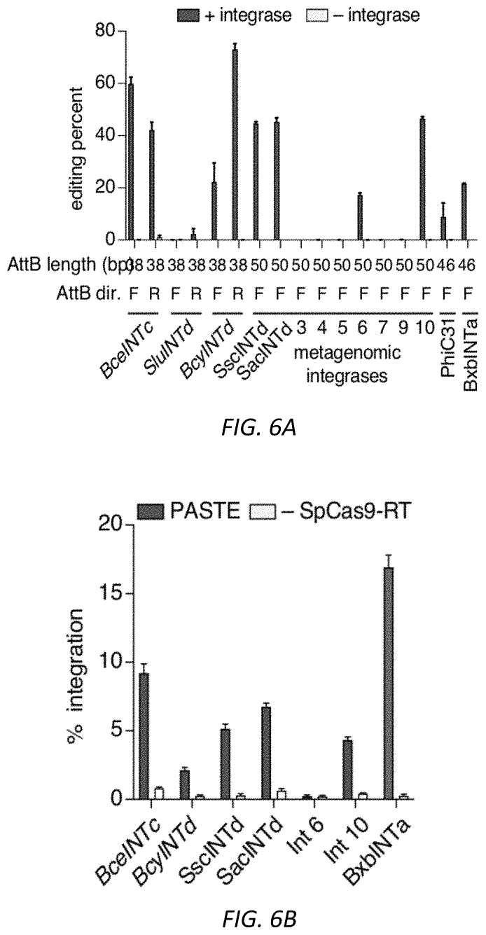

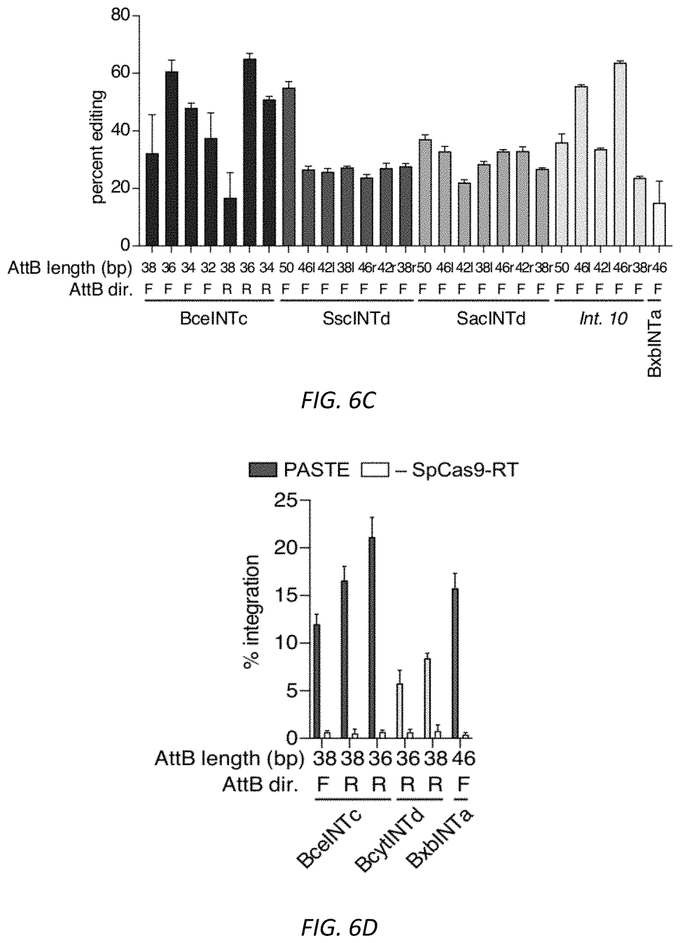

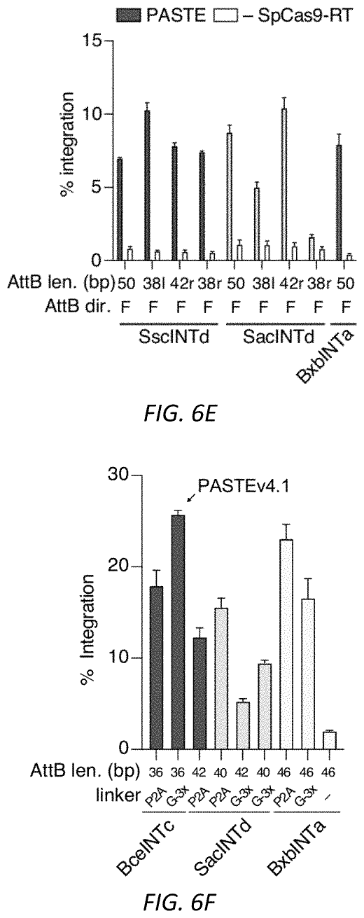

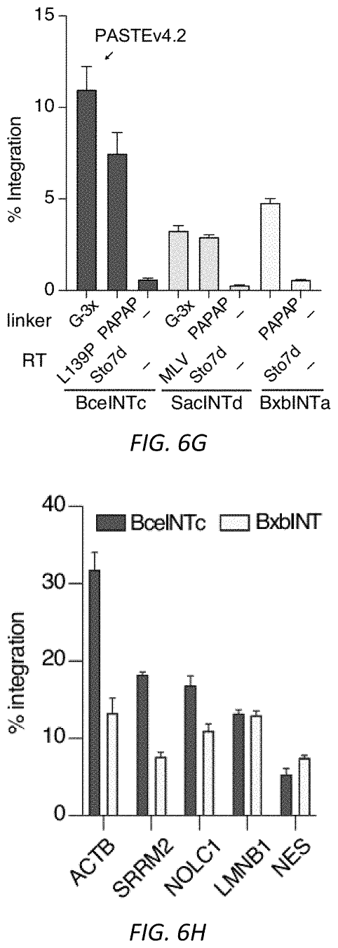

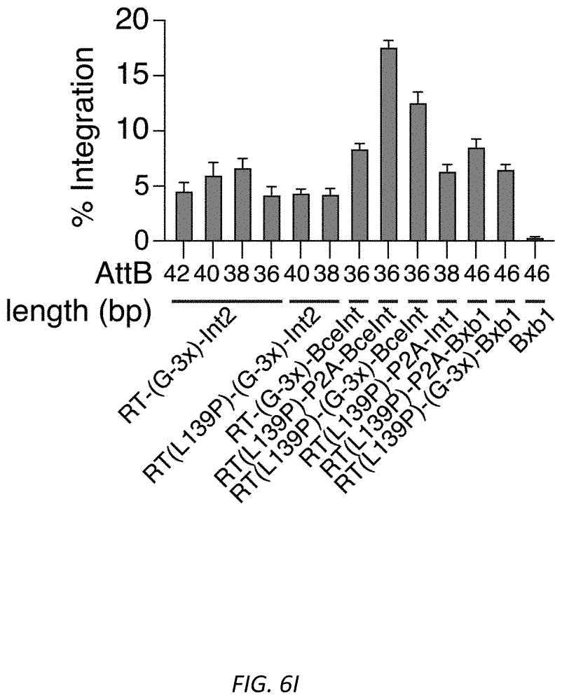

Integrase choice can have implications for integration activity. To identify novel integrases with improved activity in the PASTE system, bacterial and metagenomic sequences were mined for new phage associated serine integrases (FIG. 5A). Exploring over 10 TB worth of data from NCBI, JGI, and other sources, 27,399 novel integrases were found (FIG. 5B, FIG. 5C) and their associated attachment sites were annotated using a novel repeat finding algorithm that could predict potential 50 bp attachment sites with high confidence near phage boundaries. Analysis of the integrases sequences revealed that they fell into four distinct clusters: INTa, INTb, INTc, and INTd. About half of integrases (14,771) derive from metagenomic sequences, presumably from pro-phages, and 13,693 of the integrases specifically derive from human microbiome metagenomic samples. An initial screen of integrase activity using a reporter system revealed that a number of the integrases were highly active in HEK293FT cells with more activity than BxbINT, a member of the INTa family (FIG. 6A). Using the predicted 50 bp sequences encoded in attachment site-containing guide RNAs (atgRNAs) along with minicircles containing the complementary AttP sites, it was found that these integrases were compatible with PASTE but with lower efficiency than BxbINTa-based PASTE (FIG. 6B). It was hypothesized that this was because of their longer 50 bp AttB sequences and so truncations of these AttBs were explored in the hopes of finding more minimal attachment sites. Truncation screening on integrase reporters revealed that AttB truncations of all the integrases, including as short as 34 bp, were still active and many had more activity than BxbINTa (FIG. 6C). Upon porting these new shorter AttBs to atgRNAs for PASTE, it was found that a number of integrases had more activity in the PASTE system than BxbINT-based PASTE at the ACTB locus, including the integrase from B. cereues (BceINTc), N191352_143_72 stool sample from China (SscINTd), and N684346_90_69 stool sample from adult in China (SacINTd), while others like the integrase from B. cytotoxicus (BcytlNTd) and S. lugdunensis (SluINTd) did not (FIG. 6A and FIG. 6D-FIG. 6E). Because of its superior efficiency when used with PASTE, BceINTc when used as PASTE is referred to as PASTEv4.1. Moreover, upon optimization of these integrases with different linkers and RT domains, it was found that BceINTc fused to SpCas9-RTSto7d or SpCas9-MLV-RT^{L139P} variant had the most activity, even higher than BxbINTa-based PASTE (FIG. 6G-FIG. 6I). The construct SpCas9-MLV-RT^{L139P}-BceINTc construct is referred to as PASTEv4.1. We then evaluated this optimized PASTEv4.1 and found that across a number of endogenous gene loci that it performed better than BxbINTa-based PASTE (FIG. 6H and FIG. 6J).

### Example 4

### RNA-Based Reverse Transcriptase Recruitment

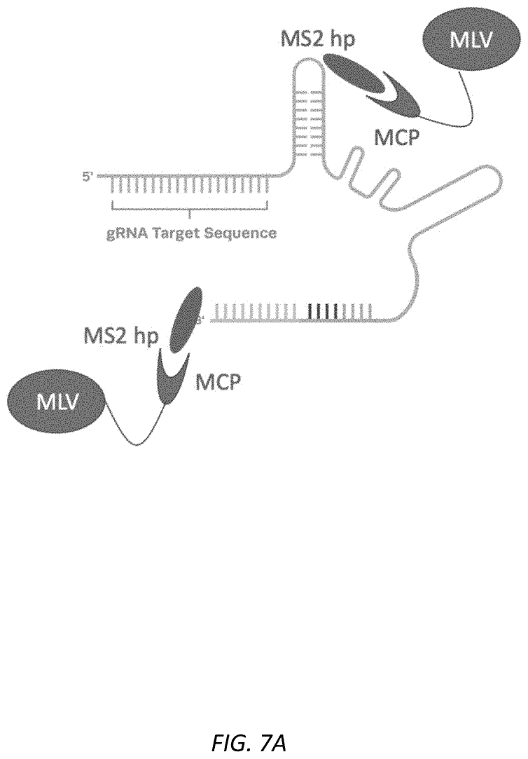

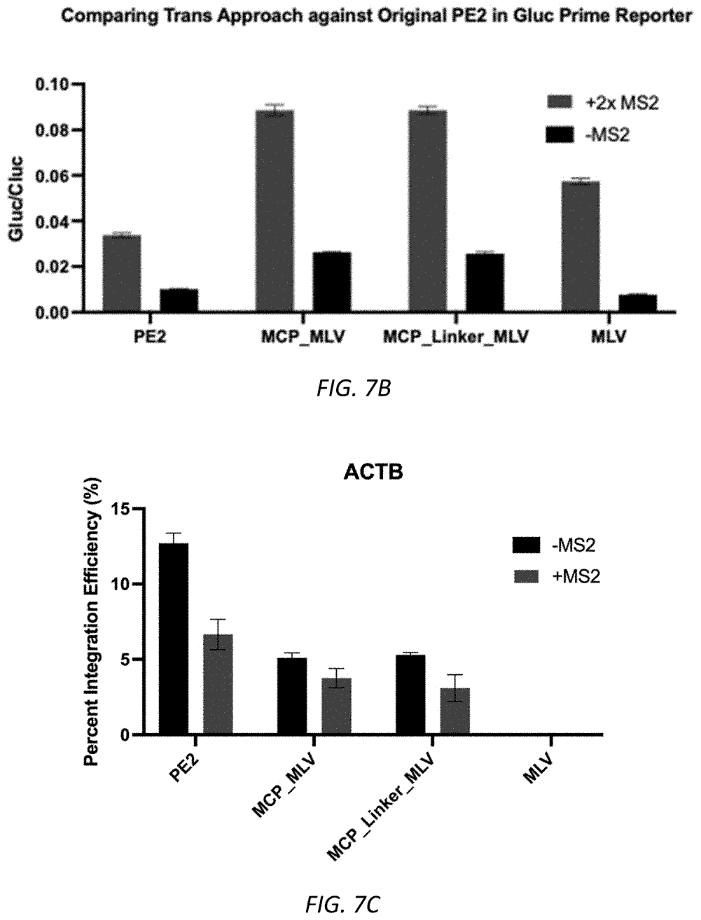

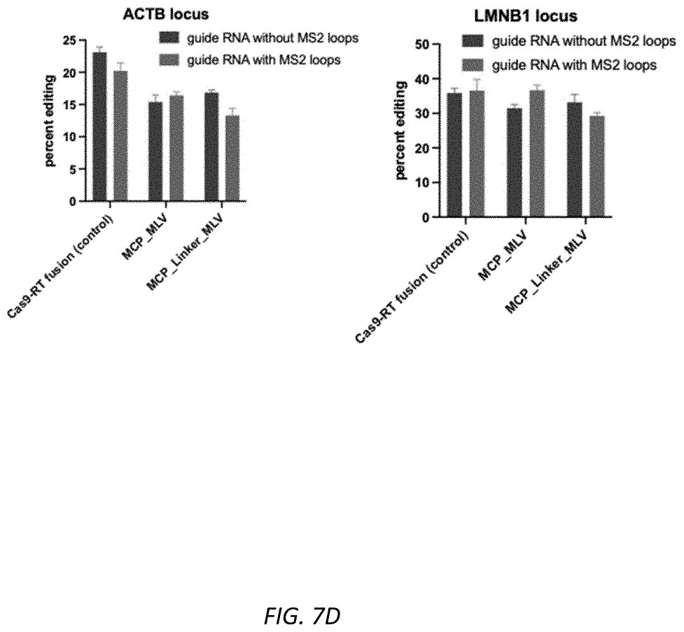

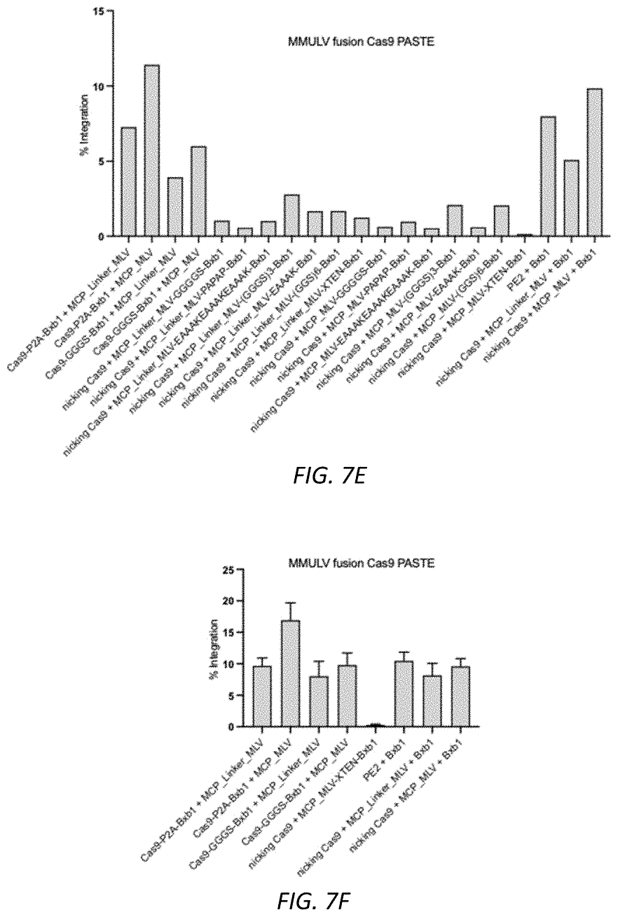

In addition to the fusions of nucleases and reverse transcriptases in PASTE systems, reverse transcriptases can be recruited in trans to a pegRNA in via RNA-based interaction. MS2 hairpins encoded in the pegRNA sequence allow for recruitment of MS2-coat protein (MCP) fused to Murine Leukemia Virus (MLV) reverse transcriptase as shown in the diagram in FIG. 7A. Comparing the effect of fused or physically separate nucleases and reverse transcriptases reveals robust editing efficiency with the Gluc prime editing assay when reverse transcriptase is recruited to the RNA in trans (FIG. 7B). RNA-based recruitment of reverse transcriptase has variable effects at different endogenous loci, with the ACTB loci showing decreased editing with the trans approach and the LMNB1 locus showing similar editing efficiency between the two approaches (FIG. 7C-FIG. 7D). Further, integration efficiency of the PASTE system could be dramatically influenced by combining different iterations of PASTE with RNA-based recruitment of reverse transcriptases (FIG. 7E and FIG. 7F).

One skilled in the art will appreciate further features and advantages of the disclosure based on the above-described embodiments. Accordingly, the disclosure is not to be limited by what has been particularly shown and described, except as indicated by the appended claims.

1-86. (canceled)

87. A complex for genome editing comprising:
(i) an RNA-guided nuclease;
(ii) a fusion protein comprising a reverse transcriptase domain linked to a nucleic acid binding protein; and
(iii) at least one guide RNA (gRNA) comprising a 5′ end and a 3′ end and comprising at least one protein-recruiting stem-loop nucleic acid sequence,
wherein the protein-recruiting stem-loop nucleic acid sequence binds to the nucleic acid binding protein.

88. The complex of claim 87, wherein the nucleic acid binding protein is MS2 coat protein (MCP) PP7 coat protein, or streptavidin.

89. The complex of claim 87, wherein the protein-recruiting stem-loop nucleic acid sequence is a MS2 sequence, PP7 stem loop sequence, or S1 aptamer sequence.

90. The complex of claim 88, wherein the MS2 sequence comprises a nucleic acid sequence of ACAUGAGGAUCACCCAUGU (SEQ ID NO:54) or sequence of >90% similarity.

91. The complex of claim 87, wherein the gRNA comprises a primer binding site (PBS), a reverse transcriptase (RT) template sequence, and an integration site sequence.

92. (canceled)

93. (canceled)

94. (canceled)

95. The complex of claim 87, wherein the protein-recruiting stem-loop nucleic acid sequence is present at the 5′ end of the gRNA, the 3′ end of the gRNA, or both.

96. (canceled)

97. (canceled)

98. (canceled)

99. The complex of claim 87, wherein the RNA-guided nuclease comprises a CRISPR nuclease.

100. The complex of claim 99, wherein the CRISPR nuclease is Cas9 or Cas12.

101. The complex of claim 99, wherein the CRISPR nuclease comprises nickase activity.

102. The complex of claim 99, wherein the CRISPR nuclease is selected from Cas9-D10A, Cas9-H840A, and Cas12a/b nickase.

103. The complex of claim 87, wherein the reverse transcriptase domain is selected from the group consisting of Moloney Murine Leukemia Virus (M-MLV) reverse transcriptase domain, transcription xenopolymerase (RTX), avian myeloblastosis virus reverse transcriptase (AMV-RT), and Eubacterium rectale maturase RT (MarathonRT).

104. The complex of claim 87, wherein the reverse transcriptase domain comprises a mutation relative to the wild-type sequence or contains a stabilization domain optionally wherein the stabilization domain comprises a DNA-binding Sto7d protein from Sulfolobus tokodaii.

105. The complex of claim 103, wherein the M-MLV reverse transcriptase domain comprises one or more mutations selected from the group consisting of D200N, T306K, W313F, T330P, L603W, and L139P.

106. The complex of claim 87, wherein the reverse transcriptase domain is linked to the nucleic acid binding protein via a cleavable or noncleavable linker.

107. (canceled)

108. (canceled)

109. The complex of claim 106, comprising any one or more of the linker sequences recited in Table 4.

110. The complex of claim 87, wherein one or both of the RNA-guided nuclease and fusion protein are linked to an integration enzyme or fragment thereof.

111. The complex of claim 110, wherein the integration enzyme is selected from the group consisting of Cre, Dre, Vika, Bxb1, BceINT φC31, RDF, FLP, φBT1, R1, R2, R3, R4, R5, TP901-1, A118, φFC1, φC1, MR11, TG1, φ370.1, Wβ, BL3, SPBc, K38, Peaches, Veracruz, Rebeuca, Theia, Benedict, KSSJEB, PattyP, Doom, Scowl, Lockley, Switzer, Bob3, Troube, Abrogate, Anglerfish, Sarfire, SkiPole, ConceptII, Museum, Severus, Airmid, Benedict, Hinder, ICleared, Sheen, Mundrea, BxZ2, φRV, retrotransposases encoded by R2, L1, Tol2 Tc1, Tc3, Mariner Himar 1, Mariner mos 1, and Minos, and any mutants thereof.

112. (canceled)

113. (canceled)

114. The complex of claim 110, wherein the integration enzyme comprises an amino acid sequence that is at least 90% identical to an amino acid sequence set forth in any one of SEQ ID NOs: 1-16.

115. The complex of claim 110, wherein the integration enzyme recognizes an integration site.

116. The complex of claim 115, wherein the integration site is an attB site, an attP site, an attL site, an attR site, a lox71 site a Vox site, or a FRT site.

117. The complex of claim 115, wherein the integration enzyme recognizes nucleic acid attachment sites attB and attP, other recognition site pairs, or any pseudosites in a human genome.

118. The complex of claim 116, wherein the attB and/or attP nucleic acid sequence is between 12 and 60 nucleotides in length or between 18 and 50 nucleotides in length.

119. The complex of claim 116, wherein the attB and/or attP nucleic acid sequence comprises one or more truncations.

120. (canceled)

120. The complex of claim 116, wherein the integration enzyme binds to any one of the attB nucleic acid sequences selected from the group consisting of SEQ ID NOs: 17, 19, 21, 23, 25, 27, 29, 31, 33, 35, 37, 39, 41, 43, 45, and 47.

121. The complex of claim 116, wherein the integration enzyme binds to any one of the attP nucleic acid sequences selected from the group consisting of SEQ ID NOs: 18, 20, 22, 24, 26, 28, 30, 32, 34, 36, 38, 40, 42, 44, 46, and 48.

122. The complex of claim 110, wherein:
a) the integrase or fragment thereof comprises an amino acid sequence that is at least 90% identical to an amino acid sequence set forth in SEQ ID NO: 1, wherein the integrase binds to the attB nucleic acid set forth in SEQ ID NO: 17 and the attP nucleic acid set forth in SEQ ID NO: 18;
b) the integrase or fragment thereof comprises an amino acid sequence that is at least 90% identical to an amino acid sequence set forth in SEQ ID NO: 2, wherein the integrase binds to the attB nucleic acid set forth in SEQ ID NO: 19 and the attP nucleic acid set forth in SEQ ID NO: 20;
c) the integrase or fragment thereof comprises an amino acid sequence that is at least 90% identical to an amino acid sequence set forth in SEQ ID NO: 3, wherein the integrase binds to the attB nucleic acid set forth in SEQ ID NO: 21 and the attP nucleic acid set forth in SEQ ID NO: 22;
d) the integrase or fragment thereof comprises an amino acid sequence that is at least 90% identical to an amino acid sequence set forth in SEQ ID NO: 4, wherein the integrase binds to the attB nucleic acid set forth in SEQ ID NO: 23 and the attP nucleic acid set forth in SEQ ID NO: 24;
e) the integrase or fragment thereof comprises an amino acid sequence that is at least 90% identical to an amino acid sequence set forth in SEQ ID NO: 5, wherein the integrase binds to the attB nucleic acid set forth in SEQ ID NO: 25 and the attP nucleic acid set forth in SEQ ID NO: 26;
f) the integrase or fragment thereof comprises an amino acid sequence that is at least 90% identical to an amino acid sequence set forth in SEQ ID NO: 6, wherein the integrase binds to the attB nucleic acid set forth in SEQ ID NO: 27 and the attP nucleic acid set forth in SEQ ID NO: 28;
g) the integrase or fragment thereof comprises an amino acid sequence that is at least 90% identical to an amino acid sequence set forth in SEQ ID NO: 7, wherein the integrase binds to the attB nucleic acid set forth in SEQ ID NO: 29 and the attP nucleic acid set forth in SEQ ID NO: 30;
h) the integrase or fragment thereof comprises an amino acid sequence that is at least 90% identical to an amino acid sequence set forth in SEQ ID NO: 8, wherein the integrase binds to the attB nucleic acid set forth in SEQ ID NO: 31 and the attP nucleic acid set forth in SEQ ID NO: 32;
i) the integrase or fragment thereof comprises an amino acid sequence that is at least 90% identical to an amino acid sequence set forth in SEQ ID NO: 9, wherein the integrase binds to the attB nucleic acid set forth in SEQ ID NO: 33 and the attP nucleic acid set forth in SEQ ID NO: 34;
j) the integrase or fragment thereof comprises an amino acid sequence that is at least 90% identical to an amino acid sequence set forth in SEQ ID NO: 10, wherein the integrase binds to the attB nucleic acid set forth in SEQ ID NO: 35 and the attP nucleic acid set forth in SEQ ID NO: 36;
k) the integrase or fragment thereof comprises an amino acid sequence that is at least 90% identical to an amino acid sequence set forth in SEQ ID NO: 11, wherein the integrase binds to the attB nucleic acid set forth in SEQ ID NO: 37 and the attP nucleic acid set forth in SEQ ID NO: 38;
l) the integrase or fragment thereof comprises an amino acid sequence that is at least 90% identical to an amino acid sequence set forth in SEQ ID NO: 12, wherein the integrase binds to the attB nucleic acid set forth in SEQ ID NO: 39 and the attP nucleic acid set forth in SEQ ID NO: 40;
m) the integrase or fragment thereof comprises an amino acid sequence that is at least 90% identical to an amino acid sequence set forth in SEQ ID NO: 13, wherein the integrase binds to the attB nucleic acid set forth in SEQ ID NO: 41 and the attP nucleic acid set forth in SEQ ID NO: 42;
n) the integrase or fragment thereof comprises an amino acid sequence that is at least 90% identical to an amino acid sequence set forth in SEQ ID NO: 14, wherein the integrase binds to the attB nucleic acid set forth in SEQ ID NO: 43 and the attP nucleic acid set forth in SEQ ID NO: 44;
o) the integrase or fragment thereof comprises an amino acid sequence that is at least 90% identical to an amino acid sequence set forth in SEQ ID NO: 15, wherein the integrase binds to the attB nucleic acid set forth in SEQ ID NO: 45 and the attP nucleic acid set forth in SEQ ID NO: 46; or
p) the integrase or fragment thereof comprises an amino acid sequence that is at least 90% identical to an amino acid sequence set forth in SEQ ID NO: 16, wherein the integrase binds to the attB nucleic acid set forth in SEQ ID NO: 47 and the attP nucleic acid set forth in SEQ ID NO: 48.

123. (canceled)

124. (canceled)

125. (canceled)

126. (canceled)

127. (canceled)

128. (canceled)

129. (canceled)

130. (canceled)

131. (canceled)

132. (canceled)

133. (canceled)

134. A method of site-specific integration of a nucleic acid into a cell genome, the method comprising:
(a) incorporating an integration site at a desired location in the cell genome by introducing into the cell:
i. an RNA-guided nuclease comprising a nickase activity;
ii. a fusion protein comprising a reverse transcriptase domain linked to a nucleic acid binding protein; and
iii. a guide RNA (gRNA) comprising a 5′ end and a 3′ end and comprising a primer binding sequence linked to an integration sequence and at least one protein-recruiting stem-loop nucleic acid sequence, wherein the protein-recruiting stem-loop nucleic acid sequence binds to the nucleic acid binding protein, wherein the gRNA interacts with the RNA-guided nuclease and targets the desired location in the cell genome, wherein the RNA-guided nuclease nicks a strand of the cell genome and the reverse transcriptase domain incorporates the integration sequence of the gRNA into the nicked site, thereby providing the integration site at the desired location of the cell genome; and
(b) integrating the nucleic acid into the cell genome by introducing into the cell:
i. a DNA or RNA strand comprising the nucleic acid linked to a sequence that is complementary or associated to the integration site; and
ii. an integration enzyme or fragment thereof, wherein the integration enzyme or fragment thereof incorporates the nucleic acid into the cell genome at the integration site by integration, recombination, or reverse transcription of the sequence that is complementary or associated to the integration site, thereby introducing the nucleic acid into the desired location of the cell genome of the cell.

135. (canceled)

136. (canceled)

137. (canceled)

138. (canceled)

139. (canceled)

140. (canceled)

141. (canceled)

142. (canceled)

144. (canceled)

145. (canceled)

146. (canceled)

147. (canceled)

148. (canceled)

149. (canceled)

150. (canceled)

151. (canceled)

152. (canceled)

151. (canceled)

152. (canceled)

153. (canceled)

154. (canceled)

155. (canceled)

156. (canceled)

157. (canceled)

158. (canceled)

159. (canceled)

160. (canceled)

161. (canceled)

162. (canceled)

162. (canceled)

163. (canceled)

164. (canceled)

165. (canceled)

166. (canceled)

167. (canceled)

168. (canceled)

169. (canceled)
